SEQUENCE LISTING (129 sequences)

SEQ ID NO: 1 - protein, 178 aa, Homo sapiens
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 178 aa, Artificial Sequence
  Lipocalin mutein var.13
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 3 - protein, 178 aa, Artificial Sequence
  Lipocalin mutein var.12
QDSTSDLIPAPPLSKVPLQQNFQDNQFHGKWYVVGQAGNIKLREDKDPNKMMATIYELKEDKSYNVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 4 - protein, 178 aa, Artificial Sequence
  Lipocalin mutein var.14
QDSTSDLIPAPPLSKVPLQQNFQDNQFHGKWYVVGQAGNIRLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 5 - protein, 178 aa, Artificial Sequence
  Lipocalin mutein var.15
QDSTSDLIPAPPLSKVPLQQNFQDNQFHGKWYVVGQAGNIKLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 6 - protein, 175 aa, Artificial Sequence
  Lipocalin mutein var.16
QDSTSDLIPAPPLSKVPLQQNFQDNQFHGKWYVVGQAGNIKLREDSKMMATIYELKEDKSYDVTGVSFDDKKCTYAIMTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 7 - protein, 178 aa, Artificial Sequence
  Lipocalin mutein var.17
QDSTSDLIPAPPLSKVPLQQNFQDNQFHGKWYVVGQAGNIKLREDKDPVKMMATIYELKEDKSYDVTGVTFDDKKCRYDISTFVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 8 - protein, 178 aa, Artificial Sequence
  Lipocalin mutein var.18
QDSTSDLIPAPPLSKVPLQQNFQDNQFHGKWYVVGQAGNIRLREDKDPHKMMATIYELKEDKSYDVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 9 - protein, 178 aa, Artificial Sequence
  Lipocalin mutein var.19
QDSTSDLIPAPPLSKVPLQQNFQDNQFHGKWYVVGQAGNIKLREDKDPNKMMATIYELKEDKSYDVTGVTFDDKKCTYAISTLVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 10 - protein, 178 aa, Artificial Sequence
  Lipocalin mutein var.20
QDSTSDLIPAPPLSKVPLQQNFQDNQFHGKWYVVGQAGNIRLREDKDPSKMMATIYELKEDKSYDVTAVTFDDKKCNYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 11 - protein, 178 aa, Artificial Sequence
  Lipocalin mutein var.47
QDSTSDLIPAPPLSKVPLQQNFQDNQFHGKWYVVGMAGNNLLREDKDPHKMSATIYELKEDKSYNVTDVMFLDKKCQYIIWTFVPGSQPGEFTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 12 - protein, 178 aa, Artificial Sequence
  Lipocalin mutein var.48
QDSTSDLIPAPPLSKVPLQQNFQDNQFQGKWYVVGMAGNNLLREDKDPHKMSATIYELKEDKSYNVTDVMFLDKKCQYIIWTFVPGSQPGELTLGFIRSDLGHTSYLVRVVSTNYNQHAMVFFKSVIQNREWFGITLYGRTKELTSELKENFIRFSKSLGLPENHIVFPVPIDQCIDG

SEQ ID NO: 13 - protein, 178 aa, Artificial Sequence
  Lipocalin mutein var.49
QDSTSDLIPAPPLSKVPLQQNFQDYQFQGKWYVVGMAGNNLLREDKDPHKMGATIYELKEDKSYNVTDVMLLDKKCQYIIQTFVPGSQPGESTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 14 - protein, 178 aa, Artificial Sequence
  Lipocalin mutein var.50
QDSTSDLIPAPPLSKVPLQQNFQDNQFQGKWYVVGMAGNNLLREDKDPHKMGATIYELKEDKSYNVTDVMFLDKKCQHIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 15 - protein, 178 aa, Artificial Sequence
  Lipocalin mutein var.51
QDSTSDLIPAPPLSKVPLQQNFQDDQFQGKWYVVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 16 - protein, 178 aa, Artificial Sequence
  Lipocalin mutein var.52
QDSTSDLIPAPPLSKVPLQQNFQDNQFQGKWYIVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 17 - protein, 178 aa, Artificial Sequence
  Lipocalin mutein var.53
QDSTSDLIPAPPLSKVPLQRNFQDNQFQGKWYVVGMAGNNLLRVDKDPHKMGATIYELKEDKSYNVTDVMFLDKKCQYIIWTFVPGSQPGELTLGFIKSDPGHTSYLVRVVSTNYNQHAMVYFKSVIQNREWFGITLYGRTKELTSELKENFIRFSKSLGLPENHIVFPVPIDQCIDG

SEQ ID NO: 18 - protein, 178 aa, Artificial Sequence
  Lipocalin mutein var.54
QDSTSDLIPAPPLSKVPLQQNFQDNQFQGKWYVVGMAGNNLLREDKDPHKMSATIYELKEDKSYNVTDVMFLDKKCQYINWPFVPGSQPGEFTLGFIKSDLGPTSYLVRVVSTNYNQHAMVFFKSVIQNREWFGITLYGRTKELTSELKENFIRFSKSLGLPENHIVFPVPIDQCIDG

SEQ ID NO: 19 - protein, 178 aa, Artificial Sequence
  Lipocalin mutein var.55
QDSTSDLIPAPPLSKVPLQQNFQDNQFQGKWYVVGMAGNNLLREDKDPHKMGATIYELNEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 20 - protein, 178 aa, Artificial Sequence
  Lipocalin mutein var.56
QDSTSDLIPAPPLSKVPLQQNFQDNQFQGKWYVVGMAGNNLLRDDKDPHKMSATIYELKEDKSYNVTDVMLLDKKCHYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 21 - protein, 5 aa, Artificial Sequence
  FAP(4B9)  CDR-H1
SYAMS

SEQ ID NO: 22 - protein, 17 aa, Artificial Sequence
  FAP(4B9)  CDR-H2
AIIGSGASTYYADSVKG

SEQ ID NO: 23 - protein, 8 aa, Artificial Sequence
  FAP(4B9)  CDR-H3
GWFGGFNY

SEQ ID NO: 24 - protein, 12 aa, Artificial Sequence
  FAP(4B9)  CDR-L1
RASQSVTSSYLA

SEQ ID NO: 25 - protein, 7 aa, Artificial Sequence
  FAP(4B9)  CDR-L2
VGSRRAT

SEQ ID NO: 26 - protein, 9 aa, Artificial Sequence
  FAP(4B9)  CDR-L3
QQGIMLPPT

SEQ ID NO: 27 - protein, 117 aa, Artificial Sequence
  FAP(4B9)  VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 28 - protein, 108 aa, Artificial Sequence
  FAP(4B9)  VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 29 - protein, 5 aa, Artificial Sequence
  FAP (28H1) CDR-H1
SHAMS

SEQ ID NO: 30 - protein, 16 aa, Artificial Sequence
  FAP (28H1) CDR-H2
AIWASGEQYYADSVKG

SEQ ID NO: 31 - protein, 8 aa, Artificial Sequence
  FAP (28H1) CDR-H3
GWLGNFDY

SEQ ID NO: 32 - protein, 12 aa, Artificial Sequence
  FAP (28H1) CDR-L1
RASQSVSRSYLA

SEQ ID NO: 33 - protein, 7 aa, Artificial Sequence
  FAP (28H1) CDR-L2
GASTRAT

SEQ ID NO: 34 - protein, 9 aa, Artificial Sequence
  FAP (28H1) CDR-L3
QQGQVIPPT

SEQ ID NO: 35 - protein, 116 aa, Artificial Sequence
  FAP(28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 36 - protein, 108 aa, Artificial Sequence
  FAP(28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 37 - protein, 419 aa, Artificial Sequence
  Fc hole  huIgG1 PGLALA- 4-1BB lipocalin heavy chain
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 38 - protein, 639 aa, Artificial Sequence
  VH (FAP 4B9) Fc knob huIgG1 PGLALA 4-1BB lipocalin heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 39 - protein, 215 aa, Artificial Sequence
  VL (FAP 4B9) Ckappa light chain
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 10 aa, Artificial Sequence
  heavy chain CDR-H1, pertuzumab
GFTFTDYTMD

SEQ ID NO: 41 - protein, 17 aa, Artificial Sequence
  heavy chain CDR-H2, pertuzumab
DVNPNSGGSIYNQRFKG

SEQ ID NO: 42 - protein, 10 aa, Artificial Sequence
  heavy chain CDR-H3, pertuzumab
NLGPSFYFDY

SEQ ID NO: 43 - protein, 11 aa, Artificial Sequence
  light chain CDR-L1, pertuzumab
KASQDVSIGVA

SEQ ID NO: 44 - protein, 7 aa, Artificial Sequence
  Light chain CDR-L2, pertuzumab
SASYRYT

SEQ ID NO: 45 - protein, 9 aa, Artificial Sequence
  light chain CDR-L3, pertuzumab
QQYYIYPYT

SEQ ID NO: 46 - protein, 119 aa, Artificial Sequence [sequence omitted]

SEQ ID NO: 47 - protein, 107 aa, Artificial Sequence [sequence omitted]

SEQ ID NO: 48 - protein, 10 aa, Artificial Sequence
  heavy chain CDR-H1, trastuzumab
GFNIKDTYIH

SEQ ID NO: 49 - protein, 17 aa, Artificial Sequence
  heavy chain CDR-H2, trastuzumab
RIYPTNGYTRYADSVKG

SEQ ID NO: 50 - protein, 11 aa, Artificial Sequence
  heavy chain CDR-H3, trastuzumab
WGGDGFYAMDY

SEQ ID NO: 51 - protein, 11 aa, Artificial Sequence
  light chain CDR-L1, trastuzumab
RASQDVNTAVA

SEQ ID NO: 52 - protein, 7 aa, Artificial Sequence
  light chain CDR-L2, trastuzumab
SASFLYS

SEQ ID NO: 53 - protein, 9 aa, Artificial Sequence
  light chain CDR-L3, trastuzumab
QQHYTTPPT

SEQ ID NO: 54 - protein, 120 aa, Artificial Sequence [sequence omitted]

SEQ ID NO: 55 - protein, 107 aa, Artificial Sequence [sequence omitted]

SEQ ID NO: 56 - protein, 10 aa, Artificial Sequence
  heavy chain CDR-H1, aff. pertuzumab
GFTFNDYTMD

SEQ ID NO: 57 - protein, 17 aa, Artificial Sequence
  heavy chain CDR-H2, aff. pertuzumab
DVNPNSGGSIVNRRFKG

SEQ ID NO: 58 - protein, 10 aa, Artificial Sequence
  heavy chain CDR-H3, aff. pertuzumab
NLGPFFYFDY

SEQ ID NO: 59 - protein, 11 aa, Artificial Sequence
  light chain CDR-L1,aff.  pertuzumab
KASQDVSTAVA

SEQ ID NO: 60 - protein, 7 aa, Artificial Sequence
  light chain CDR-L2, aff. pertuzumab
SASFRYT

SEQ ID NO: 61 - protein, 9 aa, Artificial Sequence
  light chain CDR-L1,aff. pertuzumab
QQHYTTPPT

SEQ ID NO: 62 - protein, 119 aa, Artificial Sequence
  heavy chain variable domain VH, aff. Pertuzumab (aff-PER)
EVQLVESGGGLVQPGGSLRLSCAASGFTFNDYTMDWVRQAPGKGLEWVADVNPNSGGSIVNRRFKGRFTLSVDRSKNTLYLQMNSLRAEDTAVYYCARNLGPFFYFDYWGQGTLVTVSS

SEQ ID NO: 63 - protein, 107 aa, Artificial Sequence
  light chain variable domain VL, aff. Pertuzumab (aff-PER)
DIQMTQSPSSLSASVGDRVTITCKASQDVSTAVAWYQQKPGKAPKLLIYSASFRYTGVPSRFSGSRSGTDFTLTISSLQPEDFATYYCQQHYTTPPTFGQGTKVEIK

SEQ ID NO: 64 - protein, 419 aa, Artificial Sequence; identical to SEQ ID NO: 37
  Fc hole huIgG1 PGLALA - 4-1BB lipocalin heavy chain

SEQ ID NO: 65 - protein, 642 aa, Artificial Sequence
  VH (Her2 TRAS) Fc knob huIgG1 PGLALA 4-1BB lipocalin heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 66 - protein, 214 aa, Artificial Sequence
  VL (Her2 TRAS) Ckappa light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 639 aa, Artificial Sequence
  VH (FAP 4B9) - Fc huIgG1 PGLALA - 4-1BB lipocalin heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 68 - protein, 642 aa, Artificial Sequence
  VH (Her2 TRAS) - Fc huIgG1 PGLALA- 4-1BB lipocalin heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 69 - protein, 637 aa, Artificial Sequence
  VH (FAP 4B9) - Fc huIgG4 SP - 4-1BB lipocalin heavy chain
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 70 - protein, 215 aa, Artificial Sequence; identical to SEQ ID NO: 39
  VL (FAP 4B9) light chain

SEQ ID NO: 71 - protein, 635 aa, Artificial Sequence
  VH (DP47) - Fc huIgG4 SP - 4-1BB lipocalin heavy chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 72 - protein, 215 aa, Artificial Sequence
  VL (DP47) light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 640 aa, Artificial Sequence
  VH (Her2 TRAS) - Fc huIgG4 SP - 4-1BB lipocalin heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 74 - protein, 214 aa, Artificial Sequence; identical to SEQ ID NO: 66
  VL (Her2 TRAS) light chain

SEQ ID NO: 75 - protein, 5 aa, Artificial Sequence
  Peptide linker
GGGGS

SEQ ID NO: 76 - protein, 10 aa, Artificial Sequence
  Peptide linker
GGGGSGGGGS

SEQ ID NO: 77 - protein, 10 aa, Artificial Sequence
  Peptide linker
SGGGGSGGGG

SEQ ID NO: 78 - protein, 15 aa, Artificial Sequence
  Peptide linker
GGGGSGGGGSGGGGS

SEQ ID NO: 79 - protein, 14 aa, Artificial Sequence
  Peptide linker
GGGGSGGGGSGGGG

SEQ ID NO: 80 - protein, 20 aa, Artificial Sequence
  Peptide linker
GGGGSGGGGSGGGGSGGGGS

SEQ ID NO: 81 - protein, 10 aa, Artificial Sequence
  Peptide linker
GSPGSSSSGS

SEQ ID NO: 82 - protein, 8 aa, Artificial Sequence
  Peptide linker
GSGSGSGS

SEQ ID NO: 83 - protein, 8 aa, Artificial Sequence
  Peptide linker
GSGSGNGS

SEQ ID NO: 84 - protein, 8 aa, Artificial Sequence
  Peptide linker
GGSGSGSG

SEQ ID NO: 85 - protein, 6 aa, Artificial Sequence
  Peptide linker
GGSGSG

SEQ ID NO: 86 - protein, 4 aa, Artificial Sequence
  Peptide linker
GGSG

SEQ ID NO: 87 - protein, 8 aa, Artificial Sequence
  Peptide linker
GGSGNGSG

SEQ ID NO: 88 - protein, 8 aa, Artificial Sequence
  Peptide linker
GGNGSGSG

SEQ ID NO: 89 - protein, 6 aa, Artificial Sequence
  Peptide linker
GGNGSG

SEQ ID NO: 90 - protein, 152 aa, Homo sapiens
ASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPG

SEQ ID NO: 91 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 92 - protein, 748 aa, Artificial Sequence
  hu FAP ectodomain+poly-lys-tag+his6-tag
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 93 - protein, 762 aa, Mus musculus
MMKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 94 - protein, 749 aa, Artificial Sequence
  Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 95 - protein, 748 aa, Artificial Sequence
  Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 96 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 97 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 98 - protein, 1210 aa, Homo sapiens [sequence omitted]

SEQ ID NO: 99 - protein, 556 aa, Homo sapiens [sequence omitted]

SEQ ID NO: 100 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 101 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 102 - protein, 1245 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 103 - protein, 163 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 104 - protein, 163 aa, Cynomolgus
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQ

SEQ ID NO: 105 - protein, 164 aa, Mus musculus
VQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVL

SEQ ID NO: 106 - protein, 152 aa, Artificial Sequence
  Lipocalin mutein var.32
ASDEEIQDVSGTWYLKAMTVDEGCRPWNIFSVTPMTLTTLEGGNLEAKVTMAIDGPAQEVKAVLEKTDEPGKYTADGGKHVAYIIRSHVKDHYIFYSEGVCDGSPVPGVWLVGRDPKNNLEALEDFEKAAGARGLSTESILIPRQSETSSPG

SEQ ID NO: 107 - protein, 152 aa, Artificial Sequence
  Lipocalin mutein var.33
TSDEEIQDVSGTWYLKAMTVDEGCRPWNIFSVTPMTLTTLEGGNLEAKVTMAIDGPAQEVRAVLEKTDEPGKYTADGGKHDAYIIRSHVKDHYIFYSEGVCDGSPVPGVWLVGRDPENNLEALEDFEKTAGARGLSTESILIPRQSETSSPG

SEQ ID NO: 108 - protein, 152 aa, Artificial Sequence
  Lipocalin mutein var.34
ASDEEIQDVSGTWYLKAMTVDEGCRPWNIFSVTPMTLTTLEGGNLEAKVTMAIDGPAQEVNAVLEKTDEPGKYTADGGKHVAYIIRSHVRDHYIFYSEGVCDGSPVPGVWLVGRDPENNLEALEDFEKTAGARGLSTESILIPRQSETSSPG

SEQ ID NO: 109 - protein, 152 aa, Artificial Sequence
  Lipocalin mutein var.35
VSDEEIQDVSGTWYLKAMTVDEGCRPWNIFSVTPMTLTTLEGGNLEAKVTMAIDGPAQEVRAVLEKTDEPGKYTADGGKHVAYIIRSHVEDHYIFYSEGVCDGSPVPGVWLVGRDPENNLEALEDFEKTAGARGLSTESILIPRQSETSSPG

SEQ ID NO: 110 - protein, 152 aa, Artificial Sequence
  Lipocalin mutein var.36
ASDEEIQDVSGTWYLKAMTVDEGCRPWNIFSVTPMTLSTLEGGNLEAKVTMAIDGPAQEVKAVLEKTDEPGKYTADGGKHVAYIIRSHVKDHYIFYSEGVCDGSPVPGVWLVGRDPKNNLEALEDFEKAAGARGLSTESILIPRQIETSSPG

SEQ ID NO: 111 - protein, 152 aa, Artificial Sequence
  Lipocalin mutein var.37
ASDEEIQDVSGTWYLKAMTVDEGCRPWNIFSVTPMTLTTLEGGNLEAEVTMAIDGPAQEVKAVLEKADEPGKYTADGGKHVAYIIRSHVKDHYIFYSEGVCDGSPVPGVWLVGRDPKNNLEALEDFEKTAGARGLSTESILIPSQIETSSPG

SEQ ID NO: 112 - protein, 152 aa, Artificial Sequence
  Lipocalin mutein var.38
TSDEEIQDVSGTWYLKAMTVDEGCRPWNIFSVTPMTLTTLEDGNLEAKVTMAIDGPAQEVKAVLEKADEPGKYTADGGKHVAYIIRSHVKDHYIFYSEGVCDGSPVPGVWLVGRDPKNNLEALEDFEKAAGARGLSTESILIPRQIETSSPG

SEQ ID NO: 113 - protein, 16 aa, Artificial Sequence
  Peptide Linker
PSTPPTNSSSTIPTPS

SEQ ID NO: 114 - protein, 14 aa, Artificial Sequence
  Peptide Linker
GGSGNSSGSGGSPV

SEQ ID NO: 115 - protein, 17 aa, Artificial Sequence
  Peptide Linker
ASPAAPAPASPAAPAPA

SEQ ID NO: 116 - protein, 66 aa, Artificial Sequence
  Peptide Linker
AGSGGSGGSGGSPVPSTPPTPSPSTPPTPSPSGGSGNSSGSGGSPVPSTPPTPSPSTPPTPSPSAS

SEQ ID NO: 117 - protein, 32 aa, Artificial Sequence
  Peptide Linker
PSTPPTPSPSTPPTPSPSGGSGNSSGSGGSPV

SEQ ID NO: 118 - protein, 74 aa, Artificial Sequence
  Peptide Linker
AGSGGSGGSGGSPVPSTPPTNSSSTPPTPSPSPVPSTPPTNSSSTPPTPSPSPVPSTPPTNSSSTPPTPSPSAS

SEQ ID NO: 119 - protein, 40 aa, Artificial Sequence
  Peptide Linker
ASPAAPAPASPAAPAPSAPAASPAAPAPASPAAPAPSAPA

SEQ ID NO: 120 - protein, 10 aa, Artificial Sequence
  Peptide Linker
VDDIEGRMDE

SEQ ID NO: 121 - protein, 11 aa, Artificial Sequence
  Peptide Linker
ENLYFQGRMDE

SEQ ID NO: 122 - protein, 330 aa, Homo sapiens [sequence omitted]

SEQ ID NO: 123 - protein, 330 aa, Home sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 125 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 126 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 127 - protein, 327 aa, Artificial Sequence
  Fc huIgG4 SP
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 226 aa, Artificial Sequence
  Fc hole hu IgG1
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 129 - protein, 226 aa, Artificial Sequence
  Fc knob hu IgG1
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2022/0025069 A1
(43) Pub. Date: Jan. 27, 2022

(54) BISPECIFIC ANTIGEN BINDING MOLECULES COMPRISING LIPOCALIN MUTEINS

(71) Applicant: Hoffmann-La Roche Inc., Little Falls, NJ (US)

(72) Inventors: Christina CLAUS, Ennetbaden (CH); Claudia FERRARA KOLLER, Zug (CH); Christian KLEIN, Weilheim (DE); Pablo UMAÑA, Wollerau (CH)

(73) Assignee: Hoffmann-La Roche Inc., Little Falls, NJ (US)

(21) Appl. No.: 17/498,515

(22) Filed: Oct. 11, 2021

Related U.S. Application Data
(63) Continuation of application No. PCT/EP2020/059949, filed on Apr. 8, 2020.

(30) Foreign Application Priority Data
Apr. 12, 2019 (EP) 19169022.1

(51) Int. Cl. C07K 16/40; C07K 14/475; C07K 16/46; C07K 16/32

(52) U.S. Cl. CPC C07K 16/40; C07K 14/475; C07K 16/468; C07K 16/32; C07K 2319/33; C07K 2317/75; C07K 2317/55; C07K 2317/24; C07K 2317/31; C07K 2317/35; C07K 2317/92; C07K 2317/52

(57) ABSTRACT

The invention relates to bispecific antigen binding molecule capable of bivalent binding to 4-1BB and monovalent binding to a target cell antigen comprising two lipocalin muteins capable of specific binding to 4-1BB and their use in the treatment of cancer or infectious diseases.

### CROSS REFERENCE TO RELATED APPLICATIONS

This application is a continuation of International Patent Application No. PCT/EP2020/059949, filed Apr. 8, 2020, which claims priority to European Application Number 19169022.1 filed Apr. 12, 2019, which are incorporated herein by reference in its entirety.

### SEQUENCE LISTING

This application contains a Sequence Listing which has been submitted electronically in ASCII format and is hereby incorporated by reference in its entirety. Said ASCII copy, created on Oct. 8, 2021, is named P35474-US_Seq_listing_ST25.txt and is 241,127 bytes in size.

### TECHNICAL FIELD

The invention relates to bispecific antigen binding molecule capable of bivalent binding to 4-1BB and monovalent binding to a target cell antigen comprising two lipocalin muteins capable of specific binding to 4-1BB and their use in the treatment of cancer or infectious diseases. The invention further relates to methods of producing these molecules and to methods of using the same.

### BACKGROUND

4-1BB (CD137), a member of the TNF receptor superfamily, was first identified as an inducible molecule expressed by activated by T cells (Kwon and Weissman, 1989, Proc Natl Acad Sci USA 86, 1963-1967). Subsequent studies demonstrated that many other immune cells also express 4-1BB, including NK cells, B cells, NKT cells, monocytes, neutrophils, mast cells, dendritic cells (DCs) and cells of non-hematopoietic origin such as endothelial and smooth muscle cells (Vinay and Kwon, 2011, Cell Mol Immunol 8, 281-284). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., 2002, J Immunol 168, 3755-3762; Zhang et al., 2010, Clin Cancer Res 13, 2758-2767).

4-1BB ligand (4-1BBL or CD137L) was identified in 1993 (Goodwin et al., 1993, Eur J Immunol 23, 2631-2641). It has been shown that expression of 4-1BBL was restricted on professional antigen presenting cells (APC) such as B-cells, DCs and macrophages. Inducible expression of 4-1BBL is characteristic for T-cells, including both αβ and γδ T-cell subsets, and endothelial cells (Shao and Schwarz, 2011, J Leukoc Biol 89, 21-29).

Co-stimulation through the 4-1BB receptor (for example by 4-1BBL ligation) activates multiple signaling cascades within the T cell (both CD4⁺ and CD8⁺ subsets), powerfully augmenting T cell activation (Bartkowiak and Curran, 2015, Front Oncol 5, 117). In combination with TCR triggering, agonistic 4-1BB-specific antibodies enhance proliferation of T-cells, stimulate lymphokine secretion and decrease sensitivity of T-lymphocytes to activation-induced cells death (Snell et al., 2011, Immunol Rev 244, 197-217). This mechanism was further advanced as the first proof of concept in cancer immunotherapy. In a preclinical model administration of an agonistic antibody against 4-1BB in tumor bearing mice led to potent anti-tumor effect (Melero et al., 1997, Nat Med 3, 682-685). Later, accumulating evidence indicated that 4-1BB usually exhibits its potency as an anti-tumor agent only when administered in combination with other immunomodulatory compounds, chemotherapeutic reagents, tumor-specific vaccination or radiotherapy (Bartkowiak and Curran, 2015, Front Oncol 5, 117).

Signaling of the TNFR-superfamily needs cross-linking of the trimerized ligands to engage with the receptors, so does the 4-1BB agonistic antibodies which require wild type Fc-binding (Li and Ravetch, 2011, Science 333, 1030-1034). However, systemic administration of 4-1BB-specific agonistic antibodies with the functionally active Fc domain resulted in influx of CD8⁺ T-cells associated with liver toxicity (Dubrot et al., 2010, Cancer Immunol Immunother 59, 1223-1233) that is diminished or significantly ameliorated in the absence of functional Fc-receptors in mice. In the clinic, an Fc-competent 4-1BB agonistic Ab (BMS-663513) (NCT00612664) caused a grade 4 hepatitis leading to termination of the trial (Simeone and Ascierto, 2012, J Immunotoxicol 9, 241-247). Therefore, there is a need for effective and safer 4-1BB agonists.

Human Fibroblast Activation Protein (FAP; GenBank Accession Number AAC51668), also known as Seprase, is a 170 kDa integral membrane serine peptidase (EC 3.4.21.B28). Together with dipeptidyl peptidase IV (also known as CD26; GenBank Accession Number P27487), a closely related cell-surface enzyme, and other peptidases, FAP belongs to the dipeptidyl peptidase IV family (Yu et al., FEBS J 277, 1126-1144 (2010)). It is a homodimer containing two N-glycosylated subunits with a large C-terminal extracellular domain, in which the enzyme's catalytic domain is located (Scanlan et al., Proc Natl Acad Sci USA 91, 5657-5661 (1994)). FAP, in its glycosylated form, has both post-prolyl dipeptidyl peptidase and gelatinase activities (Sun et al., Protein Expr Purif 24, 274-281 (2002)). Due to its expression in many common cancers and its restricted expression in normal tissues, FAP has been considered a promising antigenic target for imaging, diagnosis and therapy of a variety of carcinomas. Thus, multiple monoclonal antibodies have been raised against FAP for research, diagnostic and therapeutic purposes.

The human epidermal growth factor receptor-2 (HER2; ErbB2) is a receptor tyrosine kinase and a member of the epidermal growth factor receptor (EGFR) family of transmembrane receptors. HER2 is overexpressed in a range of tumor types and it has been implicated in disease initiation and progression. It is associated with poor prognosis. For example, overexpression of HER2 is observed in approximately 30% of human breast cancers and it is implicated in the aggressive growth and poor clinical outcomes associated with these tumors (Slamon et al (1987) Science 235:177-182).

The humanized anti-HER2 monoclonal antibody trastuzumab (CAS 180288-69-1, HERCEPTIN®, huMAb4D5-8, rhuMAb HER2, Genentech) targets the extracellular domain of HER2 (U.S. Pat. Nos. 5,677,171; 5,821,337; 6,054,297; 6,165,464; 6,339,142; 6,407,213; U.S. Pat. Nos. 6,639,055; 6,719,971; 6,800,738; 7,074,404; Coussens et al (1985) Science 230:1 132-9; Slamon et al (1989) Science 244:707-12; Slamon et al (2001) New Engl. J. Med. 344:783-792). Trastuzumab has been shown to inhibit the proliferation of human tumor cells that overexpress HER2 and is a mediator of antibody-dependent cellular cytotoxicity, ADCC (Hudziak et al (1989) Mol Cell Biol 9:1 165-72; Lewis et al (1993) Cancer Immunol Immunother; 37:255-63; Baselga et al (1998) Cancer Res. 58:2825-2831; Hotaling et al (1996) [abstract]. Proc. Annual Meeting Am Assoc Cancer Res; 37:471; Pegram M D, et al (1997) [abstract]. Proc Am Assoc Cancer Res; 38:602; Sliwkowski et al (1999) Seminars in Oncology 26(4), Suppl 12:60-70; Yarden Y. and Sliwkowski, M. (2001) Nature Reviews: Molecular Cell Biology, Macmillan Magazines, Ltd., Vol. 2:127-137).

HERCEPTIN® (trastuzumab, Genentech Inc.) was approved in 1998 for the treatment of of patients with HER2-overexpressing metastatic breast cancers (Baselga et al, (1996) J. Clin. Oncol. 14:737-744). In 2006, the FDA approved HERCEPTIN® as part of a treatment regimen containing doxorubicin, cyclophosphamide and paclitaxel for the adjuvant treatment of patients with HER2-positive, node-positive breast cancer.

Pertuzumab (also known as recombinant humanized monoclonal antibody 2C4, rhuMAb 2C4, PERJETA®, Genentech, Inc, South San Francisco) is another antibody treatment targeting HER2. Pertuzumab is a Her dimerization inhibitor (HDI) and functions to inhibit the ability of HER2 to form active heterodimers or homodimers with other Her receptors (such as EGFR/HER1, HER2, HER3 and HER4). See, for example, Harari and Yarden Oncogene 19:6102-14 (2000); Yarden and Sliwkowski. Nat Rev Mol Cell Biol 2:127-37 (2001); Sliwkowski, Nat Struct Biol 10:158-9 (2003); Cho et al. Nature 421:756-60 (2003); and Malik et al., Pro Am Soc Cancer Res 44:176-7 (2003); U.S. Pat. No. 7,560,111. PERJETA® was first approved in 2012 in combination with trastuzumab and docetaxel for the treatment of patients with advanced or late-stage (metastatic) HER2-positive breast cancer. The combination therapy using trastuzumab and pertuzumab is meanwhile also approved for the neoadjuvant (before surgery) treatment of HER2-positive, locally advanced, inflammatory, or early stage breast cancer and for adjuvant (after surgery) treatment of HER2-positive early breast cancer (EBC) at high risk of recurrence. The mechanisms of action of Perjeta and Herceptin are believed to complement each other, as both bind to the HER2 receptor, but to different places. The combination of Perjeta and Herceptin is thought to provide a more comprehensive, dual blockade of HER signaling pathways, thus preventing tumor cell growth and survival.

Bispecific, bivalent HER2 antibodies that are directed against domains II, III and IV of human ErbB2 are disclosed in WO 2012/143523. Bispecific HER-2 antibodies comprising optimized variants of the antibodies rhuMab 2C4 and hu4D5, called Herceptarg, have been described in WO 2015/091738. Although the therapeutic efficacy of trastuzumab in breast carcinoma is well demonstrated, there are many patients who do not benefit from trastuzumab because of resistance. Given the lack of an effective anti-HER2 therapy in specific cancers expressing low levels of HER2, the resistance to the current therapies, and the prevalence of HER2 related cancers, new therapies are required to treat such cancers.

The bispecific antigen binding molecules of the present invention are characterized by their binding against a target cell antigen, in particular a tumor target such as FAP or HER2, and their binding specificity for 4-1BB. The antigen binding domains capable of specific binding to 4-1BB are represented by lipocalin muteins. Lipocalin muteins (anticalins) are non-antibody scaffolds derived from natural human lipocalins and provide several benefits such as small size, robust fold and pronounced target specificity (Rothe C, Skerra A., BioDrugs 2018, 32, 233-243). Lipocalin muteins specific for CD137 (4-1BB) are described in WO 2016/177762 and WO 2018/087108. Fusion proteins composed of a binding specificity for CD137 and a binding specificity for HER2/neu are disclosed in WO 2016/177802. Based on their Fc domain these fusion proteins form symmetric antibody-like dimers with bivalent binding to CD137 and to HER2.

The binding antigen binding molecules of the present invention are characterized in that they provide monovalent binding to the target cell antigen and bivalent binding to 4-1BB. Surprisingly, it has been found that a ratio of 1:2 of tumor-target-binding to effector-cell-target-binding leads to improved crosslinking of 4-1BB agonist on the effector cells, a stronger 4-1BB receptor downstream signaling and thus improved efficacy.

### SUMMARY

In one aspect, the invention provides a bispecific antigen binding molecule capable of bivalent binding to 4-1BB and monovalent binding to a target cell antigen, comprising

(a) an antigen binding domain capable of specific binding to a target cell antigen,
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

In a particular aspect, the invention provides a bispecific antigen binding molecule, wherein each of the lipocalin muteins capable of specific binding to 4-1BB is a lipocalin mutein derived from mature human neutrophil gelatinase-associated lipocalin (huNGAL) of SEQ ID NO:1.

In a further aspect, the invention provides a bispecific antigen binding molecule as defined above, wherein each of the lipocalin muteins capable of specific binding to 4-1BB comprise the amino acid sequence of SEQ ID NO:2 or an amino acid sequence of SEQ ID NO:2, wherein one or more of the following amino acids are mutated as following:

(a) Q at position 20 is replaced by R, or
(b) N at position 25 is replaced by Y or D, or
(c) H at position 28 is replaced by Q, or
(d) Q at position 36 is replaced by M, or
(e) I at position 40 is replaced by N, or
(f) R at position 41 is replaced by L or K, or
(g) E at position 44 is replaced by V or D, or
(h) K at position 46 is replaced by S and the amino acids at positions 47 to 49 are deleted, or
(i) I at position 49 is replaced by H, N, V or S, or
(j) M at position 52 is replaced by S or G, or
(k) K at position 59 is replaced by N, or
(l) D at position 65 is replaced by N, or
(m) M at position 68 is replaced by D, G or A, or
(n) K at position 70 is replaced by M, T, A or S, or
(o) F at position 71 is replaced by L, or
(p) D at position 72 is replaced by L, or
(q) M at position 77 is replaced by Q, H, T, R or N, or
(s) D at position 79 is replaced by I or A, or
(t) I at position 80 is replaced by N, or
(u) W at position 81 is replaced by Q, S or M, or
(v) T at position 82 is replaced by P, or
(w) F at position 83 is replaced by L, or
(y) F at position 92 is replaced by L or S, or
(z) L at position 94 is replaced by F, or
(za) K at position 96 is replaced by F, or
(zb) F at position 100 is replaced by D, or
(zc) P at position 101 is replaced by L, or
(zd) H at position 103 is replaced by P, or
(ze) S at position 106 is replaced by Y, or
(zf) F at position 122 is replaced by Y, or
(zg) F at position 125 is replaced by S, or
(zh) F at position 127 it replaced by I, or
(zi) E at position 132 is replaced by W, or
(zj) Y at position 134 is replaced by G.

In one aspect, the lipocalin muteins capable of specific binding to 4-1BB comprise an amino acid sequence of SEQ ID NO:2, wherein 4 to 10 amino acids have been mutated as defined above. In one aspect, each of the lipocalin muteins capable of specific binding to 4-1BB comprise an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In one aspect, each of the lipocalin muteins capable of specific binding to 4-1BB comprise an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10. In a further aspect, each of the lipocalin muteins capable of specific binding to 4-1BB comprise an amino acid sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In one aspect, each of the lipocalin muteins capable of specific binding to 4-1BB comprise the amino acid sequence of SEQ ID NO:2. In one aspect, both lipocalin muteins comprise an identical amino acid sequence.

In one aspect, the Fc domain is an IgG, particularly an IgG1 Fc domain or an IgG4 Fc domain. More particularly, the Fc domain is an IgG1 Fc domain. In a particular aspect, the Fc domain comprises a modification promoting the association of the first and second subunit of the Fc domain. In a particular aspect, provided is a bispecific antigen binding molecule, wherein the Fc domain comprises knob-into-hole modifications promoting association of the first and the second subunit of the Fc domain. In a specific aspect, provided is a bispecific antigen binding molecule, wherein the first subunit of the Fc domain comprises the amino acid substitutions S354C and T366W (EU numbering according to Kabat) and the second subunit of the Fc domain comprises the amino acid substitutions Y349C, T366S, L368A and Y407V (EU numbering according to Kabat).

In another aspect, the invention is concerned with a bispecific antigen binding molecule as defined herein before, comprising (b) a Fc domain composed of a first and a second subunit capable of stable association, wherein the Fc domain comprises one or more amino acid substitution that reduces binding to an Fc receptor, in particular towards Fcγ receptor. In particular, the Fc domain comprises amino acid substitutions at positions 234 and 235 (EU numbering according to Kabat) and/or 329 (EU numbering according to Kabat) of the IgG heavy chains. Particularly, provided is a bispecific antigen binding molecule, wherein the Fc domain is a human IgG1 Fc domain comprising the amino acid substitutions the amino acid substitutions L234A, L235A and P329G (EU numbering according to Kabat). In a further aspect, provided is a bispecific antigen binding molecule, wherein the Fc domain is a human IgG4 Fc domain comprising one or more amino acid substitutions selected from the group consisting of S228P, N297A, F234A and L235A (EU numbering according to Kabat), in particular the amino acid substitution S228P, F234A and L235A (EU numbering according to Kabat), more particularly the amino acid substitution S228P (EU numbering according to Kabat).

In one aspect, the invention provides a bispecific antigen binding molecule comprising two lipocalin muteins capable of specific binding to 4-1BB, wherein one of the lipocalin muteins is fused to the C-terminus of the first subunit of the Fc domain via a peptide linker and the other is fused to the C-terminus of the second subunit of the Fc domain via a peptide linker. In one aspect, the peptide linker has an amino acid sequence selected from the group consisting of SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:113, SEQ ID NO:114, SEQ ID NO:115, SEQ ID NO:116, SEQ ID NO: 117, SEQ ID NO:118, SEQ ID NO:119, SEQ ID NO:120 and SEQ ID NO:121. In one aspect, the peptide linker has an amino acid sequence selected from the group consisting of SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88 and SEQ ID NO:89. In particular, the peptide linker has the amino acid sequence of SEQ ID NO:78, i.e. (G₄S)₃.

In one particular aspect, the invention provides a bispecific antigen binding molecule capable of bivalent binding to 4-1BB and monovalent binding to a target cell antigen, wherein the antigen binding domain capable of specific binding to a target cell antigen is a Fab fragment capable of specific binding to a target cell antigen. Thus, the invention provides a bispecific antigen binding molecule comprising

(a) a Fab fragment capable of specific binding to a target cell antigen,
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

In one aspect, provided is a bispecific antigen binding molecule capable of bivalent binding to 4-1BB and monovalent binding to a target cell antigen, wherein the target cell antigen is Fibroblast Activation Protein (FAP). Thus, provided is a bispecific antigen binding molecule as defined above, wherein the Fab fragment capable of specific binding to a target cell antigen is a Fab fragment capable of specific binding to Fibroblast Activation Protein (FAP).

In one aspect, the Fab fragment capable of specific binding to Fibroblast Activation Protein (FAP) comprises (a) a heavy chain variable region (V_{H}FAP) comprising (i) CDR-H1 comprising the amino acid sequence of SEQ ID NO:21, (ii) CDR-H2 comprising the amino acid sequence of SEQ ID NO:22, and (iii) CDR-H3 comprising the amino acid sequence of SEQ ID NO:23, and a light chain variable region (V_{L}FAP) comprising (iv) CDR-L1 comprising the amino acid sequence of SEQ ID NO:24, (v) CDR-L2 comprising the amino acid sequence of SEQ ID NO:25, and (vi) CDR-L3 comprising the amino acid sequence of SEQ ID NO:26, or (b) a heavy chain variable region (V_{H}FAP) comprising (i) CDR-H1 comprising the amino acid sequence of SEQ ID NO:29, (ii) CDR-H2 comprising the amino acid sequence of SEQ ID NO:30, and (iii) CDR-H3 comprising the amino acid sequence of SEQ ID NO:31, and a light chain variable region (V_{L}FAP) comprising (iv) CDR-L1 comprising the amino acid sequence of SEQ ID NO:32, (v) CDR-L2 comprising the amino acid sequence of SEQ ID NO:33, and (vi) CDR-L3 comprising the amino acid sequence of SEQ ID NO:34. Particularly, the Fab fragment capable of specific binding to FAP comprises a heavy chain variable region (V_{H}FAP) comprising (i) CDR-H1 comprising the amino acid sequence of SEQ ID NO:21, (ii) CDR-H2 comprising the amino acid sequence of SEQ ID NO:22, and (iii) CDR-H3 comprising the amino acid sequence of SEQ ID NO:23, and a light chain variable region (V_{L}FAP) comprising (iv) CDR-L1 comprising the amino acid sequence of SEQ ID NO:24, (v) CDR-L2 comprising the amino acid sequence of SEQ ID NO:25, and (vi) CDR-L3 comprising the amino acid sequence of SEQ ID NO:26.

In one aspect, the Fab fragment capable of specific binding to Fibroblast Activation Protein (FAP) comprises (a) a heavy chain variable region (V_{H}FAP) comprising an amino acid sequence that is at least about 95%, 96%, 97%, 98%, 99% or 100% identical to the amino acid sequence of SEQ ID NO:27, and a light chain variable region (V_{L}FAP) comprising an amino acid sequence that is at least about 95%, 96%, 97%, 98%, 99% or 100% identical to the amino acid sequence of SEQ ID NO:28, or (b) a heavy chain variable region (V_{H}FAP) comprising an amino acid sequence that is at least about 95%, 96%, 97%, 98%, 99% or 100% identical to the amino acid sequence of SEQ ID NO:35, and a light chain variable region (V_{L}FAP) comprising an amino acid sequence that is at least about 95%, 96%, 97%, 98%, 99% or 100% identical to the amino acid sequence of SEQ ID NO:36. In particular, the Fab fragment capable of specific binding to FAP comprises a heavy chain variable region (V_{H}FAP) comprising the amino acid sequence of SEQ ID NO:27 and a light chain variable region (V_{L}FAP) comprising the amino acid sequence of SEQ ID NO:28, or (b) a heavy chain variable region (V_{H}FAP) comprising the amino acid sequence of SEQ ID NO:35 and a light chain variable region (V_{L}FAP) comprising the amino acid sequence of SEQ ID NO:36. More particularly, the Fab fragment capable of specific binding to FAP comprises a heavy chain variable region (V_{H}FAP) comprising the amino acid sequence of SEQ ID NO:27 and a light chain variable region (V_{L}FAP) comprising the amino acid sequence of SEQ ID NO:28.

In one aspect, the invention provides a bispecific antigen binding molecule capable of bivalent binding to 4-1BB and monovalent binding to FAP comprising a first heavy chain of SEQ ID NO:37, a second heavy chain of SEQ ID NO:38 and a light chain of SEQ ID NO:39.

In another aspect, provided is a bispecific antigen binding molecule capable of bivalent binding to 4-1BB and monovalent binding to a target cell antigen, wherein the target cell antigen is HER2. Thus, provided is a bispecific antigen binding molecule as defined above, wherein the Fab fragment capable of specific binding to a target cell antigen is a Fab fragment capable of specific binding to HER2.

In one aspect, the Fab fragment capable of specific binding to HER2 comprises

(a) a VH domain comprising (i) CDR-H1 comprising the amino acid sequence of SEQ ID NO:40, (ii) CDR-H2 comprising the amino acid sequence of SEQ ID NO:41, and (iii) CDR-H3 comprising the amino acid sequence of SEQ ID NO:42, and a VL domain comprising (iv) CDR-L1 comprising the amino acid sequence of SEQ ID NO:43, (v) CDR-L2 comprising the amino acid sequence of SEQ ID NO:44, and (vi) CDR-L3 comprising the amino acid sequence of SEQ ID NO:45, or (b) a VH domain comprising (i) CDR-H1 comprising the amino acid sequence of SEQ ID NO:48, (ii) CDR-H2 comprising the amino acid sequence of SEQ ID NO:49, and (iii) CDR-H3 comprising the amino acid sequence of SEQ ID NO:50, and a VL domain comprising (iv) CDR-L1 comprising the amino acid sequence of SEQ ID NO:51, (v) CDR-L2 comprising the amino acid sequence of SEQ ID NO:52, and (vi) CDR-L3 comprising the amino acid sequence of SEQ ID NO:53, or (c) a VH domain comprising (i) CDR-H1 comprising the amino acid sequence of SEQ ID NO:56, (ii) CDR-H2 comprising the amino acid sequence of SEQ ID NO:57, and (iii) CDR-H3 comprising the amino acid sequence of SEQ ID NO:58, and a VL domain comprising (iv) CDR-L1 comprising the amino acid sequence of SEQ ID NO:59, (v) CDR-L2 comprising the amino acid sequence of SEQ ID NO:60, and (vi) CDR-L3 comprising the amino acid sequence of SEQ ID NO:61. In one aspect, the Fab fragment capable of specific binding to HER2 comprises a VH domain comprising (i) CDR-H1 comprising the amino acid sequence of SEQ ID NO:40, (ii) CDR-H2 comprising the amino acid sequence of SEQ ID NO:41, and (iii) CDR-H3 comprising the amino acid sequence of SEQ ID NO:42, and a VL domain comprising (iv) CDR-L1 comprising the amino acid sequence of SEQ ID NO:43, (v) CDR-L2 comprising the amino acid sequence of SEQ ID NO:44, and (vi) CDR-L3 comprising the amino acid sequence of SEQ ID NO:45. In one aspect, the Fab fragment capable of specific binding to HER2 comprises a VH domain comprising (i) CDR-H1 comprising the amino acid sequence of SEQ ID NO:48, (ii) CDR-H2 comprising the amino acid sequence of SEQ ID NO:49, and (iii) CDR-H3 comprising the amino acid sequence of SEQ ID NO:50, and a VL domain comprising (iv) CDR-L1 comprising the amino acid sequence of SEQ ID NO:51, (v) CDR-L2 comprising the amino acid sequence of SEQ ID NO:52, and (vi) CDR-L3 comprising the amino acid sequence of SEQ ID NO:53.

In one aspect, the Fab fragment capable of specific binding to HER2 comprises (a) a heavy chain variable region (V_{H}HER2) comprising an amino acid sequence that is at least about 95%, 96%, 97%, 98%, 99% or 100% identical to the amino acid sequence of SEQ ID NO:46, and a light chain variable region (V_{L}HER2) comprising an amino acid sequence that is at least about 95%, 96%, 97%, 98%, 99% or 100% identical to the amino acid sequence of SEQ ID NO:47, or (b) a heavy chain variable region (V_{H}HER2) comprising an amino acid sequence that is at least about 95%, 96%, 97%, 98%, 99% or 100% identical to the amino acid sequence of SEQ ID NO:54, and a light chain variable region (V_{L}HER2) comprising an amino acid sequence that is at least about 95%, 96%, 97%, 98%, 99% or 100% identical to the amino acid sequence of SEQ ID NO:55, or (c) a heavy chain variable region (V_{H}HER2) comprising an amino acid sequence that is at least about 95%, 96%, 97%, 98%, 99% or 100% identical to the amino acid sequence of SEQ ID NO:62, and a light chain variable region (V_{L}HER2) comprising an amino acid sequence that is at least about 95%, 96%, 97%, 98%, 99% or 100% identical to the amino acid sequence of SEQ ID NO:63. In one aspect, the Fab fragment capable of specific binding to HER2 comprises (a) a heavy chain variable region (V_{H}HER2) comprising the amino acid sequence of SEQ ID NO:46 and a light chain variable region (V_{L}HER2) comprising the amino acid sequence of SEQ ID NO:47, or (b) a heavy chain variable region (V_{H}HER2) comprising the amino acid sequence of SEQ ID NO:54 and a light chain variable region (V_{L}HER2) comprising the amino acid sequence of SEQ ID NO:55, or

(c) a heavy chain variable region (V_{H}HER2) comprising the amino acid sequence of SEQ ID NO:62 and a light chain variable region (V_{L}HER2) comprising the amino acid sequence of SEQ ID NO:63. In one particular aspect, the Fab fragment capable of specific binding to HER2 comprises a heavy chain variable region (V_{H}HER2) comprising the amino acid sequence of SEQ ID NO:46 and a light chain variable region (V_{L}HER2) comprising the amino acid sequence of SEQ ID NO:47. In one aspect, the Fab fragment capable of specific binding to HER2 comprises a heavy chain variable region (V_{H}HER2) comprising the amino acid sequence of SEQ ID NO:54 and a light chain variable region (V_{L}HER2) comprising the amino acid sequence of SEQ ID NO:55.

In one aspect, the invention provides a bispecific antigen binding molecule capable of bivalent binding to 4-1BB and monovalent binding to HER2 comprising a first heavy chain of SEQ ID NO:64, a second heavy chain of SEQ ID NO:65 and a light chain of SEQ ID NO:66.

According to another aspect of the invention, there is provided isolated nucleic acid encoding a bispecific antigen binding molecule as defined herein before. The invention further provides a vector, particularly an expression vector, comprising the isolated nucleic acid of the invention and a host cell comprising the isolated nucleic acid or the vector of the invention. In some embodiments the host cell is a eukaryotic cell, particularly a mammalian cell.

In another aspect, provided is a method for producing the bispecific antigen binding molecule of the invention, comprising culturing the host cell of the invention under conditions suitable for expression of the bispecific antigen binding molecule, and further comprising recovering the bispecific antigen binding molecule from the host cell. The invention also encompasses a bispecific antigen binding molecule produced by the method of the invention.

Further provided is a pharmaceutical composition comprising the bispecific antigen binding molecule of the invention and at least one pharmaceutically acceptable excipient. In another aspect, a pharmaceutical composition is provided comprising the bispecific antigen binding molecule of the invention and at least one pharmaceutically acceptable excipient, further comprising an additional therapeutic agent, e.g. a chemotherapeutic agent and/or other agents for use in cancer immunotherapy.

Also encompassed by the invention is the bispecific antigen binding molecule of the invention, or the pharmaceutical composition of the invention, for use as a medicament. In one aspect is provided the bispecific antigen binding molecule of the invention, or the pharmaceutical composition of the invention, for use in the treatment of a disease in an individual in need thereof. In a specific aspect, provided is the bispecific antigen binding molecule of the invention, or the pharmaceutical composition of the invention, for use in the treatment of cancer or an infectious disease. In another aspect, provided is the bispecific antigen binding molecule of the invention, or the pharmaceutical composition of the invention, for use in up-regulating or prolonging cytotoxic T cell activity.

Also provided is the use of the bispecific antigen binding molecule of the invention for the manufacture of a medicament for the treatment of a disease in an individual in need thereof, in particular for the manufacture of a medicament for the treatment of cancer or an infectious disease, as well as a method of treating a disease in an individual, comprising administering to said individual a therapeutically effective amount of a composition comprising the bispecific antigen binding molecule as disclosed herein in a pharmaceutically acceptable form. In one aspect, the disease is cancer or an infectious disease. In a specific aspect, the disease is cancer. Also provided is a method of up-regulating or prolonging cytotoxic T cell activity in an individual having cancer, comprising administering to the individual an effective amount of the bispecific antigen binding molecule of the invention, or the pharmaceutical composition of the invention. In any of the above embodiments the individual is preferably a mammal, particularly a human.

### BRIEF DESCRIPTION OF THE DRAWINGS

FIGS. 1A and 1B show the bispecific antigen binding molecules comprising two fusion proteins capable of specific binding to 4-1BB targeted to tumor antigen (TA). In FIG. 1A a bispecific antigen binding molecule that is bivalent for both the tumor target antigen (TA1) and for 4-1BB, termed also 2+2 format. In FIG. 1B a bispecific antigen binding molecule of the invention is shown that is monovalent for TA1 and bivalent for 4-1BB, termed also 1+2 format. Both antigen binding molecules are in huIgG1 P329GLALA format.

FIG. 2A shows the setup of the SPR experiments for simultaneous binding of the FAP-targeting bispecific antigen binding molecules comprising two fusion proteins capable of specific binding to 4-1BB (TA1 is FAP). In FIGS. 2B and 2C the simultaneous binding of the bispecific anti-FAP, anti-4-1BB lipocalin huIgG1 PGLALA antigen binding molecule (Analyte 1) to immobilized human 4-1BB and human FAP (Analyte 2) is shown. The simultaneous binding of bispecific, bivalent 2+2 anti-FAP, anti-4-1BB lipocalin huIgG1 PGLALA (termed 2+2) is shown in FIG. 2B. Simultaneous binding to human 4-1BB and human FAP of bispecific, monovalent 1+2 anti-FAP, anti-4-1BB lipocalin huIgG1 PGLALA (termed 1+2) is shown in FIG. 2C.

FIG. 3A shows the setup of the SPR experiments for simultaneous binding of the HER2-targeting bispecific 4-1BB lipocalins (TA1 is HER2). In FIG. 3B the simultaneous binding of the bispecific anti-HER2, anti-4-1BB huIgG1 PGLALA antigen binding molecules in 2+2 and 1+2 format (Analyte 1) to immobilized human 4-1BB and human HER2 (Analyte 2) is shown.

FIG. 4 shows the binding of FAP-targeting 4-1BB lipocalins to FAP expressed on human FAP-expressing cell line NIH/3T3-huFAP clone 19 cells. The concentration of bispecific, bivalent 2+2 anti-FAP, anti-4-1BB lipocalin huIgG1 PGLALA (termed FAP (4B9)×4-1BB lipocalin huIgG1 PG LALA 2+2, open down-facing triangle and dotted line) or bispecific, monovalent 1+2 anti-FAP, anti-4-1BB lipocalin huIgG1 PGLALA (termed FAP (4B9)×4-1BB lipocalin huIgG1 PG LALA 1+2, filled black triangle and line) or its controls is blotted against the geo mean of fluorescence intensity (gMFI) of the PE-conjugated secondary detection antibody. All values are baseline corrected by subtracting the baseline values of the blank control (e.g. no primary only secondary detection antibody). Only FAP-binding-domain-containing constructs like FAP (4B9)×4-1BB lipocalin huIgG1 PG LALA 2+2 (open down-facing triangle and dotted line), FAP (4B9)×4-1BB lipocalin huIgG1 PG LALA 1+2 (filled black triangle and line), FAP (4B9)×4-1BB lipocalin huIgG4 SP 2+2 (half filled black circle and line-dotted line) or the FAP (4B9) huIgG1 PG LALA antibody (grey star and line) bind efficiently to FAP-expressing cells.

FIG. 5 illustrates the binding of FAP-targeting 4-1BB lipocalins to human 4-1BB (CD137) expressing reporter cell line Jurkat-hu4-1BB-NFkB-luc2. The concentration of bispecific, bivalent 2+2 anti-FAP, anti-4-1BB lipocalin huIgG1 PGLALA (termed FAP (4B9)×4-1BB lipocalin huIgG1 PG LALA 2+2, open down-facing triangle and dotted line) or bispecific, monovalent 1+2 anti-FAP, anti-4-1BB lipocalin huIgG1 PGLALA (termed FAP (4B9)×4-1BB lipocalin huIgG1 PG LALA 1+2, filled black triangle and line) or its controls is blotted against the geo mean of fluorescence intensity (gMFI) of the PE-conjugated secondary detection antibody. All values are baseline corrected by subtracting the baseline values of the blank control (e.g. no primary only secondary detection antibody). Anti-4-1BB (20H4.9)×anti-FAP (4B9) 2+1 H2H binds (black filled circle and line) similar to 4-1BB as its control anti-4-1BB (20H4.9) huIgG1 P329G LALA (grey star and line).

The activation of the NFκB signaling pathway by measuring the NFκB-mediated luciferase activity in a Jurkat-hu4-1BB-NFκB-luc2 reporter cell line is shown in FIGS. 6A to 6C. To test the functionality of bispecific, bivalent 2+2 anti-FAP, anti-4-1BB lipocalin huIgG1 PGLALA (termed FAP (4B9)×4-1BB lipocalin huIgG1 PG LALA 2+2, open, facing-down black triangle and dotted line) or bispecific, monovalent 1+2 anti-FAP, anti-4-1BB lipocalin huIgG1 PGLALA (termed FAP (4B9)×4-1BB lipocalin huIgG1 PG LALA 1+2, filled black triangle and line) or the control molecule bispecific, bivalent 2+2 anti-FAP, anti-4-1BB lipocalin huIgG4 PGLALA (termed FAP (4B9)×4-1BB lipocalin huIgG4 SP 2+2, half-filled black hexamer, and line-dotted line) or monospecific control molecules were incubated at different titrated concentrations with the reporter cell line Jurkat-hu4-1BB-NFκB-luc2 in the absence or presence of FAP-expressing cell lines WM-266-4 or NIH/3T3-huFAP clone 19. All molecules failed to activate 4-1BB signaling in the absence of FAP-expressing cells, as no crosslinking occurs. In the presence of FAP-expressing cells only bispecific molecules binding FAP and 4-1BB lead to NFκB activation on the reporter cell line. The results in the absence of FAP+ cells are shown in FIG. 6A, in the presence of human FAP expressing cell line WM-266-4 in FIG. 6B or in the presence of human FAP expressing cell line NIH/3T3-huFAP clone 19 in FIG. 6C.

FIGS. 7A and 7B show the binding of HER2-targeting 4-1BB lipocalins to HER2 expressed on the cell surface by human gastric carcinoma cell line NCI-N87 (FIG. 7B) or breast adenocarcinoma cell line KPL4 (FIG. 7A). The concentration of bispecific, bivalent 2+2 anti-HER2, anti-4-1BB lipocalin huIgG1 PGLALA (termed HER2 (TRAS)×4-1BB lipocalin huIgG1 PG LALA 2+2, black open down-facing triangle, dotted line) or bispecific, monovalent 1+2 anti-HER2, anti-4-1BB lipocalin huIgG1 PGLALA (termed HER2 (TRAS)×4-1BB lipocalin huIgG1 PG LALA 1+2, black filled triangle and line) or its controls is blotted against the geo mean of fluorescence intensity (gMFI) of the PE-conjugated secondary detection antibody. All values are baseline corrected by subtracting the baseline values of the blank control (e.g. no primary only secondary detection antibody). Only HER2-binding-domain-containing constructs like, bivalent 2+2 anti-HER2, anti-4-1BB huIgG1 PGLALA (termed HER2 (TRAS)×4-1BB lipocalin huIgG1 PG LALA 2+2, black open down-facing triangle, dotted line) or bispecific, monovalent 1+2 anti-HER2, anti-4-1BB lipocalin huIgG1 PGLALA (termed HER2 (TRAS)×4-1BB lipocalin huIgG1 PG LALA 1+2, black filled triangle and line) or the HER2 (TRAS) huIgG1 PG LALA antibody (grey star and line) or HER2 (TRAS)×4-1BB lipocalin huIgG4 SP (half-filled black hexamer, black dotted line) bind efficiently to HER2-expressing cells.

FIG. 8 illustrates the binding of HER2-targeting 4-1BB lipocalins to human 4-1BB (CD137) expressing reporter cell line Jurkat-hu4-1BB-NFκB-luc2. The concentration of bispecific, bivalent 2+2 anti-HER2, anti-4-1BB lipocalin huIgG1 PGLALA (termed HER2 (TRAS)×4-1BB lipocalin huIgG1 PG LALA 2+2, open down-facing triangle and dotted line) or bispecific, monovalent 1+2 anti-HER2, anti-4-1BB lipocalin huIgG1 PGLALA (termed HER2 (TRAS)×4-1BB lipocalin huIgG1 PG LALA 1+2, filled black triangle and line) or its controls is blotted against the geo mean of fluorescence intensity (gMFI) of the PE-conjugated secondary detection antibody. All values are baseline corrected by subtracting the baseline values of the blank control (e.g. no primary only secondary detection antibody).

The activation of the NFκB signaling pathway by measuring the NFκB-mediated luciferase activity in a Jurkat-hu4-1BB-NFkB-luc2 reporter cell line is shown in FIGS. 9A to 9D. To test the functionality of bispecific, bivalent 2+2 anti-HER2, anti-4-1BB lipocalin huIgG1 PG LALA (termed HER2 (TRAS)×4-1BB lipocalin huIgG1 PG LALA 2+2, open, down-facing open black triangle and dotted line) or bispecific, monovalent 1+2 anti-HER2, anti-4-1BB lipocalin huIgG1 PGLALA (termed HER2 (TRAS)×4-1BB lipocalin huIgG1 PG LALA 1+2, filled black triangle and line) or the control molecule bispecific, bivalent 2+2 anti-HER2, anti-4-1BB lipocalin huIgG4 SP (termed HER2 (TRAS)×4-1BB lipocalin huIgG4 SP 2+2, half-filled black hexamer and dotted line) or control molecules were incubated at different titrated concentrations with the reporter cell line Jurkat-hu4-1BB-NFκB-luc2 in the absence or presence of HER2-expressing cell lines NCI-N87, KPL4 or SK-Br3. All molecules failed to activate 4-1BB signaling in the absence of HER2-expressing cells, as no crosslinking occurred. In the presence of HER2-expressing cells only bispecific molecules binding HER2 and 4-1BB lead to NFκB activation on the reporter cell line. The results in the absence of HER2+ cells are shown in FIG. 9A, in the presence of HER2-expressing cell line SK-Br3 in FIG. 9B, in the presence of HER2-expressing cell line KPL4 in FIG. 9C or in the presence of HER2-expressing cell line NCI-N87 in FIG. 9D.

### DETAILED DESCRIPTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH), but it may also be provided by a scaffold antigen binding protein, in particular a lipocalin mutein.

As used herein, the term “antigen binding domain capable of specific binding to a target cell antigen” or “moiety capable of specific binding to a target cell antigen” refers to a polypeptide molecule that specifically binds to a target cell antigen. In one aspect, the antigen binding domain is able to direct the entity to which it is attached (e.g. the lipocalin mutein capable of specific binding to 4-1BB) to a target site, for example to a specific type of tumor cell bearing the target cell antigen. Antigen binding domains capable of specific binding to target cell antigen include antibodies and fragments thereof as further defined herein. In addition, moieties capable of specific binding to a target cell antigen include scaffold antigen binding proteins as further defined herein. In relation to an antibody or fragment thereof, the term “antigen binding domain capable of specific binding to a target cell antigen” comprises an antibody light chain variable region (VL) and an antibody heavy chain variable region (VH).

As used herein, the term “Fab fragment capable of specific binding to a target cell antigen” refers to a Fab molecule that specifically binds to the target cell antigen. In one aspect, the antigen binding moiety is able to activate signaling through its target cell antigen. In a particular aspect, the antigen binding moiety is able to direct the entity to which it is attached (e.g. the lipocalin mutein) to a target site, for example to a specific type of tumor cell or tumor stroma bearing the target cell antigen.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants (targets). Typically, a bispecific antigen binding molecule comprises two antigen binding sites, each of which is specific for a different antigenic determinant. In a particular aspect, a bispecific antigen binding molecule comprises three antigen binding sites, wherein two antigen binding sites are specific for a first antigenic determinant and one is specific for a second antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites in an antigen binding molecule. As such, the terms “monovalent”, “bivalent”, “tetravalent”, and “hexavalent” denote the presence of one binding site, two binding sites, four binding sites, and six binding sites, respectively, in an antigen binding molecule.

The term “monovalent to an antigen” as used within the current application denotes the presence of only one binding site for said antigen in the antigen binding molecule. The term “monovalent to a target cell antigen” as used within the current application denotes the presence of only one binding site for said target cell antigen in the antigen binding molecule.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), c (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}. In one aspect, the term “Fab fragment” also includes a cross-Fab fragment.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalins), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can beengineered to include upto 25 amino acids without affecting the overall fold of the microprotein. For further details of engineered knottin domains, see WO2008098796.

Lipocalins are a family of extracellular proteins which transport small hydrophobic molecules such as steroids, bilins, retinoids and lipids. Lipocalins are monomeric proteins of approximately 18-20 kDa in weight that exhibit a binding site with high structural plasticity, which is composed of four peptide loops mounted on a stable b-barrel scaffold (Skerra, FEBS Journal 2008, 275, 2677-2683). They have thus a rigid beta-sheet secondary structure with a number of loops at the open end of the conical structure which can be engineered to bind to different target antigens. Thereby, lipocalin muteins with specificity for a certain target antigen are produced. “Lipocalin muteins” are mutated proteins, wherein one or more amino acids are exchanged, deleted or inserted, compared to the naturally occurring (wild-type) lipocalin. The term lipocalin mutein also includes fragments or variants of the wild-type lipocalin. The lipocalin muteins as described herein are between 160-180 amino acids in size. In a particular aspect, the lipocalin mutein is a polypeptide defined by its supersecondary structure, namely cylindrical β-pleated sheet supersecondary structural region comprising eight β-strands connected pair-wise by four loops at one end to define thereby a binding pocket, wherein at least one amino acid of each of at least three of said four loops has been mutated and wherein said lipocalin is effective to bind 4-1BB with detectable affinity.

In one aspect, a lipocalin mutein disclosed herein is a mutein derived from human tear lipocalin (TLPC or Tlc), also termed tear pre-albumin or von Ebner gland protein. The term “human tear lipocalin” or “Tlc” as used herein refers to the mature human tear lipocalin with SWISS-PROT/UniProt Data Bank Accession Number P31025 (Isoform 1). A lipocalin mutein of this type is thus derived from the amino acid sequence of SEQ ID NO:90. In a particular, the lipocalin mutein disclosed herein is a mutein derived from mature human neutrophil gelatinase-associated lipocalin (huNGAL) with the SWISS-PROT/UniProt Data Bank Accession Number P80188. A lipocalin mutein of this type can be designated as “an huNGAL mutein” and is derived from a polypeptide of the amino acid sequence of SEQ ID NO:1. In some aspects, a lipocalin mutein capable of specific binding to 4-1BB with detectable affinity may include at least one amino acid substitution of a native cysteine residue by another amino acid, for example, a serine residue. In some other aspects, a lipocalin mutein capable of specific binding to 4-1BB with detectable affinity may include one or more non-native cysteine residues substituting one or more amino acids of a wild-type lipocalin. In a further particular aspect, a lipocalin mutein capable of specific binding to 4-1BB includes at least two amino acid substitutions of a native amino acid by a cysteine residue, hereby to form one or more cysteine bridges. In some embodiments, said cysteine bridge may connect at least two loop regions. In a related aspect, the disclosure teaches one or more lipocalin muteins that are capable of activating downstream signaling pathways of 4-1BB by binding to 4-1BB.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (K_{D}), which is the ratio of dissociation and association rate constants (koff and kon, respectively). Affinity can be measured by common methods known in the art, including those described herein. A particular method for measuring affinity is Surface Plasmon Resonance (SPR).

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

A “target cell antigen” as used herein refers to an antigenic determinant presented on the surface of a target cell, for example a cell in a tumor such as a cancer cell or a cell of the tumor stroma. In certain embodiments, the target cell antigen is an antigen on the surface of a tumor cell. In one embodiment, target cell antigen is selected from the group consisting of Fibroblast Activation Protein (FAP), HER2, Carcinoembryonic Antigen (CEA), Melanoma-associated Chondroitin Sulfate Proteoglycan (MCSP), Epidermal Growth Factor Receptor (EGFR), CD19, CD20 and CD33. In particular, the target cell antigen is Fibroblast Activation Protein (FAP) or HER2.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:91), or NCBI (www.ncbi.nlm.nih.gov/) RefSeq NP_004451.2. The extracellular domain (ECD) of human FAP extends from amino acid position 26 to 760. The amino acid sequence of a His-tagged human FAP ECD is shown in SEQ ID NO:92. The amino acid sequence of mouse FAP is shown in UniProt accession no. P97321 (version 126, SEQ ID NO:93), or NCBI RefSeq NP_032012.1. The extracellular domain (ECD) of mouse FAP extends from amino acid position 26 to 761. SEQ ID NO. 94 shows the amino acid sequence of a His-tagged mouse FAP ECD. SEQ ID NO:95 shows the amino acid sequence of a His-tagged cynomolgus FAP ECD. Preferably, an anti-FAP binding molecule of the invention binds to the extracellular domain of FAP. Exemplary anti-FAP binding molecules are described in International Patent Application No. WO 2012/020006 A2.

The term “capable of specific binding to FAP” refers to an antigen binding molecule that is capable of binding to FAP with sufficient affinity such that the antigen binding molecule is useful as a diagnostic and/or therapeutic agent in targeting FAP. The antigen binding molecule includes but is not limited to, antibodies, Fab molecules, crossover Fab molecules, single chain Fab molecules, Fv molecules, scFv molecules, single domain antibodies, and VH and scaffold antigen binding protein. In one aspect, the extent of binding of an anti-FAP antigen binding molecule to an unrelated, non-FAP protein is less than about 10% of the binding of the antigen binding molecule to FAP as measured, e.g., by surface plasmon resonance (SPR). In particular, an antigen binding molecule that is capable of specific binding to FAP has a dissociation constant (K_{d}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain aspects, an anti-FAP antigen binding molecule binds to FAP from different species. In particular, the anti-FAP antigen binding molecule binds to human and cynomolgus FAP or to human, cynomolgus and mouse FAP.

The term “Carcinoembroynic antigen (CEA)”, also known as Carcinoembryonic antigen-related cell adhesion molecule 5 (CEACAMS), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:96). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarström S., Semin Cancer Biol. 9(2):67-81 (1999)), making it inaccessible to antibody in the blood stream. In contrast to normal tissue, CEA tends to be expressed over the entire surface of cancerous cells (Hammarström S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “Melanoma-associated Chondroitin Sulfate Proteoglycan (MCSP)”, also known as Chondroitin Sulfate Proteoglycan 4 (CSPG4) refers to any native MCSP from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (version 103, SEQ ID NO:97). The term “Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533 (version 211, SEQ ID NO:98).

The term “CD19” refers to B-lymphocyte antigen CD19, also known as B-lymphocyte surface antigen B4 or T-cell surface antigen Leu-12 and includes any native CD19 from any vertebrate source, including mammals such as primates (e.g. humans) non-human primates (e.g. cynomolgus monkeys) and rodents (e.g. mice and rats), unless otherwise indicated. The amino acid sequence of human CD19 is shown in Uniprot accession no. P15391 (version 160, SEQ ID NO:99). The term encompasses “full-length” unprocessed human CD19 as well as any form of human CD19 that results from processing in the cell as long as the antibody as reported herein binds thereto. CD19 is a structurally distinct cell surface receptor expressed on the surface of human B cells, including, but not limited to, pre-B cells, B cells in early development {i.e., immature B cells), mature B cells through terminal differentiation into plasma cells, and malignant B cells. CD19 is expressed by most pre-B acute lymphoblastic leukemias (ALL), non-Hodgkin's lymphomas, B cell chronic lymphocytic leukemias (CLL), pro-lymphocytic leukemias, hairy cell leukemias, common acute lymphocytic leukemias, and some Null-acute lymphoblastic leukemias. The expression of CD19 on plasma cells further suggests it may be expressed on differentiated B cell tumors such as multiple myeloma. Therefore, the CD19 antigen is a target for immunotherapy in the treatment of non-Hodgkin's lymphoma, chronic lymphocytic leukemia and/or acute lymphoblastic leukemia.

“CD20” refers to B-lymphocyte antigen CD20, also known as membrane-spanning 4-domains subfamily A member 1 (MS4A1), B-lymphocyte surface antigen B1 or Leukocyte surface antigen Leu-16, and includes any native CD20 from any vertebrate source, including mammals such as primates (e.g. humans) non-human primates (e.g. cynomolgus monkeys) and rodents (e.g. mice and rats), unless otherwise indicated. The amino acid sequence of human CD20 is shown in Uniprot accession no. P11836 (version 149, SEQ ID NO:100). “CD33” refers to Myeloid cell surface antigen CD33, also known as SIGLEC3 or gp67, and includes any native CD33 from any vertebrate source, including mammals such as primates (e.g. humans) non-human primates (e.g. cynomolgus monkeys) and rodents (e.g. mice and rats), unless otherwise indicated. The amino acid sequence of human CD33 is shown in Uniprot accession no. P20138 (version 157, SEQ ID NO:101).

The term “HER2”, also known as “ErbB2”, “ErbB2 receptor”, or “c-Erb-B2”, refers to any native, mature HER2 which results from processing of a HER2 precursor protein in a cell. The term includes HER2 from any vertebrate source, including mammals such as primates (e.g. humans and cynomolgus monkeys) and rodents (e.g., mice and rats), unless otherwise indicated. The term also includes naturally occurring variants of HER2, e.g., splice variants or allelic variants. The amino acid sequence of an exemplary human HER2 protein is shown in SEQ ID NO:102.

The term “capable of specific binding to HER2” refers to an antigen binding molecule that is capable of binding to HER2 with sufficient affinity such that the antigen binding molecule is useful as a diagnostic and/or therapeutic agent in targeting HER2. The antigen binding molecule includes but is not limited to, antibodies, Fab molecules, crossover Fab molecules, single chain Fab molecules, Fab molecules, scFv molecules, single domain antibodies, and VH and scaffold antigen binding protein. In one aspect, the extent of binding of an anti-HER2 antigen binding molecule to an unrelated, non-HER2 protein is less than about 10% of the binding of the antigen binding molecule to HER2 as measured, e.g., by surface plasmon resonance (SPR). In particular, an antigen binding molecule that is capable of specific binding to HER2 has a dissociation constant (K_{d}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain aspects, an anti-HER2 antigen binding molecule binds to HER2 from different species. In particular, the anti-HER2 antigen binding molecule binds to human and cynomolgus HER2.

The term “epitope” denotes the site on an antigen, either proteinaceous or non-proteinaceous, to which an anti-[[PRO]] antibody binds. Epitopes can be formed from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

The “epitope 4D5” or “4D5 epitope” or “4D5” is the region in the extracellular domain of HER2 to which the antibody 4D5 (ATCC CRL 10463) and trastuzumab bind. This epitope is close to the transmembrane domain of HER2, and within domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 550 to about residue 610, inclusive, of human HER2 (SEQ ID NO: 102).

The “epitope 2C4” or “2C4 epitope” is the region in the extracellular domain of HER2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 2C4 epitope of HER2. Epitope 2C4 comprises residues from domain II in the extracellular domain of HER2. The 2C4 antibody and pertuzumab bind to the extracellular domain of HER2 at the junction of domains I, II and III (Franklin et al. Cancer Cell 5:317-328 (2004)).

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one aspect, for the VL, the subgroup is subgroup kappa I as in Kabat et al., supra. In one aspect, for the VH, the subgroup is subgroup III as in Kabat et al., supra.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, Md. (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, Md. (1991), NIH Publication 91-3242.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fe region” herein is used to define a C-terminal region of an antibody heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one embodiment, a heavy chain including an Fc region as specified herein, comprised in an antibody according to the invention, comprises an additional C-terminal glycine-lysine dipeptide (G446 and K447, numbering according to EU index of Kabat). In one embodiment, a heavy chain including an Fc region as specified herein, comprised in an antibody according to the invention, comprises an additional C-terminal glycine residue (G446, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described.

The term “wild-type Fe domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. Wild-type Fc-regions are denoted in SEQ ID NO: 122 (IgG1, caucasian allotype), SEQ ID NO: 123 (IgG1, afroamerican allotype), SEQ ID NO: 124 (IgG2), SEQ ID NO: 125 (IgG3) and SEQ ID NO: 126 (IgG4).

The term “variant (human) Fe domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU index of Kabat et al, Sequences of Proteins of Immunological Interest, 5th Ed. Public Health Service, National Institutes of Health, Bethesda, Md., 1991.

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The “Tumor Necrosis factor receptor superfamily” or “TNF receptor superfamily” currently consists of 27 receptors. It is a group of cytokine receptors characterized by the ability to bind tumor necrosis factors (TNFs) via an extracellular cysteine-rich domain (CRD). These pseudorepeats are defined by intrachain disulphides generated by highly conserved cysteine residues within the receptor chains. With the exception of nerve growth factor (NGF), all TNFs are homologous to the archetypal TNF-alpha. In their active form, the majority of TNF receptors form trimeric complexes in the plasma membrane. Accordingly, most TNF receptors contain transmembrane domains (TMDs). Several of these receptors also contain intracellular death domains (DDs) that recruit caspase-interacting proteins following ligand binding to initiate the extrinsic pathway of caspase activation. Other TNF superfamily receptors that lack death domains bind TNF receptor-associated factors and activate intracellular signaling pathways that can lead to proliferation or differentiation. These receptors can also initiate apoptosis, but they do so via indirect mechanisms. In addition to regulating apoptosis, several TNF superfamily receptors are involved in regulating immune cell functions such as B cell homeostasis and activation, natural killer cell activation, and T cell co-stimulation. Several others regulate cell type-specific responses such as hair follicle development and osteoclast development. Members of the TNF receptor superfamily include the following: Tumor necrosis factor receptor 1 (1A) (TNFRSF1A, CD120a), Tumor necrosis factor receptor 2 (1B) (TNFRSF1B, CD120b), Lymphotoxin beta receptor (LTBR, CD18), OX40 (TNFRSF4, CD134), CD40 (Bp50), Fas receptor (Apo-1, CD95, FAS), Decoy receptor 3 (TR6, M68, TNFRSF6B), CD27 (S152, Tp55), CD30 (Ki-1, TNFRSF8), 4-1BB (CD137, TNFRSF9), DR4 (TRAILR1, Apo-2, CD261, TNFRSF10A), DR5 (TRAILR2, CD262, TNFRSF10B), Decoy Receptor 1 (TRAILR3, CD263, TNFRSF10C), Decoy Receptor 2 (TRAILR4, CD264, TNFRSF10D), RANK (CD265, TNFRSF11A), Osteoprotegerin (OCIF, TR1, TNFRSF11B), TWEAK receptor (Fn14, CD266, TNFRSF12A), TACI (CD267, TNFRSF13B), BAFF receptor (CD268, TNFRSF13C), Herpesvirus entry mediator (HVEM, TR2, CD270, TNFRSF14), Nerve growth factor receptor (p75NTR, CD271, NGFR), B-cell maturation antigen (CD269, TNFRSF17), Glucocorticoid-induced TNFR-related (GITR, AITR, CD357, TNFRSF18), TROY (TNFRSF19), DR6 (CD358, TNFRSF21), DR3 (Apo-3, TRAMP, WS-1, TNFRSF25) and Ectodysplasin A2 receptor (XEDAR, EDA2R).

Several members of the tumor necrosis factor receptor (TNFR) family function after initial T cell activation to sustain T cell responses. The term “costimulatory TNF receptor family member” or “costimulatory TNF family receptor” refers to a subgroup of TNF receptor family members, which are able to costimulate proliferation and cytokine production of T-cells. The term refers to any native TNF family receptor from any vertebrate source, including mammals such as primates (e.g. humans), non-human primates (e.g. cynomolgus monkeys) and rodents (e.g. mice and rats), unless otherwise indicated. In specific embodiments of the invention, costimulatory TNF receptor family members are selected from the group consisting of OX40 (CD134), 4-1BB (CD137), CD27, HVEM (CD270), CD30, and GITR, all of which can have costimulatory effects on T cells. More particularly, the costimulatory TNF receptor family member is 4-1BB.

The term “4-1BB”, as used herein, refers to any native 4-1BB from any vertebrate source, including mammals such as primates (e.g. humans) and rodents (e.g., mice and rats), unless otherwise indicated. The term encompasses “full-length,” unprocessed 4-1BB as well as any form of 4-1BB that results from processing in the cell. The term also encompasses naturally occurring variants of 4-1BB, e.g., splice variants or allelic variants. The amino acid sequence of an exemplary human 4-1BB is shown in SEQ ID NO:103 (Uniprot accession no. Q07011), the amino acid sequence of an exemplary murine 4-1BB is shown in SEQ ID NO: 104 (Uniprot accession no. P20334) and the amino acid sequence of an exemplary cynomolgous 4-1BB (from Macaca mulatta) is shown in SEQ ID NO:105 (Uniprot accession no. F6W5G6).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 1 and 4, in particular 2, i.e. the peptides selected from the group consisting of GGGGS (SEQ ID NO:75), GGGGSGGGGS (SEQ ID NO:76), SGGGGSGGGG (SEQ ID NO:77), (G₄S)₃ or GGGGSGGGGSGGGGS (SEQ ID NO:78), GGGGSGGGGSGGGG or G₄(SG₄)₂ (SEQ ID NO:79), and (G₄S)₄ or GGGGSGGGGSGGGGSGGGGS (SEQ ID NO:80), but also include the sequences GSPGSSSSGS (SEQ ID NO:81), GSGSGSGS (SEQ ID NO:82), GSGSGNGS (SEQ ID NO:83), GGSGSGSG (SEQ ID NO:84), GGSGSG (SEQ ID NO:85), GGSG (SEQ ID NO:86), GGSGNGSG (SEQ ID NO:87), GGNGSGSG (SEQ ID NO:88) and GGNGSG (SEQ ID NO:89). Peptide linkers of particular interest are (G₄S)₂ or GGGGSGGGGS (SEQ ID NO:76), (G₄S)₃ (SEQ ID NO:78) and (G₄S)₄ (SEQ ID NO:80), more particularly (G₄S)₃ (SEQ ID NO:78). Further peptide linkers are selected from the group consisting of SEQ ID NO:113, SEQ ID NO:114, SEQ ID NO:115, SEQ ID NO:116; SEQ ID NO:117, SEQ ID NO:118, SEQ ID NO:119, SEQ ID NO:120 and SEQ ID NO:121.

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “fusion polypeptide” or “fusion protein” as used herein refers to a single chain polypeptide composed of an antibody fragment and a peptide that is not derived from an antibody. In one aspect, a fusion polypeptide is composed of a lipocalin mutein that is connected via a peptide bond to the Fc region of an antibody, optionally via a peptide linker. The fusion may occur by directly linking the N or C-terminal amino acid of the lipocalin mutein via a peptide linker to the C- or N-terminal amino acid of heavy chain.

By “fused” or “connected to” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www. ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more CDRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a bispecific antigen binding molecule with an N-terminal methionyl residue.

In certain aspects, the bispecific antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the bispecific antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the bispecific antigen binding molecule may be made in order to create variants with certain improved properties. In one aspect, variants of the bispecific antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the bispecific antigen binding molecules of the invention include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain aspects, it may be desirable to create cysteine engineered variants of the bispecific antigen binding molecule of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antigen binding molecules may be generated as described, e.g., in U.S. Pat. No. 7,521,541.

In certain aspects, the bispecific antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

In another aspect, immunoconjugates of the bispecific antigen binding molecules provided herein maybe obtained. An “immunoconjugate” is an antibody conjugated to one or more heterologous molecule(s), including but not limited to a cytotoxic agent.

The term “nucleic acid” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a bispecific antigen binding molecule” refers to one or more nucleic acid molecules encoding the heavy and light chains (or fragments thereof) of the bispecific antigen binding molecule, including such nucleic acid molecule(s) in a single vector or separate vectors, and such nucleic acid molecule(s) present at one or more locations in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, carcinoma, lymphoma, blastoma, sarcoma, leukemia, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colorectal cancer (CRC), pancreatic cancer, breast cancer, triple-negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, melanoma, multiple myeloma, B-cell cancer (lymphoma), chronic lymphocytic leukemia (CLL), acute lymphoblastic leukemia (ALL), hairy cell leukemia, chronic myeloblastic leukemia, including refractory versions of any of the above cancers, or a combination of one or more of the above cancers.

A “HER2-positive” cancer comprises cancer cells which have higher than normal levels of HER2. Examples of HER2-positive cancer include HER2-positive breast cancer and HER2-positive gastric cancer. Optionally, HER2-positive cancer has an immunohistochemistry (IHC) score of 2+ or 3+ and/or an in situ hybridization (ISH) amplification ratio >2.0.

The term “early stage breast cancer (EBC)” or “early breast cancer” is used herein to refer to breast cancer that has not spread beyond the breast or the axillary lymph nodes. This includes ductal carcinoma in situ and stage I, stage IIA, stage IIB, and stage IIIA breast cancers.

Reference to a tumor or cancer as a “Stage 0”, “Stage I”, “Stage II”, “Stage III”, or “Stage IV”, and various sub-stages within this classification, indicates classification of the tumor or cancer using the Overall Stage Grouping or Roman Numeral Staging methods known in the art. Although the actual stage of the cancer is dependent on the type of cancer, in general, a Stage 0 cancer is an in situ lesion, a Stage I cancer is small localized tumor, a Stage II and III cancer is a local advanced tumor which exhibits involvement of the local lymph nodes, and a Stage IV cancer represents metastatic cancer. The specific stages for each type of tumor is known to the skilled clinician.

The term “metastatic breast cancer” means the state of breast cancer where the cancer cells are transmitted from the original site to one or more sites elsewhere in the body, by the blood vessels or lymphatics, to form one or more secondary tumors in one or more organs besides the breast.

An “advanced” cancer is one which has spread outside the site or organ of origin, either by local invasion or metastasis. Accordingly, the term “advanced” cancer includes both locally advanced and metastatic disease.

A “recurrent” cancer is one which has regrown, either at the initial site or at a distant site, after a response to initial therapy, such as surgery. A “locally recurrent” cancer is cancer that returns after treatment in the same place as a previously treated cancer. An “operable” or “resectable” cancer is cancer which is confined to the primary organ and suitable for surgery (resection). A “non-resectable” or “unresectable” cancer is not able to be removed (resected) by surgery.

Bispecific Antigen Binding Molecules of the Invention

The invention provides novel bispecific antigen binding molecule capable of bivalent binding to 4-1BB and monovalent binding to a target cell antigen comprising two lipocalin muteins capable of specific binding to 4-1BB with particularly advantageous properties such as producibility, stability, binding affinity, biological activity, targeting efficiency, reduced toxicity and reduced immunicity.

The bispecific antigen binding molecules of the invention comprise two lipocalin muteins capable of specific binding to 4-1BB that are each fused to the C-terminus of one of the subunits of the Fc domain. The geometry of the bispecific antigen binding molecule and particularly the distance between the two distinct binding sites for 4-1BB and the target cell antigen are important for optimal tumor-localized activation of the costimulatory TNF receptor, i.e. 4-1BB (M. Rothe and A. Skerrra, BioDrugs 2018, 32, 233-243. It has now also been found that an impressively better activation can be obtained when there is only one antigen binding domain for the target cell antigen is present in the molecule. The lower ratio of 1:2 of tumor-target-binding to effector-cell-target-binding, e.g. the 1:2 ratio of an antigen binding domain capable of specific binding to a target cell antigen to the lipocalin muteins capable of specific binding to 4-1BB leads to a higher density of occupancy on the tumor cells, therefore a dense crosslinking of the 4-1BB agonist on the effector cells and finally to a stronger 4-1BB receptor downstream signaling.

In a first aspect, provided is a bispecific antigen binding molecule capable of bivalent binding to 4-1BB and monovalent binding to a target cell antigen, comprising

(a) an antigen binding domain capable of specific binding to a target cell antigen, in particular a Fab fragment capable of specific binding to a target cell antigen,
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

In a further aspect, provided is a bispecific antigen binding molecule capable of bivalent binding to 4-1BB and monovalent binding to a target cell antigen, comprising

(a) an antigen binding domain, in particular a Fab fragment capable of specific binding to a target cell antigen,
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain, and wherein each of the lipocalin muteins capable of specific binding to 4-1BB is derived from mature human neutrophil gelatinase-associated lipocalin (huNGAL) of SEQ ID NO:1.

In one aspect, provided is a bispecific antigen binding molecule as defined above, wherein wherein each of the lipocalin muteins capable of specific binding to 4-1BB comprise the amino acid sequence of SEQ ID NO:2 or an amino acid sequence of SEQ ID NO:2, wherein one or more of the following amino acids are mutated as following:

(a) Q at position 20 is replaced by R, or
(b) N at position 25 is replaced by Y or D, or
(c) H at position 28 is replaced by Q, or
(d) Q at position 36 is replaced by M, or
(e) I at position 40 is replaced by N, or
(f) R at position 41 is replaced by L or K, or
(g) E at position 44 is replaced by V or D, or
(h) K at position 46 is replaced by S and the amino acids at positions 47 to 49 are deleted, or
(i) I at position 49 is replaced by H, N, V or S, or
(j) M at position 52 is replaced by S or G, or
(k) K at position 59 is replaced by N, or
(l) D at position 65 is replaced by N, or
(m) M at position 68 is replaced by D, G or A, or
(n) K at position 70 is replaced by M, T, A or S, or
(o) F at position 71 is replaced by L, or
(p) D at position 72 is replaced by L, or
(q) M at position 77 is replaced by Q, H, T, R or N, or
(s) D at position 79 is replaced by I or A, or
(t) I at position 80 is replaced by N, or
(u) W at position 81 is replaced by Q, S or M, or
(v) T at position 82 is replaced by P, or
(w) F at position 83 is replaced by L, or
(y) F at position 92 is replaced by L or S, or
(z) L at position 94 is replaced by F, or
(za) K at position 96 is replaced by F, or
(zb) F at position 100 is replaced by D, or
(zc) P at position 101 is replaced by L, or
(zd) H at position 103 is replaced by P, or
(ze) S at position 106 is replaced by Y, or
(zf) F at position 122 is replaced by Y, or
(zg) F at position 125 is replaced by S, or
(zh) F at position 127 it replaced by I, or
(zi) E at position 132 is replaced by W, or
(zj) Y at position 134 is replaced by G.

In one aspect, the lipocalin muteins capable of specific binding to 4-1BB comprise an amino acid sequence of SEQ ID NO:2, wherein 4 to 10 amino acids have been mutated as defined above. In some aspects, the lipocalin mutein capable of specific binding to 4-1BB comprises one or more of the amino acid mutations:

(d) Q at position 36 is replaced by M, or
(e) I at position 40 is replaced by N, or
(f) R at position 41 is replaced by L or K, or
(i) I at position 49 is replaced by H, N, V or S, or
(j) M at position 52 is replaced by S or G, or
(m) M at position 68 is replaced by D, G or A, or
(n) K at position 70 is replaced by M, T, A or S, or
(p) D at position 72 is replaced by L, or
(q) M at position 77 is replaced by Q, H, T, R or N, or
(s) D at position 79 is replaced by I or A, or
(u) W at position 81 is replaced by Q, S or M, or
(za) K at position 96 is replaced by F, or
(zb) F at position 100 is replaced by D, or
(zd) H at position 103 is replaced by P, or
(zg) F at position 125 is replaced by S, or
(zh) F at position 127 it replaced by I, or
(zi) E at position 132 is replaced by W, or
(zj) Y at position 134 is replaced by G.

In another aspect, the lipocalin mutein capable of specific binding to 4-1BB comprises one or more of the amino acid mutations:

(a) Q at position 20 is replaced by R, or
(b) N at position 25 is replaced by Y or D, or
(g) E at position 44 is replaced by V or D, or
(k) K at position 59 is replaced by N, or
(o) F at position 71 is replaced by L, or
(t) I at position 80 is replaced by N, or
(v) T at position 82 is replaced by P, or
(y) F at position 92 is replaced by L or S, or
(zc) P at position 101 is replaced by L, or
(zf) F at position 122 is replaced by Y.

In one aspect, each of the lipocalin muteins capable of specific binding to 4-1BB comprise an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In one aspect, each of the lipocalin muteins capable of specific binding to 4-1BB comprise an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10. In a further aspect, each of the lipocalin muteins capable of specific binding to 4-1BB comprise an amino acid sequence selected from the group consisting of SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20. In one aspect, each of the lipocalin muteins capable of specific binding to 4-1BB comprise the amino acid sequence of SEQ ID NO:2. In one aspect, both lipocalin muteins comprise an identical amino acid sequence.

In a further aspect, provided is a bispecific antigen binding molecule capable of bivalent binding to 4-1BB and monovalent binding to a target cell antigen, comprising

(a) a Fab fragment capable of specific binding to a target cell antigen,
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain, and wherein each of the lipocalin muteins capable of specific binding to 4-1BB is derived from human tear lipocalin (Tlc) of SEQ ID NO:90.

In one aspect, each of the lipocalin muteins capable of specific binding to 4-1BB comprise an amino acid sequence selected from the group consisting of SEQ ID NO:106, SEQ ID NO:107, SEQ ID NO:108, SEQ ID NO:109, SEQ ID NO:110, SEQ ID NO:111 and SEQ ID NO:112.

In one aspect, the invention provides a bispecific antigen binding molecule comprising two lipocalin muteins capable of specific binding to 4-1BB, wherein one of the lipocalin muteins is fused to the C-terminus of the first subunit of the Fc domain via a peptide linker and the other is fused to the C-terminus of the second subunit of the Fc domain via a peptide linker. In one aspect, the peptide linker has an amino acid sequence selected from the group consisting of SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:113, SEQ ID NO:114, SEQ ID NO:115, SEQ ID NO:116, SEQ ID NO: 117, SEQ ID NO:118, SEQ ID NO:119, SEQ ID NO:120 and SEQ ID NO:121. In one aspect, the peptide linker has an amino acid sequence selected from the group consisting of SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88 and SEQ ID NO:89. In another aspect, the peptide linker has an amino acid sequence selected from the group consisting of SEQ ID NO:113, SEQ ID NO:114, SEQ ID NO:115, SEQ ID NO:116, SEQ ID NO: 117, SEQ ID NO:118, SEQ ID NO:119, SEQ ID NO:120 and SEQ ID NO:121. In particular, the peptide linker has the amino acid sequence of SEQ ID NO:78, i.e. (G₄S)₃.

In a further aspect, the Fc domain is an IgG, particularly an IgG1 Fc domain or an IgG4 Fc domain. More particularly, the Fc domain is an IgG1 Fc domain. In a particular aspect, the Fc domain comprises a modification promoting the association of the first and second subunit of the Fc domain.

Fc Domain Modifications Promoting Heterodimerization

In one aspect, the bispecific antigen binding molecules of the invention comprise a Fc domain composed of a first and a second subunit capable of stable association, one Fab fragment capable of specific binding to a target cell antigen that is fused to the N-terminus of the first subunit of the Fc domain, and two lipocalin muteins capable of specific binding to 4-1BB, wherein one of the lipocalin muteins is fused to the C-terminus of the first subunit of the Fc domain and the other is fused to the C-terminus of the second subunit of the Fc domain. Thus, the bispecific antigen binding molecules of the invention comprise two non-identical polypeptide chains (“heavy chains”) comprising the first and second subunit of the Fc domain, respectively, and a light chain. Recombinant co-expression of these polypeptides and subsequent dimerization leads to several possible combinations of the two non-identical heavy chains. To improve the yield and purity of the bispecific antigen binding molecules in recombinant production, it will thus be advantageous to introduce in the Fc domain of the bispecific antigen binding molecules a modification promoting the association of the desired polypeptides.

Accordingly, the Fc domain of the bispecific antigen binding molecules of the invention comprises a modification promoting the association of the first and the second subunit of the Fc domain. The site of most extensive protein-protein interaction between the two subunits of a human IgG Fc domain is in the CH3 domain of the Fc domain. Thus, said modification is particularly in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, in a particular aspect, the invention relates to a bispecific antigen binding molecule as described herein before which comprises an IgG molecule, wherein the Fc part of the first heavy chain comprises a first dimerization module and the Fc part of the second heavy chain comprises a second dimerization module allowing a heterodimerization of the two heavy chains of the IgG molecule and the first dimerization module comprises knobs and the second dimerization module comprises holes according to the knob into hole technology.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular aspect, in the CH3 domain of the first subunit of the Fc domain of the bispecific antigen binding molecules disclosed herein an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). More particularly, in the second subunit of the Fc domain additionally the threonine residue at position 366 is replaced with a serine residue (T366S) and the leucine residue at position 368 is replaced with an alanine residue (L368A). More particularly, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). The introduction of these two cysteine residues results in the formation of a disulfide bridge between the two subunits of the Fc domain. The disulfide bridge further stabilizes the dimer (Carter, J Immunol Methods 248, 7-15 (2001)).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Fc Domain Modifications Reducing Fc Receptor Binding and/or Effector Function

The Fc domain of the bispecific antigen binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the bispecific antigen binding molecule of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of a bispecific antigen binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides capable of bivalent binding to 4-1BB and monovalent binding to a target cell antigen, comprising

(a) a Fab fragment capable of specific binding to a target cell antigen,
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain, wherein the Fc domain comprises one or more amino acid substitution that reduces binding to an Fc receptor, in particular towards Fcγ receptor.

In one aspect, the Fc domain of the bispecific antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is a trimeric TNF family ligand-containing antigen binding molecule according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions. “EU numbering” refers to the numbering according to EU index of Kabat et al, Sequences of Proteins of Immunological Interest, 5th Ed. Public Health Service, National Institutes of Health, Bethesda, Md., 1991.

In one particular aspect, the Fc domain composed of a first and a second subunit capable of stable association comprises a first subunit comprising the amino acid sequence of SEQ ID NO:128 and a second subunit comprising the amino acid sequence of SEQ ID NO:129.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position 5228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antibodies of the invention is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Particular Bispecific Antigen Binding Molecules

In one aspect, the invention provides a bispecific antigen binding molecule capable of bivalent binding to 4-1BB and monovalent binding to a target cell antigen, comprising

(a) a Fab fragment capable of specific binding to Fibroblast Activation Protein (FAP),
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

In one aspect, the Fab fragment capable of specific binding to Fibroblast Activation Protein (FAP) comprises

(a) a heavy chain variable region (V_{H}FAP) comprising (i) CDR-H1 comprising the amino acid sequence of SEQ ID NO:21, (ii) CDR-H2 comprising the amino acid sequence of SEQ ID NO:22, and (iii) CDR-H3 comprising the amino acid sequence of SEQ ID NO:23, and a light chain variable region (V_{L}FAP) comprising (iv) CDR-L1 comprising the amino acid sequence of SEQ ID NO:24, (v) CDR-L2 comprising the amino acid sequence of SEQ ID NO:25, and (vi) CDR-L3 comprising the amino acid sequence of SEQ ID NO:26, or
(b) a heavy chain variable region (V_{H}FAP) comprising (i) CDR-H1 comprising the amino acid sequence of SEQ ID NO:29, (ii) CDR-H2 comprising the amino acid sequence of SEQ ID NO:30, and (iii) CDR-H3 comprising the amino acid sequence of SEQ ID NO:31, and a light chain variable region (V_{L}FAP) comprising (iv) CDR-L1 comprising the amino acid sequence of SEQ ID NO:32, (v) CDR-L2 comprising the amino acid sequence of SEQ ID NO:33, and (vi) CDR-L3 comprising the amino acid sequence of SEQ ID NO:34.

In one aspect, the Fab fragment capable of specific binding to Fibroblast Activation Protein (FAP) comprises a heavy chain variable region (V_{H}FAP) comprising (i) CDR-H1 comprising the amino acid sequence of SEQ ID NO:21, (ii) CDR-H2 comprising the amino acid sequence of SEQ ID NO:22, and (iii) CDR-H3 comprising the amino acid sequence of SEQ ID NO:23, and a light chain variable region (V_{L}FAP) comprising (iv) CDR-L1 comprising the amino acid sequence of SEQ ID NO:24, (v) CDR-L2 comprising the amino acid sequence of SEQ ID NO:25, and (vi) CDR-L3 comprising the amino acid sequence of SEQ ID NO:26.

In one aspect, provided is a Fab fragment capable of specific binding to Fibroblast Activation Protein (FAP) comprising

(a) a heavy chain variable region (V_{H}FAP) comprising an amino acid sequence that is at least about 95%, 96%, 97%, 98%, 99% or 100% identical to the amino acid sequence of SEQ ID NO:27, and a light chain variable region (V_{L}FAP) comprising an amino acid sequence that is at least about 95%, 96%, 97%, 98%, 99% or 100% identical to the amino acid sequence of SEQ ID NO:28, or
(b) a heavy chain variable region (V_{H}FAP) comprising an amino acid sequence that is at least about 95%, 96%, 97%, 98%, 99% or 100% identical to the amino acid sequence of SEQ ID NO:35, and a light chain variable region (V_{L}FAP) comprising an amino acid sequence that is at least about 95%, 96%, 97%, 98%, 99% or 100% identical to the amino acid sequence of SEQ ID NO:36.

In one aspect, provided is a Fab fragment capable of specific binding to Fibroblast Activation Protein (FAP) comprising a heavy chain variable region (V_{H}FAP) comprising an amino acid sequence of the amino acid sequence of SEQ ID NO:27, and a light chain variable region (V_{L}FAP) comprising an amino acid sequence of SEQ ID NO:28, or a heavy chain variable region (V_{H}FAP) comprising an amino acid sequence of SEQ ID NO:35, and a light chain variable region (VLFAP) comprising an amino acid sequence of SEQ ID NO:36. In one aspect, the Fab fragment capable of specific binding to Fibroblast Activation Protein (FAP) comprising a heavy chain variable region (V_{H}FAP) comprises an amino acid sequence of the amino acid sequence of SEQ ID NO:27, and a light chain variable region (V_{L}FAP) comprising an amino acid sequence of SEQ ID NO:28.

In one aspect, the bispecific antigen binding molecule provided herein comprises a first heavy chain of SEQ ID NO:37, a second heavy chain of SEQ ID NO:38 and a light chain of SEQ ID NO:39.

In another aspect, the invention provides a bispecific antigen binding molecule capable of bivalent binding to 4-1BB and monovalent binding to a target cell antigen, comprising

(a) a Fab fragment capable of specific binding to HER2,
(b) a Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

In one aspect, the Fab fragment capable of specific binding to HER2 comprises

(a) a VH domain comprising (i) CDR-H1 comprising the amino acid sequence of SEQ ID NO:40, (ii) CDR-H2 comprising the amino acid sequence of SEQ ID NO:41, and (iii) CDR-H3 comprising the amino acid sequence of SEQ ID NO:42, and a VL domain comprising (iv) CDR-L1 comprising the amino acid sequence of SEQ ID NO:43, (v) CDR-L2 comprising the amino acid sequence of SEQ ID NO:44, and (vi) CDR-L3 comprising the amino acid sequence of SEQ ID NO:45, or
(b) a VH domain comprising (i) CDR-H1 comprising the amino acid sequence of SEQ ID NO:48, (ii) CDR-H2 comprising the amino acid sequence of SEQ ID NO:49, and (iii) CDR-H3 comprising the amino acid sequence of SEQ ID NO:50, and a VL domain comprising (iv) CDR-L1 comprising the amino acid sequence of SEQ ID NO:51, (v) CDR-L2 comprising the amino acid sequence of SEQ ID NO:52, and (vi) CDR-L3 comprising the amino acid sequence of SEQ ID NO:53, or
(c) a VH domain comprising (i) CDR-H1 comprising the amino acid sequence of SEQ ID NO:56, (ii) CDR-H2 comprising the amino acid sequence of SEQ ID NO:57, and (iii) CDR-H3 comprising the amino acid sequence of SEQ ID NO:58, and a VL domain comprising (iv) CDR-L1 comprising the amino acid sequence of SEQ ID NO:59, (v) CDR-L2 comprising the amino acid sequence of SEQ ID NO:60, and (vi) CDR-L3 comprising the amino acid sequence of SEQ ID NO:61.

In one aspect, the Fab fragment capable of specific binding to HER2 comprises (a) a VH domain comprising (i) CDR-H1 comprising the amino acid sequence of SEQ ID NO:40, (ii) CDR-H2 comprising the amino acid sequence of SEQ ID NO:41, and (iii) CDR-H3 comprising the amino acid sequence of SEQ ID NO:42, and a VL domain comprising (iv) CDR-L1 comprising the amino acid sequence of SEQ ID NO:43, (v) CDR-L2 comprising the amino acid sequence of SEQ ID NO:44, and (vi) CDR-L3 comprising the amino acid sequence of SEQ ID NO:45. In one aspect, the Fab fragment capable of specific binding to HER2 comprises a VH domain comprising (i) CDR-H1 comprising the amino acid sequence of SEQ ID NO:48, (ii) CDR-H2 comprising the amino acid sequence of SEQ ID NO:49, and (iii) CDR-H3 comprising the amino acid sequence of SEQ ID NO:50, and a VL domain comprising (iv) CDR-L1 comprising the amino acid sequence of SEQ ID NO:51, (v) CDR-L2 comprising the amino acid sequence of SEQ ID NO:52, and (vi) CDR-L3 comprising the amino acid sequence of SEQ ID NO:53.

In one aspect, provided is a Fab fragment capable of specific binding to HER2 comprising

(a) a heavy chain variable region (V_{H}HER2) comprising an amino acid sequence that is at least about 95%, 96%, 97%, 98%, 99% or 100% identical to the amino acid sequence of SEQ ID NO:46, and a light chain variable region (V_{L}HER2) comprising an amino acid sequence that is at least about 95%, 96%, 97%, 98%, 99% or 100% identical to the amino acid sequence of SEQ ID NO:47, or
(b) a heavy chain variable region (V_{H}HER2) comprising an amino acid sequence that is at least about 95%, 96%, 97%, 98%, 99% or 100% identical to the amino acid sequence of SEQ ID NO:54, and a light chain variable region (V_{L}HER2) comprising an amino acid sequence that is at least about 95%, 96%, 97%, 98%, 99% or 100% identical to the amino acid sequence of SEQ ID NO:55, or
(c) a heavy chain variable region (V_{H}HER2) comprising an amino acid sequence that is at least about 95%, 96%, 97%, 98%, 99% or 100% identical to the amino acid sequence of SEQ ID NO:62, and a light chain variable region (V_{L}HER2) comprising an amino acid sequence that is at least about 95%, 96%, 97%, 98%, 99% or 100% identical to the amino acid sequence of SEQ ID NO:63.

In one aspect, provided is a Fab fragment capable of specific binding to HER2 comprising a heavy chain variable region (V_{H}HER2) comprising an amino acid sequence of SEQ ID NO:46, and a light chain variable region (V_{L}HER2) comprising an amino acid sequence of SEQ ID NO:47, or a heavy chain variable region (V_{H}HER2) comprising an amino acid sequence of SEQ ID NO:54, and a light chain variable region (V_{L}HER2) comprising an amino acid sequence of SEQ ID NO:55, or a heavy chain variable region (V_{H}HER2) comprising an amino acid sequence of SEQ ID NO:62, and a light chain variable region (V_{L}HER2) comprising an amino acid sequence of SEQ ID NO:63. In one aspect, the Fab fragment capable of specific binding to HER2 comprises a heavy chain variable region (V_{H}HER2) comprising an amino acid sequence of SEQ ID NO:46, and a light chain variable region (V_{L}HER2) comprising an amino acid sequence of SEQ ID NO:47. In one aspect, the Fab fragment capable of specific binding to HER2 comprises a heavy chain variable region (V_{H}HER2) comprising an amino acid sequence of SEQ ID NO:54, and a light chain variable region (V_{L}HER2) comprising an amino acid sequence of SEQ ID NO:55.

In one aspect, the bispecific antigen binding molecule provided herein comprises comprising a first heavy chain of SEQ ID NO:64, a second heavy chain of SEQ ID NO:65 and a light chain of SEQ ID NO:66.

Polynucleotides

The invention further provides isolated nucleic acid encoding a bispecific antigen binding molecule as described herein or a fragment thereof.

The isolated polynucleotides encoding bispecific antigen binding molecules of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated nucleic acid encodes the entire bispecific antigen binding molecule according to the invention as described herein. In particular, the isolated polynucleotide encodes a polypeptide comprised in the bispecific antigen binding molecule according to the invention as described herein.

In one aspect, the present invention is directed to isolated nucleic acid encoding a bispecific antigen binding molecule, wherein the nucleic acid molecule comprises (a) a sequence that encodes an antigen binding domain capable of specific binding to a target cell antigen, (b) a sequence that encodes a Fc domain composed of a first and a second subunit capable of stable association and (c) a sequence that encodes the lipocalin muteins capable of specific binding to 4-1BB.

In another aspect, provided is an isolated polynucleotide encoding a bispecific antigen binding molecule, wherein the polynucleotide comprises sequences that encode (a) a Fab fragment capable of specific binding to a target cell antigen, (b) a Fc domain composed of a first and a second subunit capable of stable association, and (c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

In certain aspects, the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

Bispecific antigen binding molecules of the invention may be obtained, for example, by solid-state peptide synthesis (e.g. Merrifield solid phase synthesis) or recombinant production. For recombinant production one or more polynucleotide encoding the4bispecific antigen binding molecule or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the bispecific antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the bispecific antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the bispecific antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the bispecific antigen binding molecule or polypeptide fragments thereof is desired, DNA encoding a signal sequence may be placed upstream of the nucleic acid encoding a bispecific antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a bispecific antigen binding molecule of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a bispecific antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gemgross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr− CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing a bispecific antigen binding molecule of the invention or polypeptide fragments thereof is provided, wherein the method comprises culturing a host cell comprising polynucleotides encoding the bispecific antigen binding molecule of the invention or polypeptide fragments thereof, as provided herein, under conditions suitable for expression of the bispecific antigen binding molecule of the invention or polypeptide fragments thereof, and recovering the bispecific antigen binding molecule of the invention or polypeptide fragments thereof from the host cell (or host cell culture medium).

In the bispecific antigen binding molecule of the invention, the components (at least one moiety capable of specific binding to a target cell antigen, the polypeptides comprising a subunit of the Fc domain and a lipocalin mutein) are not genetically fused to each other. The polypeptides are designed such that its components are fused to each other directly or through a linker sequence. The composition and length of the linker may be determined in accordance with methods well known in the art and may be tested for efficacy. Examples of linker sequences between different components of the antigen binding molecules of the invention are found in the sequences provided herein. Additional sequences may also be included to incorporate a cleavage site to separate the individual components of the fusion protein if desired, for example an endopeptidase recognition sequence.

In certain embodiments the antigen binding domains capable of specific binding to a target cell antigen (e.g. Fab fragments) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e.g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the antigen binding domains capable of specific binding to a target cell antigen (e.g. Fab fragments) comprised in the antigen binding molecules of the present invention are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch. 14 (Cold Spring Harbor Laboratory, Cold Spring Harbor, N.Y.).

Bispecific antigen binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the bispecific antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the bispecific antigen binding molecule or fragments thereof can be determined by any of a variety of well-known analytical methods including gel electrophoresis, high pressure liquid chromatography, and the like. For example, the bispecific antigen binding molecules expressed as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art. Biological activity may include, e.g., the ability to enhance the activation and/or proliferation of different immune cells especially T-cells. E.g. they enhance secretion of immunomodulating cytokines. Other immunomodulating cytokines which are or can be enhanced are e.g IL2, Granzyme B etc. Biological activity may also include, cynomolgus binding crossreactivity, as well as binding to different cell types. Antigen binding molecules having such biological activity in vivo and/or in vitro are also provided.

1. Affinity Assays

The affinity of the bispecific antigen binding molecule provided herein for 4-1BB (CD137) can be determined in accordance with the methods set forth in the Examples by surface plasmon resonance (SPR), using standard instrumentation such as a BIAcore instrument (GE Healthcare), and receptors or target proteins such as may be obtained by recombinant expression. Particular conditions for the determination of the affinity towards 4-1BB are also described in WO 2018/087108. The affinity of the bispecific antigen binding molecule for the target cell antigen (such as FAP or HER2) can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Examples 1.2 and 2.2. According to one aspect, K_{D} is measured by surface plasmon resonance using a BIACORE® T100 machine (GE Healthcare) at 25° C.

2. Binding Assays and Other Assays

Binding of the bispecific antigen binding molecule provided herein to the corresponding receptor expressing cells may be evaluated using cell lines expressing the particular receptor or target antigen, for example by flow cytometry (FACS). In one aspect, fresh peripheral blood mononuclear cells (PBMCs) expressing 4-1BB can be used in the binding assay. These cells are used directly after isolation (naïve PMBCs) or after stimulation (activated PMBCs). In another aspect, activated mouse splenocytes (expressing 4-1BB) can be used to demonstrate the binding of the bispecific antigen binding molecule of the invention to 4-1BB expressing cells.

In a further aspect, cell lines expressing FAP or HER2 were used to demonstrate the binding of the antigen binding molecules to this target cell antigen.

In another aspect, competition assays may be used to identify an antigen binding molecule that competes with a specific antibody or antigen binding molecule for binding to FAP, HER2 or 4-1BB, respectively. In certain aspects, such a competing antigen binding molecule binds to the same epitope (e.g., a linear or a conformational epitope) that is bound by a specific anti-FAP antibody, an anti-HER2 antibody or a specific 4-1BB antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.).

3. Activity Assays

In one aspect, assays are provided for identifying bispecific antigen binding molecules that bind to FAP or HER2 and to 4-1BB having biological activity. Biological activity may include, e.g., agonistic signalling through 4-1BB on cancer cells expressing FAP or HER2. Bispecific antigen binding molecules identified by the assays as having such biological activity in vitro are also provided.

In certain aspects, a bispecific antigen binding molecule of the invention is tested for such biological activity. Assays for detecting the biological activity of the molecules of the invention are those described in Examples 3.3 and 4.3. Furthermore, assays for detecting cell lysis (e.g. by measurement of LDH release), induced apoptosis kinetics (e.g. by measurement of Caspase 3/7 activity) or apoptosis (e.g. using the TUNEL assay) are well known in the art. In addition, the biological activity of such complexes can be assessed by evaluating their effects on survival, proliferation and lymphokine secretion of various lymphocyte subsets such as NK cells, NKT-cells or γδ T-cells or assessing their capacity to modulate phenotype and function of antigen presenting cells such as dendritic cells, monocytes/macrophages or B-cells.

Pharmaceutical Compositions, Formulations and Routes of Administration

In a further aspect, the invention provides pharmaceutical compositions comprising any of the bispecific antigen binding molecules provided herein, e.g., for use in any of the below therapeutic methods. In one embodiment, a pharmaceutical composition comprises any of the bispecific antigen binding molecules provided herein and at least one pharmaceutically acceptable excipient. In another embodiment, a pharmaceutical composition comprises any of the bispecific antigen binding molecules provided herein and at least one additional therapeutic agent, e.g., as described below.

Pharmaceutical compositions of the present invention comprise a therapeutically effective amount of one or more bispecific antigen binding molecules dissolved or dispersed in a pharmaceutically acceptable excipient. The phrases “pharmaceutical or pharmacologically acceptable” refer to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one bispecific antigen binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the bispecific antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. In addition to the compositions described previously, the bispecific antigen binding molecules may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The bispecific antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein described may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

Therapeutic Methods and Compositions

Any of the bispecific antigen binding molecules capable of bivalent binding to 4-1BB and monovalent binding to a target cell antigen provided herein may be used in therapeutic methods.

For use in therapeutic methods, bispecific antigen binding molecules of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, bispecific antigen binding molecules capable of bivalent binding to 4-1BB and monovalent binding to a target cell antigen for use as a medicament are provided. In further aspects, bispecific antigen binding molecules of the invention for use in treating a disease, in particular for use in the treatment of cancer or an infectious disease, are provided. In certain aspects, Bispecific antigen binding molecules of the invention for use in a method of treatment are provided. In one aspect, the invention provides a bispecific antigen binding molecule as described herein for use in the treatment of a disease in an individual in need thereof. In certain aspects, the invention provides a bispecific antigen binding molecule for use in a method of treating an individual having a disease comprising administering to the individual a therapeutically effective amount of the bispecific antigen binding molecule.

In certain aspects, the disease to be treated is cancer. The term “cancer” according to the invention also comprises cancer metastases. By “metastasis” is meant the spread of cancer cells from its original site to another part of the body. Tumor metastasis often occurs even after the removal of the primary tumor because tumor cells or components may remain and develop metastatic potential. In one aspect, the bispecific antigen binding molecules capable of bivalent binding to 4-1BB and monovalent binding to a target cell antigen are for use in the treatment of solid tumors. Representative examples of solid tumors include colon carcinoma, prostate cancer, breast cancer, lung cancer, skin cancer, liver cancer, bone cancer, ovary cancer, pancreas cancer, brain cancer, head and neck cancer and lymphoma. Thus, a bispecific antigen binding molecule capable of bivalent binding to 4-1BB and monovalent binding to FAP as described herein for use in the treatment of solid tumors is provided.

In certain aspects, the disease to be treated is HER2-positive cancer. Examples of HER2-positive cancers include breast cancer, ovarian cancer, gastric cancer, bladder cancer, salivary gland, endometrial cancer, pancreatic cancer and non-small-cell lung cancer (NSCLC). Thus, a bispecific antigen binding molecule capable of bivalent binding to 4-1BB and monovalent binding to HER2 as described herein for use in the treatment of these cancers is provided. The subject, patient, or “individual” in need of treatment is typically a mammal, more specifically a human.

In another aspect, provided is a bispecfic antigen binding molecule as described herein for use in the treatment of infectious diseases, in particular for the treatment of viral infections. The term “infectious disease” refers to any disease which can be transmitted from individual to individual or from organism to organism, and is caused by a microbial agent. In a further aspect, provided is a bispecific antigen binding molecule as described herein for use in the treatment of autoimmune diseases such as for example Lupus disease. In certain aspects, the infectious disease to be treated is a chronic viral infection like HIV (human immunodeficiency virus), HBV (hepatitis B virus), HCV (hepatitis C), HSV1 (herpes simplex virus type 1), CMV (cytomegalovirus), LCMV (lymphocytic chroriomeningitis virus) or EBV (Epstein-Barr virus).

In a further aspect, the invention relates to the use of a bispecific antigen binding molecule capable of bivalent binding to 4-1BB and monovalent binding to a target cell antigen in the manufacture or preparation of a medicament for the treatment of a disease in an individual in need thereof. In one aspect, the medicament is for use in a method of treating a disease comprising administering to an individual having the disease a therapeutically effective amount of the medicament. In certain aspects, the disease to be treated is a proliferative disorder, particularly cancer. Thus, in one aspect, the invention relates to the use of a bispecific binding molecule of the invention in the manufacture or preparation of a medicament for the treatment of cancer. In one aspect, provided is the use of a bispecific binding molecule of the invention in the manufacture or preparation of a medicament for the treatment of solid tumors. In one aspect, provided is the use of a bispecific binding molecule of the invention in the manufacture or preparation of a medicament for the treatment of HER2-positive cancers. Examples of HER2-positive cancers include breast cancer, ovarian cancer, gastric cancer, bladder cancer, salivary gland, endometrial cancer, pancreatic cancer and non-small-cell lung cancer (NSCLC). In certain aspect, cancers to be treated are HER2-positive breast cancer, in particular HER2-positive metastatic breast cancer. A skilled artisan may recognize that in some cases the bispecific antigen binding molecule may not provide a cure but may only provide partial benefit. In some aspects, a physiological change having some benefit is also considered therapeutically beneficial. Thus, in some aspects, an amount of the bispecific antigen binding molecule that provides a physiological change is considered an “effective amount” or a “therapeutically effective amount”.

In certain aspects, provided is the use of a bispecific binding molecule of the invention in the manufacture or preparation of a medicament for the treatment of an infectious disease. In one aspect, the infectious disease is a chronic viral infection like HIV (human immunodeficiency virus), HBV (hepatitis B virus), HCV (hepatitis C), HSV1 (herpes simplex virus type 1), CMV (cytomegalovirus), LCMV (lymphocytic chroriomeningitis virus) or EBV (Epstein-Barr virus).

In a further aspect, the invention provides a method for treating a disease in an individual, comprising administering to said individual a therapeutically effective amount of a bispecific antigen binding molecule capable of bivalent binding to 4-1BB and monovalent binding to a target cell antigen of the invention. In one aspect a composition is administered to said individual, comprising a bispecific antigen binding molecule of the invention in a pharmaceutically acceptable form. In certain aspects, the disease to be treated is a proliferative disorder. In a particular aspect, the disease is cancer. In one aspect, the disease to be treated is an infectious disease. In certain aspects, the method further comprises administering to the individual a therapeutically effective amount of at least one additional therapeutic agent, e.g. an anti-cancer agent if the disease to be treated is cancer. In certain aspects, the method comprises further administering to the individual a therapeutically effective amount of a cytotoxic agent or another immunotherapy. An “individual” according to any of the above embodiments may be a mammal, preferably a human.

For the prevention or treatment of disease, the appropriate dosage of a bispecific antigen binding molecule of the invention (when used alone or in combination with one or more other additional therapeutic agents) will depend on the type of disease to be treated, the route of administration, the body weight of the patient, the type of antigen binding molecule, the severity and course of the disease, whether the bispecific antigen binding molecule is administered for preventive or therapeutic purposes, previous or concurrent therapeutic interventions, the patient's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein. The bispecific antigen binding molecule is suitably administered to the patient at one time or over a series of treatments. Depending on the type and severity of the disease, about 1 μg/kg to 15 mg/kg (e.g. 0.1 mg/kg-10 mg/kg) of bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the bispecific antigen binding molecule would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the bispecific antigen binding molecule). An initial higher loading dose, followed by one or more lower doses may be administered. However, other dosage regimens may be useful. The progress of this therapy is easily monitored by conventional techniques and assays.

The bispecific antigen binding molecules of the invention will generally be used in an amount effective to achieve the intended purpose. For use to treat or prevent a disease condition, the bispecific antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein. For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data. Dosage amount and interval may be adjusted individually to provide plasma levels of the bispecific antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC. In cases of local administration or selective uptake, the effective local concentration of the bispecific antigen binding molecule may not be related to plasma concentration. One skilled in the art will be able to optimize therapeutically effective local dosages without undue experimentation.

A therapeutically effective dose of the bispecific antigen binding molecules described herein will generally provide therapeutic benefit without causing substantial toxicity. Toxicity and therapeutic efficacy of a bispecific antigen binding molecule can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Bispecific antigen binding molecules that exhibit large therapeutic indices are preferred. In one aspect, the bispecific antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety). The attending physician for patients treated with fusion proteins of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The bispecific antigen binding molecules capable of bivalent binding to 4-1BB and monovalent binding to a target cell antigen of the invention may be administered in combination with one or more other agents in therapy. For instance, a bispecific antigen binding molecule of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain aspects, an additional therapeutic agent is another anti-cancer agent such as a cytotoxic, chemotherapeutic or anti-angiogenic agent.

In one aspect, the bispecific antigen binding molecules capable of bivalent binding to 4-1BB and monovalent binding to a target cell antigen of the invention may be administered in combination with an agent blocking PD-L1/PD-1 interaction. In particular, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab.

Such other agents are suitably present in combination in amounts that are effective for the purpose intended. The effective amount of such other agents depends on the amount of fusion protein used, the type of disorder or treatment, and other factors discussed above. The bispecific antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific antigen binding molecule of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antigen binding molecule of the invention.

The label or package insert indicates that the composition is used for treating the condition of choice. Moreover, the article of manufacture may comprise (a) a first container with a composition contained therein, wherein the composition comprises a 4-1BBL trimer-containing antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID NO: | Description | Sequence |
| 1 | mature huNGAL | QDSTSDLIPA PPLSKVPLQQ NFQDNQFQGK |
| | | WYVVGLAGNA ILREDKDPQK MYATIYELKE |
| | | DKSYNVTSVL FRKKKCDYWI RTFVPGCQPG |
| | | EFTLGNIKSY PGLTSYLVRV VSTNYNQHAM |
| | | VFFKKVSQNR EYFKITLYGR TKELTSELKE |
| | | NFIRFSKSLG LPENHIVFPV PIDQCIDG |
| 2 | Lipocalin mutein var.13 | QDSTSDLIPA PPLSKVPLQQ NFQDNQFHGK |
| | | WYVVGQAGNI RLREDKDPIK MMATIYELKE |
| | | DKSYDVTMVK FDDKKCMYDI WTFVPGSQPG |
| | | EFTLGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 3 | Lipocalin mutein var.12 | QDSTSDLIPA PPLSKVPLQQ NFQDNQFHGK |
| | | WYVVGQAGNI KLREDKDPNK MMATIYELKE |
| | | DKSYNVTGVT FDDKKCTYAI STFVPGSQPG |
| | | EFTLGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 4 | Lipocalin mutein var.14 | QDSTSDLIPA PPLSKVPLQQ NFQDNQFHGK |
| | | WYVVGQAGNI RLREDKDPNK MMATIYELKE |
| | | DKSYDVTAVA FDDKKCTYDI WTFVPGSQPG |
| | | EFTLGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 5 | Lipocalin mutein var.15 | QDSTSDLIPA PPLSKVPLQQ NFQDNQFHGK |
| | | WYVVGQAGNI KLREDKDPNK MMATIYELKE |
| | | DKSYDVTAVA FDDKKCTYDI WTFVPGSQPG |
| | | EFTLGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 6 | Lipocalin mutein var.16 | QDSTSDLIPA PPLSKVPLQQ NFQDNQFHGK |
| | | WYVVGQAGNI KLREDSKMMA TIYELKEDKS |
| | | YDVTGVSFDD KKCTYAIMTF VPGSQPGEFT |
| | | LGKIKSFPGH TSSLVRVVST NYNQHAMVFF |
| | | KFVFQNREEF YITLYGRTKE LTSELKENFI |
| | | RFSKSLGLPE NHIVFPVPID QCIDG |
| 7 | Lipocalin mutein var.17 | QDSTSDLIPA PPLSKVPLQQ NFQDNQFHGK |
| | | WYVVGQAGNI KLREDKDPVK MMATIYELKE |
| | | DKSYDVTGVT FDDKKCRYDI STFVPGSQPG |
| | | EFTFGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 8 | Lipocalin mutein var.18 | QDSTSDLIPA PPLSKVPLQQ NFQDNQFHGK |
| | | WYVVGQAGNI RLREDKDPHK MMATIYELKE |
| | | DKSYDVTGVT FDDKKCTYAI STFVPGSQPG |
| | | EFTLGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 9 | Lipocalin mutein var.19 | QDSTSDLIPA PPLSKVPLQQ NFQDNQFHGK |
| | | WYVVGQAGNI KLREDKDPNK MMATIYELKE |
| | | DKSYDVTGVT FDDKKCTYAI STLVPGSQPG |
| | | EFTFGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 10 | Lipocalin mutein var.20 | QDSTSDLIPA PPLSKVPLQQ NFQDNQFHGK |
| | | WYVVGQAGNI RLREDKDPSK MMATIYELKE |
| | | DKSYDVTAVT FDDKKCNYAI STFVPGSQPG |
| | | EFTLGKIKSF PGHTSSLVRV VSTNYNQHAM |
| | | VFFKFVFQNR EEFYITLYGR TKELTSELKE |
| | | NFIRFSKSLG LPENHIVFPV PIDQCIDG |
| 11 | Lipocalin mutein var.47 | QDSTSDLIPA PPLSKVPLQQ NFQDNQFHGK |
| | | WYVVGMAGNN LLREDKDPHK MSATIYELKE |
| | | DKSYNVTDVM FLDKKCQYII WTFVPGSQPG |
| | | EFTLGFIKSD PGHTSYLVRV VSTNYNQHAM |
| | | VFFKSVIQNR EWFGITLYGR TKELTSELKE |
| | | NFIRFSKSLG LPENHIVFPV PIDQCIDG |
| 12 | Lipocalin mutein var.48 | QDSTSDLIPA PPLSKVPLQQ NFQDNQFQGK |
| | | WYVVGMAGNN LLREDKDPHK MSATIYELKE |
| | | DKSYNVTDVM FLDKKCQYII WTFVPGSQPG |
| | | ELTLGFIRSD LGHTSYLVRV VSTNYNQHAM |
| | | VFFKSVIQNR EWFGITLYGR TKELTSELKE |
| | | NFIRFSKSLG LPENHIVFPV PIDQCIDG |
| 13 | Lipocalin mutein var.49 | QDSTSDLIPA PPLSKVPLQQ NFQDYQFQGK |
| | | WYVVGMAGNN LLREDKDPHK MGATIYELKE |
| | | DKSYNVTDVM LLDKKCQYII QTFVPGSQPG |
| | | ESTLGFIKSD PGHTSYLVRV VSTNYNQHAM |
| | | VFFKSVIQNR EWFGITLYGR TKELTSELKE |
| | | NFIRFSKSLG LPENHIVFPV PIDQCIDG |
| 14 | Lipocalin mutein var.50 | QDSTSDLIPA PPLSKVPLQQ NFQDNQFQGK |
| | | WYVVGMAGNN LLREDKDPHK MGATIYELKE |
| | | DKSYNVTDVM FLDKKCQHII WTFVPGSQPG |
| | | ELTLGFIKSD PGHTSYLVRV VSTNYNQHAM |
| | | VFFKSVIQNR EWFGITLYGR TKELTSELKE |
| | | NFIRFSKSLG LPENHIVFPV PIDQCIDG |
| 15 | Lipocalin mutein var.51 | QDSTSDLIPA PPLSKVPLQQ NFQDDQFQGK |
| | | WYVVGMAGNN LLREDKDPHK MGATIYELKE |
| | | DKSYNVTDVM FLDKKCQYII WTFVPGSQPG |
| | | ELTLGFIKSD PGHTSYLVRV VSTNYNQHAM |
| | | VFFKSVIQNR EWFGITLYGR TKELTSELKE |
| | | NFIRFSKSLG LPENHIVFPV PIDQCIDG |
| 16 | Lipocalin mutein var.52 | QDSTSDLIPA PPLSKVPLQQ NFQDNQFQGK |
| | | WYIVGMAGNN LLREDKDPHK MGATIYELKE |
| | | DKSYNVTDVM FLDKKCQYII WTFVPGSQPG |
| | | ELTLGFIKSD PGHTSYLVRV VSTNYNQHAM |
| | | VFFKSVIQNR EWFGITLYGR TKELTSELKE |
| | | NFIRFSKSLG LPENHIVFPV PIDQCIDG |
| 17 | Lipocalin mutein var.53 | QDSTSDLIPA PPLSKVPLQR NFQDNQFQGK |
| | | WYVVGMAGNN LLRVDKDPHK MGATIYELKE |
| | | DKSYNVTDVM FLDKKCQYII WTFVPGSQPG |
| | | ELTLGFIKSD PGHTSYLVRV VSTNYNQHAM |
| | | VYFKSVIQNR EWFGITLYGR TKELTSELKE |
| | | NFIRFSKSLG LPENHIVFPV PIDQCIDG |
| 18 | Lipocalin mutein var.54 | QDSTSDLIPA PPLSKVPLQQ NFQDNQFQGK |
| | | WYVVGMAGNN LLREDKDPHK MSATIYELKE |
| | | DKSYNVTDVM FLDKKCQYIN WPFVPGSQPG |
| | | EFTLGFIKSD LGPTSYLVRV VSTNYNQHAM |
| | | VFFKSVIQNR EWFGITLYGR TKELTSELKE |
| | | NFIRFSKSLG LPENHIVFPV PIDQCIDG |
| 19 | Lipocalin mutein var.55 | QDSTSDLIPA PPLSKVPLQQ NFQDNQFQGK |
| | | WYVVGMAGNN LLREDKDPHK MGATIYELNE |
| | | DKSYNVTDVM FLDKKCQYII WTFVPGSQPG |
| | | ELTLGFIKSD PGHTSYLVRV VSTNYNQHAM |
| | | VFFKSVIQNR EWFGITLYGR TKELTSELKE |
| | | NFIRFSKSLG LPENHIVFPV PIDQCIDG |
| 20 | Lipocalin mutein var.56 | QDSTSDLIPA PPLSKVPLQQ NFQDNQFQGK |
| | | WYVVGMAGNN LLRDDKDPHK MSATIYELKE |
| | | DKSYNVTDVM LLDKKCHYII WTFVPGSQPG |
| | | ELTLGFIKSD PGHTSYLVRV VSTNYNQHAM |
| | | VFFKSVIQNR EWFGITLYGR TKELTSELKE |
| | | NFIRFSKSLG LPENHIVFPV PIDQCIDG |
| 21 | FAP(4B9) CDR-H1 | SYAMS |
| 22 | FAP(4B9) CDR-H2 | AIIGSGASTYYADSVKG |
| 23 | FAP(4B9) CDR-H3 | GWFGGFNY |
| 24 | FAP(4B9) CDR-L1 | RASQSVTSSYLA |
| 25 | FAP(4B9) CDR-L2 | VGSRRAT |
| 26 | FAP(4B9) CDR-L3 | QQGIMLPPT |
| 27 | FAP(4B9) VH | EVQLLESGGGLVQPGGSLRLSCAASGFTFSSYA |
| | | MSWVRQAPGKGLEWVSAIIGSGASTYYADSVKG |
| | | RFTISRDNSKNTLYLQMNSLRAEDTAVYYCAKG |
| | | WFGGFNYWGQGTLVTVSS |
| 28 | FAP(4B9) VL | EIVLTQSPGTLSLSPGERATLSCRASQSVTSSY |
| | | LAWYQQKPGQAPRLLINVGSRRATGIPDRFSGS |
| | | GSGTDFTLTISRLEPEDFAVYYCQQGIMLPPTF |
| | | GQGTKVEIK |
| 29 | FAP (28H1) CDR-H1 | SHAMS |
| 30 | FAP (28H1) CDR-H2 | AIWASGEQYYADSVKG |
| 31 | FAP (28H1) CDR-H3 | GWLGNFDY |
| 32 | FAP (28H1) CDR-L1 | RASQSVSRSYLA |
| 33 | FAP (28H1) CDR-L2 | GASTRAT |
| 34 | FAP (28H1) CDR-L3 | QQGQVIPPT |
| 35 | FAP(28H1) VH | EVQLLESGGGLVQPGGSLRLSCAASGFTFSSHA |
| | | MSWVRQAPGKGLEWVSAIWASGEQYYADSVKGR |
| | | FTISRDNSKNTLYLQMNSLRAEDTAVYYCAKGW |
| | | LGNFDYWGQGTLVTVSS |
| 36 | FAP(28H1) VL | EIVLTQSPGTLSLSPGERATLSCRASQSVSRSY |
| | | LAWYQQKPGQAPRLLIIGASTRATGIPDRFSGS |
| | | GSGTDFTLTISRLEPEDFAVYYCQQGQVIPPTF |
| | | GQGTKVEIK |
| 37 | Fc hole huIgG1 PGLALA-4-1BB | See Table 2 |
| | lipocalin heavy chain | |
| 38 | VH (FAP 4B9) Fc knob huIgG1 | See Table 2 |
| | PGLALA 4-1BB lipocalin heavy | |
| | chain | |
| 39 | VL (FAP 4B9) Ckappa light chain | See Table 1 |
| 40 | heavy chain CDR-H1, pertuzumab | GFTFTDYTMD |
| 41 | heavy chain CDR-H2, pertuzumab | DVNPNSGGSIYNQRFKG |
| 42 | heavy chain CDR-H3, pertuzumab | NLGPSFYFDY |
| 43 | light chain CDR-L1, pertuzumab | KASQDVSIGVA |
| 44 | light chain CDR-L2, pertuzumab | SASYRYT |
| 45 | light chain CDR-L3, pertuzumab | QQYYIYPYT |
| 46 | heavy chain variable domain VH, | EVQLVESGGGLVQPGGSLRLSCAASGFTFTDYT |
| | pertuzumab (PER) | MDWVRQAPGKGLEWVADVNPNSGGSIYNQRFKG |
| | | RFTLSVDRSKNTLYLQMNSLRAEDTAVYYCARN |
| | | LGPSFYFDYWGQGTLVTVSS |
| 47 | light chain variable domain VL, | DIQMTQSPSSLSASVGDRVTITCKASQDVSIGV |
| | pertuzumab (PER) | AWYQQKPGKAPKLLIYSASYRYTGVPSRFSGSG |
| | | SGTDFTLTISSLQPEDFATYYCQQYYIYPYTFG |
| | | QGTKVEIK |
| 48 | heavy chain CDR-H1, trastuzumab | GFNIKDTYIH |
| 49 | heavy chain CDR-H2, trastuzumab | RIYPTNGYTRYADSVKG |
| 50 | heavy chain CDR-H3, trastuzumab | WGGDGFYAMDY |
| 51 | light chain CDR-L1, trastuzumab | RASQDVNTAVA |
| 52 | light chain CDR-L2, trastuzumab | SASFLYS |
| 53 | light chain CDR-L3, trastuzumab | QQHYTTPPT |
| 54 | heavy chain variable domain VH, | EVQLVESGGGLVQPGGSLRLSCAASGFNIKDTY |
| | trastuzumab (TRAS) | IHWVRQAPGKGLEWVARIYPTNGYTRYADSVKG |
| | | RFTISADTSKNTAYLQMNSLRAEDTAVYYCSRW |
| | | GGDGFYAMDYWGQGTLVTVSS |
| 55 | light chain variable domain VL, | DIQMTQSPSSLSASVGDRVTITCRASQDVNTAV |
| | trastuzumab (TRAS) | AWYQQKPGKAPKLLIYSASFLYSGVPSRFSGSR |
| | | SGTDFTLTISSLQPEDFATYYCQQHYTTPPTFG |
| | | QGTKVEIK |
| 56 | heavy chain CDR-H1, aff | GFTFNDYTMD |
| | pertuzumab | |
| 57 | heavy chain CDR-H2, aff | DVNPNSGGSIVNRRFKG |
| | pertuzumab | |
| 58 | heavy chain CDR-H3, aff | NLGPFFYFDY |
| | pertuzumab | |
| 59 | light chain CDR-L1, aff | KASQDVSTAVA |
| | pertuzumab | |
| 60 | light chain CDR-L2, aff | SASFRYT |
| | pertuzumab | |
| 61 | light chain CDR-L3, aff | QQHYTTPPT |
| | pertuzumab | |
| 62 | heavy chain variable domain VH, | EVQLVESGGGLVQPGGSLRLSCAASGFTFNDYT |
| | aff Pertuzumab (aff-PER) | MDWVRQAPGKGLEWVADVNPNSGGSIVNRRFKG |
| | | RFTLSVDRSKNTLYLQMNSLRAEDTAVYYCARN |
| | | LGPFFYFDYWGQGTLVTVSS |
| 63 | light chain variable domain VL, | DIQMTQSPSSLSASVGDRVTITCKASQDVSTAV |
| | aff Pertuzumab (aff-PER) | AWYQQKPGKAPKLLIYSASFRYTGVPSRFSGSR |
| | | SGTDFTLTISSLQPEDFATYYCQQHYTTPPTFG |
| | | QGTKVEIK |
| 64 | Fc hole huIgG1 PGLALA-4-1BB | See Table 5 |
| | lipocalin heavy chain | |
| 65 | VH (HER2 TRAS) Fc knob huIgG1 | See Table 5 |
| | PGLALA 4-1BB lipocalin heavy | |
| | chain | |
| 66 | VL (HER2 TRAS) Ckappa light | See Table 4 |
| | chain | |
| 67 | VH (FAP 4B9)-Fc huIgG1 | See Table 1 |
| | PGLALA-4-1BB lipocalin heavy | |
| | chain | |
| 68 | VH (HER2 TRAS)-Fc huIgG1 | See Table 4 |
| | PGLALA-4-1BB lipocalin heavy | |
| | chain | |
| 69 | VH (FAP 4B9)-Fc huIgG4 SP-4- | EVQLLESGGGLVQPGGSLRLSCAASGFTFSSYA |
| | 1BB lipocalin heavy chain | MSWVRQAPGKGLEWVSAIIGSGASTYYADSVKG |
| | | RFTISRDNSKNTLYLQMNSLRAEDTAVYYCAKG |
| | | WFGGFNYWGQGTLVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEA |
| | | AGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFELYSRLTVDKSRWQEGNVESCSVMHEAL |
| | | HNHYTQKSLSLSLGKGGGGSGGGGSGGGGSQDS |
| | | TSDLIPAPPLSKVPLQQNFQDNQFHGKWYVVGQ |
| | | AGNIRLREDKDPIKMMATIYELKEDKSYDVTMV |
| | | KFDDKKCMYDIWTFVPGSQPGEFTLGKIKSFPG |
| | | HTSSLVRVVSTNYNQHAMVFFKFVFQNREEFYI |
| | | TLYGRTKELTSELKENFIRFSKSLGLPENHIVF |
| | | PVPIDQCIDG |
| 70 | VL (FAP 4B9) light chain | EIVLTQSPGTLSLSPGERATLSCRASQSVTSSY |
| | | LAWYQQKPGQAPRLLINVGSRRATGIPDRFSGS |
| | | GSGTDFTLTISRLEPEDFAVYYCQQGIMLPPTF |
| | | GQGTKVEIKRTVAAPSVFIFPPSDEQLKSGTAS |
| | | VVCLLNNFYPREAKVQWKVDNALQSGNSQESVT |
| | | EQDSKDSTYSLSSTLTLSKADYEKHKVYACEVT |
| | | HQGLSSPVTKSFNRGEC |
| 71 | VH (DP47)-Fc huIgG4 SP-4- | EVQLLESGGGLVQPGGSLRLSCAASGFTFSSYA |
| | 1BB lipocalin heavy chain | MSWVRQAPGKGLEWVSAISGSGGSTYYADSVKG |
| | | RFTISRDNSKNTLYLQMNSLRAEDTAVYYCAKG |
| | | SGFDYWGQGTLVTVSSASTKGPSVFPLAPCSRS |
| | | TSESTAALGCLVKDYFPEPVTVSWNSGALTSGV |
| | | HTFPAVLQSSGLYSLSSVVTVPSSSLGTKTYTC |
| | | NVDHKPSNTKVDKRVESKYGPPCPPCPAPEAAG |
| | | GPSVFLFPPKPKDTLMISRTPEVTCVVVDVSQE |
| | | DPEVQFNWYVDGVEVHNAKTKPREEQFNSTYRV |
| | | VSVLTVLHQDWLNGKEYKCKVSNKGLPSSIEKT |
| | | ISKAKGQPREPQVYTLPPSQEEMTKNQVSLTCL |
| | | VKGFYPSDIAVEWESNGQPENNYKTTPPVLDSD |
| | | GSFFLYSRLTVDKSRWQEGNVFSCSVMHEALHN |
| | | HYTQKSLSLSLGKGGGGSGGGGSGGGGSQDSTS |
| | | DLIPAPPLSKVPLQQNFQDNQFHGKWYVVGQAG |
| | | NIRLREDKDPIKMMATIYELKEDKSYDVTMVKF |
| | | DDKKCMYDIWTFVPGSQPGEFTLGKIKSFPGHT |
| | | SSLVRVVSTNYNQHAMVFFKFVFQNREEFYITL |
| | | YGRTKELTSELKENFIRFSKSLGLPENHIVFPV |
| | | PIDQCIDG |
| 72 | VL (DP47) light chain | EIVLTQSPGTLSLSPGERATLSCRASQSVSSSY |
| | | LAWYQQKPGQAPRLLIYGASSRATGIPDRFSGS |
| | | GSGTDFTLTISRLEPEDFAVYYCQQYGSSPLTF |
| | | GQGTKVEIKRTVAAPSVFIFPPSDEQLKSGTAS |
| | | VVCLLNNFYPREAKVQWKVDNALQSGNSQESVT |
| | | EQDSKDSTYSLSSTLTLSKADYEKHKVYACEVT |
| | | HQGLSSPVTKSFNRGEC |
| 73 | VH (HER2 TRAS)-Fc huIgG4 SP- | EVQLVESGGGLVQPGGSLRLSCAASGFNIKDTY |
| | 4-1BB lipocalin heavy chain | IHWVRQAPGKGLEWVARIYPTNGYTRYADSVKG |
| | | RFTISADTSKNTAYLQMNSLRAEDTAVYYCSRW |
| | | GGDGFYAMDYWGQGTLVTVSSASTKGPSVFPLA |
| | | PCSRSTSESTAALGCLVKDYFPEPVTVSWNSGA |
| | | LTSGVHTFPAVLQSSGLYSLSSVVTVPSSSLGT |
| | | KTYTCNVDHKPSNTKVDKRVESKYGPPCPPCPA |
| | | PEAAGGPSVFLFPPKPKDTLMISRTPEVTCVVV |
| | | DVSQEDPEVQFNWYVDGVEVHNAKTKPREEQFN |
| | | STYRVVSVLTVLHQDWLNGKEYKCKVSNKGLPS |
| | | SIEKTISKAKGQPREPQVYTLPPSQEEMTKNQV |
| | | SLTCLVKGFYPSDIAVEWESNGQPENNYKTTPP |
| | | VLDSDGSFFLYSRLTVDKSRWQEGNVFSCSVMH |
| | | EALHNHYTQKSLSLSLGKGGGGSGGGGSGGGGS |
| | | QDSTSDLIPAPPLSKVPLQQNFQDNQFHGKWYV |
| | | VGQAGNIRLREDKDPIKMMATIYELKEDKSYDV |
| | | TMVKFDDKKCMYDIWTFVPGSQPGEFTLGKIKS |
| | | FPGHTSSLVRVVSTNYNQHAMVFFKFVFQNREE |
| | | FYITLYGRTKELTSELKENFIRFSKSLGLPENH |
| | | IVFPVPIDQCIDG |
| 74 | VL (HER2 TRAS) light chain | DIQMTQSPSSLSASVGDRVTITCRASQDVNTAV |
| | | AWYQQKPGKAPKLLIYSASFLYSGVPSRFSGSR |
| | | SGTDFTLTISSLQPEDFATYYCQQHYTTPPTFG |
| | | QGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| 75 | Peptide linker G4S | GGGGS |
| 76 | Peptide linker (G4S)2 | GGGGSGGGGS |
| 77 | Peptide linker (SG4)₂ | SGGGGSGGGG |
| 78 | Peptide linker (G4S)₃ | GGGGSGGGGSGGGGS |
| 79 | Peptide linker G4(SG4)₂ | GGGGSGGGGSGGGG |
| 80 | Peptide linker (G4S)₄ | GGGGSGGGGSGGGGSGGGGS |
| 81 | Peptide linker | GSPGSSSSGS |
| 82 | Peptide linker | GSGSGSGS |
| 83 | Peptide linker | GSGSGNGS |
| 84 | Peptide linker | GGSGSGSG |
| 85 | Peptide linker | GGSGSG |
| 86 | Peptide linker | GGSG |
| 87 | Peptide linker | GGSGNGSG |
| 88 | Peptide linker | GGNGSGSG |
| 89 | Peptide linker | GGNGSG |
| 90 | human tear lipocalin (Tlc) | ASDEEIQDVS GTWYLKAMTV DREFPEMNLE |
| | | SVTPMTLTTL EGGNLEAKVT MLISGRCQEV |
| | | KAVLEKTDEP GKYTADGGKH VAYIIRSHVK |
| | | DHYIFYCEGE LHGKPVRGVK LVGRDPKNNL |
| | | EALEDFEKAA GARGLSTESI LIPRQSETCS |
| | | PG |
| 91 | Human (hu) FAP | UniProt no. Q12884 |
| 92 | hu FAP ectodomain + poly-lys- | RPSRVHNSEENTMRALTLKDILNGTFSYKTFFP |
| | tag + his₆-tag | NWISGQEYLHQSADNNIVLYNIETGQSYTILSN |
| | | RTMKSVNASNYGLSPDRQFVYLESDYSKLWRYS |
| | | YTATYYIYDLSNGEFVRGNELPRPIQYLCWSPV |
| | | GSKLAYVYQNNIYLKQRPGDPPFQITFNGRENK |
| | | IFNGIPDWVYEEEMLATKYALWWSPNGKFLAYA |
| | | EFNDTDIPVIAYSYYGDEQYPRTINIPYPKAGA |
| | | KNPVVRIFIIDTTYPAYVGPQEVPVPAMIASSD |
| | | YYFSWLTWVTDERVCLQWLKRVQNVSVLSICDF |
| | | REDWQTWDCPKTQEHIEESRTGWAGGFFVSTPV |
| | | FSYDAISYYKIFSDKDGYKHIHYIKDTVENAIQ |
| | | ITSGKWEAINIFRVTQDSLFYSSNEFEEYPGRR |
| | | NIYRISIGSYPPSKKCVTCHLRKERCQYYTASF |
| | | SDYAKYYALVCYGPGIPISTLHDGRTDQEIKIL |
| | | EENKELENALKNIQLPKEEIKKLEVDEITLWYK |
| | | MILPPQFDRSKKYPLLIQVYGGPCSQSVRSVFA |
| | | VNWISYLASKEGMVIALVDGRGTAFQGDKLLYA |
| | | VYRKLGVYEVEDQITAVRKFIEMGFIDEKRIAI |
| | | WGWSYGGYVSSLALASGTGLFKCGIAVAPVSSW |
| | | EYYASVYTERFMGLPTKDDNLEHYKNSTVMARA |
| | | EYFRNVDYLLIHGTADDNVHFQNSAQIAKALVN |
| | | AQVDFQAMWYSDQNHGLSGLSTNHLYTHMTHFL |
| | | KQCFSLSDGKKKKKKGHHHHHH |
| 93 | mouse FAP | UniProt no. P97321 |
| 94 | Murine FAP ectodomain + poly-lys- | RPSRVYKPEGNTKRALTLKDILNGTFSYKTYFP |
| | tag + his₆-tag | NWISEQEYLHQSEDDNIVFYNIETRESYIILSN |
| | | STMKSVNATDYGLSPDRQFVYLESDYSKLWRYS |
| | | YTATYYIYDLQNGEFVRGYELPRPIQYLCWSPV |
| | | GSKLAYVYQNNIYLKQRPGDPPFQITYTGRENR |
| | | IFNGIPDWVYEEEMLATKYALWWSPDGKFLAYV |
| | | EFNDSDIPIIAYSYYGDGQYPRTINIPYPKAGA |
| | | KNPVVRVFIVDTTYPHHVGPMEVPVPEMIASSD |
| | | YYFSWLTWVSSERVCLQWLKRVQNVSVLSICDF |
| | | REDWHAWECPKNQEHVEESRTGWAGGFFVSTPA |
| | | FSQDATSYYKIFSDKDGYKHIHYIKDTVENAIQ |
| | | ITSGKWEAIYIFRVTQDSLFYSSNEFEGYPGRR |
| | | NIYRISIGNSPPSKKCVTCHLRKERCQYYTASF |
| | | SYKAKYYALVCYGPGLPISTLHDGRTDQEIQVL |
| | | EENKELENSLRNIQLPKVEIKKLKDGGLTFWYK |
| | | MILPPQFDRSKKYPLLIQVYGGPCSQSVKSVFA |
| | | VNWITYLASKEGIVIALVDGRGTAFQGDKFLHA |
| | | VYRKLGVYEVEDQLTAVRKFIEMGFIDEERIAI |
| | | WGWSYGGYVSSLALASGTGLFKCGIAVAPVSSW |
| | | EYYASIYSERFMGLPTKDDNLEHYKNSTVMARA |
| | | EYFRNVDYLLIHGTADDNVHFQNSAQIAKALVN |
| | | AQVDFQAMWYSDQNHGILSGRSQNHLYTHMTHF |
| | | LKQCFSLSDGKKKKKKGHHHHHH |
| 95 | Cynomolgus FAP | RPPRVHNSEENTMRALTLKDILNGTFSYKTFFP |
| | ectodomain + poly-lys-tag + | NWISGQEYLHQSADNNIVLYNIETGQSYTILSN |
| | his₆-tag | RTMKSVNASNYGLSPDRQFVYLESDYSKLWRYS |
| | | YTATYYIYDLSNGEFVRGNELPRPIQYLCWSPV |
| | | GSKLAYVYQNNIYLKQRPGDPPFQITFNGRENK |
| | | IFNGIPDWVYEEEMLATKYALWWSPNGKFLAYA |
| | | EFNDTDIPVIAYSYYGDEQYPRTINIPYPKAGA |
| | | KNPFVRIFIIDTTYPAYVGPQEVPVPAMIASSD |
| | | YYFSWLTWVTDERVCLQWLKRVQNVSVLSICDF |
| | | REDWQTWDCPKTQEHIEESRTGWAGGFFVSTPV |
| | | FSYDAISYYKIFSDKDGYKHIHYIKDTVENAIQ |
| | | ITSGKWEAINIFRVTQDSLFYSSNEFEDYPGRR |
| | | NIYRISIGSYPPSKKCVTCHLRKERCQYYTASF |
| | | SDYAKYYALVCYGPGIPISTLHDGRTDQEIKIL |
| | | EENKELENALKNIQLPKEEIKKLEVDEITLWYK |
| | | MILPPQFDRSKKYPLLIQVYGGPCSQSVRSVFA |
| | | VNWISYLASKEGMVIALVDGRGTAFQGDKLLYA |
| | | VYRKLGVYEVEDQITAVRKFIEMGFIDEKRIAI |
| | | WGWSYGGYVSSLALASGTGLFKCGIAVAPVSSW |
| | | EYYASVYTERFMGLPTKDDNLEHYKNSTVMARA |
| | | EYFRNVDYLLIHGTADDNVHFQNSAQIAKALVN |
| | | AQVDFQAMWYSDQNHGLSGLSTNHLYTHMTHFL |
| | | KQCFSLSDGKKKKKKGHHHHHH |
| 96 | human CEA | UniProt no. P06731 |
| 97 | human MCSP | UniProt no. Q6UVK1 |
| 98 | human EGFR | UniProt no. P00533 |
| 99 | human CD19 | UniProt no. P15391 |
| 100 | human CD20 | Uniprot no. P11836 |
| 101 | human CD33 | UniProt no. P20138 |
| 102 | human HER2, UniProt Acc. No. | MELAALCRWG LLLALLPPGA ASTQVCTGTD |
| | P04626-1 | MKLRLPASPE THLDMLRHLY QGCQVVQGNL |
| | | ELTYLPTNAS LSFLQDIQEV QGYVLIAHNQ |
| | | VRQVPLQRLR IVRGTQLFED NYALAVLDNG |
| | | DPLNNTTPVT GASPGGLREL QLRSLTEILK |
| | | GGVLIQRNPQ LCYQDTILWK DIFHKNNQLA |
| | | LTLIDTNRSR ACHPCSPMCK GSRCWGESSE |
| | | DCQSLTRTVC AGGCARCKGP LPTDCCHEQC |
| | | AAGCTGPKHS DCLACLHFNH SGICELHCPA |
| | | LVTYNTDTFE SMPNPEGRYT FGASCVTACP |
| | | YNYLSTDVGS CTLVCPLHNQ EVTAEDGTQR |
| | | CEKCSKPCAR VCYGLGMEHL REVRAVTSAN |
| | | IQEFAGCKKI FGSLAFLPES FDGDPASNTA |
| | | ETLEEITGYL YISAWPDSLP DLSVFQNLQV |
| | | IRGRILHNGA YSLTLQGLGI SWLGLRSLRE |
| | | LGSGLALIHH NTHLCFVHTV PWDQLFRNPH |
| | | QALLHTANRP EDECVGEGLA CHQLCARGHC |
| | | WGPGPTQCVN CSQFLRGQEC VEECRVLQGL |
| | | PREYVNARHC LPCHPECQPQ NGSVTCFGPE |
| | | ADQCVACAHY KDPPFCVARC PSGVKPDLSY |
| | | MPIWKFPDEE GACQPCPINC THSCVDLDDK |
| | | GCPAEQRASP LTSIISAVVG ILLVVVLGVV |
| | | FGILIKRRQQ KIRKYTMRRL LQETELVEPL |
| | | TPSGAMPNQA QMRILKETEL RKVKVLGSGA |
| | | FGTVYKGIWI PDGENVKIPV AIKVLRENTS |
| | | PKANKEILDE AYVMAGVGSP YVSRLLGICL |
| | | TSTVQLVTQL MPYGCLLDHV RENRGRLGSQ |
| | | DLLNWCMQIA KGMSYLEDVR LVHRDLAARN |
| | | VLVKSPNHVK ITDFGLARLL DIDETEYHAD |
| | | GGKVPIKWMA LESILRRRFT HQSDVWSYGV |
| | | TVWELMTFGA KPYDGIPARE IPDLLEKGER |
| | | LPQPPICTID VYMIMVKCWM IDSECRPRFR |
| | | ELVSEFSRMA RDPQRFVVIQ NEDLGPASPL |
| | | DSTFYRSLLE DDDMGDLVDA EEYLVPQQGF |
| | | FCPDPAPGAG GMVHHRHRSS STRSGGGDLT |
| | | LGLEPSEEEA PRSPLAPSEG AGSDVFDGDL |
| | | GMGAAKGLQS LPTHDPSPLQ RYSEDPTVPL |
| | | PSETDGYVAP LTCSPQPEYV NQPDVRPQPP |
| | | SPREGPLPAA RPAGATLERP KTLSPGKNGV |
| | | VKDVFAFGGA VENPEYLTPQ GGAAPQPHPP |
| | | PAFSPAFDNL YYWDQDPPER GAPPSTFKGT |
| | | PTAENPEYLG LDVPV |
| 103 | Human 4-1BB | Uniprot no. Q07011 |
| 104 | Murine 4-1BB | Uniprot no. P20334 |
| 105 | Cynomolgus 4-1BB, | Uniprot no. F6W5G6 |
| 106 | Lipocalin mutein var.32 | ASDEEIQDVS GTWYLKAMTV DEGCRPWNIF |
| | | SVTPMTLTTL EGGNLEAKVT MAIDGPAQEV |
| | | KAVLEKTDEP GKYTADGGKH VAYIIRSHVK |
| | | DHYIFYSEGV CDGSPVPGVW LVGRDPKNNL |
| | | EALEDFEKAA GARGLSTESI LIPRQSETSS |
| | | PG |
| 107 | Lipocalin mutein var.33 | TSDEEIQDVS GTWYLKAMTV DEGCRPWNIF |
| | | SVTPMTLTTL EGGNLEAKVT MAIDGPAQEV |
| | | RAVLEKTDEP GKYTADGGKH DAYIIRSHVK |
| | | DHYIFYSEGV CDGSPVPGVW LVGRDPENNL |
| | | EALEDFEKTA GARGLSTESI LIPRQSETSS |
| | | PG |
| 108 | Lipocalin mutein var.34 | ASDEEIQDVS GTWYLKAMTV DEGCRPWNIF |
| | | SVTPMTLTTL EGGNLEAKVT MAIDGPAQEV |
| | | NAVLEKTDEP GKYTADGGKH VAYIIRSHVR |
| | | DHYIFYSEGV CDGSPVPGVW LVGRDPENNL |
| | | EALEDFEKTA GARGLSTESI LIPRQSETSS |
| | | PG |
| 109 | Lipocalin mutein var.35 | VSDEEIQDVS GTWYLKAMTV DEGCRPWNIF |
| | | SVTPMTLTTL EGGNLEAKVT MAIDGPAQEV |
| | | RAVLEKTDEP GKYTADGGKH VAYIIRSHVE |
| | | DHYIFYSEGV CDGSPVPGVW LVGRDPENNL |
| | | EALEDFEKTA GARGLSTESI LIPRQSETSS |
| | | PG |
| 110 | Lipocalin mutein var.36 | ASDEEIQDVS GTWYLKAMTV DEGCRPWNIF |
| | | SVTPMTLSTL EGGNLEAKVT MAIDGPAQEV |
| | | KAVLEKTDEP GKYTADGGKH VAYIIRSHVK |
| | | DHYIFYSEGV CDGSPVPGVW LVGRDPKNNL |
| | | EALEDFEKAA GARGLSTESI LIPRQIETSS |
| | | PG |
| 111 | Lipocalin mutein var.37 | ASDEEIQDVS GTWYLKAMTV DEGCRPWNIF |
| | | SVTPMTLTTL EGGNLEAEVT MAIDGPAQEV |
| | | KAVLEKADEP GKYTADGGKH VAYIIRSHVK |
| | | DHYIFYSEGV CDGSPVPGVW LVGRDPKNNL |
| | | EALEDFEKTA GARGLSTESI LIPSQIETSS |
| | | PG |
| 112 | Lipocalin mutein var.38 | TSDEEIQDVS GTWYLKAMTV DEGCRPWNIF |
| | | SVTPMTLTTL EDGNLEAKVT MAIDGPAQEV |
| | | KAVLEKADEP GKYTADGGKH VAYIIRSHVK |
| | | DHYIFYSEGV CDGSPVPGVW LVGRDPKNNL |
| | | EALEDFEKAA GARGLSTESI LIPRQIETSS |
| | | PG |
| 113 | Peptide linker | PSTPPTNSSSTIPTPS |
| 114 | Peptide linker | GGSGNSSGSGGSPV |
| 115 | Peptide linker | ASPAAPAPASPAAPAPA |
| 116 | Peptide linker | AGSGGSGGSGGSPVPSTPPTPSPSTPPTPSPSG |
| | | GSGNSSGSGGSPVPSTPPTPSPSTPPTPSPSAS |
| 117 | Peptide linker | PSTPPTPSPSTPPTPSPSGGSGNSSGSGGSPV |
| 118 | Peptide linker | AGSGGSGGSGGSPVPSTPPTNSSSTPPTPSPSP |
| | | VPSTPPTNSSSTPPTPSPSPVPSTPPTNSSSTP |
| | | PTPSPSAS |
| 119 | Peptide linker | ASPAAPAPASPAAPAPSAPAASPAAPAPASPAA |
| | | PAPSAPA |
| 120 | Peptide linker | VDDIEGRMDE |
| 121 | Peptide linker | ENLYFQGRMDE |
| 122 | IgG1, caucasian allotype | ASTKGPSVFPLAPSSKSTSGGTAALGCLVKDYF |
| | | PEPVTVSWNSGALTSGVHTFPAVLQSSGLYSLS |
| | | SVVTVPSSSLGTQTYICNVNHKPSNTKVDKKVE |
| | | PKSCDKTHTCPPCPAPELLGGPSVFLFPPKPKD |
| | | TLMISRTPEVTCVVVDVSHEDPEVKFNWYVDGV |
| | | EVHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPGK |
| 123 | IgG1, afroamerican allotype | ASTKGPSVFPLAPSSKSTSGGTAALGCLVKDYF |
| | | PEPVTVSWNSGALTSGVHTFPAVLQSSGLYSLS |
| | | SVVTVPSSSLGTQTYICNVNHKPSNTKVDKKVE |
| | | PKSCDKTHTCPPCPAPELLGGPSVFLFPPKPKD |
| | | TLMISRTPEVTCVVVDVSHEDPEVKFNWYVDGV |
| | | EVHNAKTKPREEQYNSTYRVVSVLTVLHQDWLN |
| | | GKEYKCKVSNKALPAPIEKTISKAKGQPREPQV |
| | | YTLPPSREEMTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPGK |
| 124 | IgG2 | ASTKGPSVFPLAPCSRSTSESTAALGCLVKDYF |
| | | PEPVTVSWNSGALTSGVHTFPAVLQSSGLYSLS |
| | | SVVTVPSSNFGTQTYTCNVDHKPSNTKVDKTVE |
| | | RKCCVECPPCPAPPVAGPSVFLFPPKPKDTLMI |
| | | SRTPEVTCVVVDVSHEDPEVQFNWYVDGVEVHN |
| | | AKTKPREEQFNSTERVVSVLTVVHQDWLNGKEY |
| | | KCKVSNKGLPAPIEKTISKTKGQPREPQVYTLP |
| | | PSREEMTKNQVSLTCLVKGFYPSDISVEWESNG |
| | | QPENNYKTTPPMLDSDGSFFLYSKLTVDKSRWQ |
| | | QGNVFSCSVMHEALHNHYTQKSLSLSPGK |
| 125 | IgG3 | ASTKGPSVFPLAPCSRSTSGGTAALGCLVKDYF |
| | | PEPVTVSWNSGALTSGVHTFPAVLQSSGLYSLS |
| | | SVVTVPSSSLGTQTYTCNVNHKPSNTKVDKRVE |
| | | LKTPLGDTTHTCPRCPEPKSCDTPPPCPRCPEP |
| | | KSCDTPPPCPRCPEPKSCDTPPPCPRCPAPELL |
| | | GGPSVFLEPPKPKDTLMISRTPEVTCVVVDVSH |
| | | EDPEVQFKWYVDGVEVHNAKTKPREEQYNSTFR |
| | | VVSVLTVLHQDWLNGKEYKCKVSNKALPAPIEK |
| | | TISKTKGQPREPQVYTLPPSREEMTKNQVSLTC |
| | | LVKGFYPSDIAVEWESSGQPENNYNTTPPMLDS |
| | | DGSFFLYSKLTVDKSRWQQGNIFSCSVMHEALH |
| | | NRFTQKSLSLSPGK |
| 126 | Fc huIgG4 | ASTKGPSVFPLAPCSRSTSESTAALGCLVKDYF |
| | | PEPVTVSWNSGALTSGVHTFPAVLQSSGLYSLS |
| | | SVVTVPSSSLGTKTYTCNVDHKPSNTKVDKRVE |
| | | SKYGPPCPSCPAPEFLGGPSVFLEPPKPKDTLNI |
| | | ISRTPEVTCVVVDVSQEDPEVQFNWYVDGVEVH |
| | | NAKTKPREEQFNSTYRVVSVLTVLHQDWLNGKE |
| | | YKCKVSNKGLPSSIEKTISKAKGQPREPQVYTL |
| | | PPSQEEMTKNQVSLTCLVKGFYPSDIAVEWESN |
| | | GQPENNYKTTPPVLDSDGSFFLYSRLTVDKSRW |
| | | QEGNVFSCSVMHEALHNHYTQKSLSLSLGK |
| 127 | Fc huIgG4 SP | ASTKGPSVFPLAPCSRSTSESTAALGCLVKDYF |
| | | PEPVTVSWNSGALTSGVHTFPAVLQSSGLYSLS |
| | | SVVTVPSSSLGTKTYTCNVDHKPSNTKVDKRVE |
| | | SKYGPPCPPCPAPEAAGGPSVFLEPPKPKDTLNI |
| | | ISRTPEVTCVVVDVSQEDPEVQFNWYVDGVEVH |
| | | NAKTKPREEQFNSTYRVVSVLTVLHQDWLNGKE |
| | | YKCKVSNKGLPSSIEKTISKAKGQPREPQVYTL |
| | | PPSQEEMTKNQVSLTCLVKGFYPSDIAVEWESN |
| | | GQPENNYKTTPPVLDSDGSFFLYSRLTVDKSRW |
| | | QEGNVFSCSVMHEALHNHYTQKSLSLSLGK |
| 128 | Fc hole hu IgG1 | DKTHTCPPCPAPEAAGGPSVFLFPPKPKDTLMI |
| | | SRTPEVTCVVVDVSHEDPEVKFNWYVDGVEVHN |
| | | AKTKPREEQYNSTYRVVSVLTVLHQDWLNGKEY |
| | | KCKVSNKALGAPIEKTISKAKGQPREPQVCTLP |
| | | PSRDELTKNQVSLSCAVKGFYPSDIAVEWESNG |
| | | QPENNYKTTPPVLDSDGSFFLVSKLTVDKSRWQ |
| | | QGNVFSCSVMHEALHNHYTQKSLSLSPG |
| 129 | Fc knob hu IgG1 | DKTHTCPPCPAPEAAGGPSVFLFPPKPKDTLMI |
| | | SRTPEVTCVVVDVSHEDPEVKFNWYVDGVEVHN |
| | | AKTKPREEQYNSTYRVVSVLTVLHQDWLNGKEY |
| | | KCKVSNKALGAPIEKTISKAKGQPREPQVYTLP |
| | | PCRDELTKNQVSLWCLVKGFYPSDIAVEWESNG |
| | | QPENNYKTTPPVLDSDGSFFLYSKLTVDKSRWQ |
| | | QGNVFSCSVMHEALHNHYTQKSLSLSPG |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the EU numbering systems according to Kabat (Kabat, E. A., et al., Sequences of Proteins of Immunological Interest, 5th ed., Public Health Service, National Institutes of Health, Bethesda, Md. (1991)) as defined above.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

### Example 1

### Preparation, Purification and Characterization of Bispecific Antibodies with a Bivalent Binding to 4-1BB and Monovalent/Bivalent Binding to FAP

1.1 Generation of Bispecific Antibodies with a Bivalent Binding to 4-1BB and Monovalent or Bivalent Binding to FAP

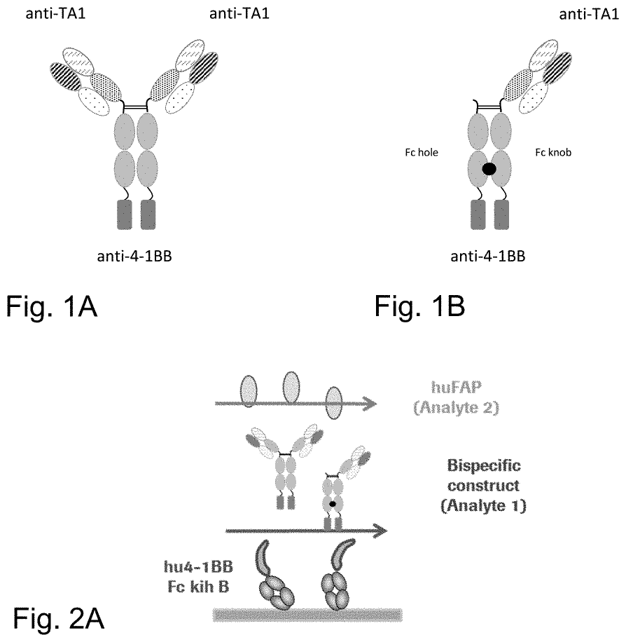

Bispecific agonistic 4-1BB antibodies with bivalent binding for 4-1BB and monovalent or bivalent to FAP, were prepared as described in FIGS. 1A and 1B. The FAP binder (clone 4B9, generation and preparation as described in WO 2012/020006 A2, which is incorporated herein by reference) and the 4-1BB binder (anticalin, generation and preparation as described in WO 2016/177802) were used to prepare the molecules described in FIGS. 1A and 1B, with TA1 being FAP. The Pro329Gly, Leu234Ala and Leu235Ala mutations were introduced in the Fc constant region of the heavy chains to abrogate binding to Fc gamma receptors according to the method described in International Patent Appl. Publ. No. WO2012/130831A1.

The variable region of heavy and light chain DNA sequences encoding the FAP(4B9) binder were subcloned in frame with either the constant heavy chain of the hole or the constant light chain of human IgG1.

The construct with bivalent binding to FAP was cloned as follows: two heavy chains comprising each VH (FAP)-Fc (hu IgG1)-(G4S)3 connector-4-1BB binding lipocalin and two light chains comprising VL(FAP)-Ckappa. The amino acid sequences for bispecific, bivalent 2+2 anti-FAP, anti-4-1BB huIgG1 PGLALA can be found in Table 1.

The construct with monovalent binding to FAP was cloned as follows: one heavy chain comprising VH (FAP)-Fc knob (hu IgG1)-(G4S)3 connector-4-1BB binding lipocalin, one heavy chain Fc hole (hu IgG1)-(G4S)3 connector-4-1BB binding lipocalin and one light chain comprising VL(FAP)-Ckappa. Combination of the Fc knob heavy chain containing the S354C/T366W mutations and the Fc hole heavy chain containing the Y349C/T366S/L368A/Y407V mutations and the anti-FAP light chain allowed the generation of a heterodimer, which includes two 4-1BB binding lipocalins. The amino acid sequences for bispecific, monovalent 2+1 anti-FAP, anti-4-1BB huIgG1 PGLALA can be found in Table 2.

TABLE 1
Amino acid sequences of mature bispecific, bivalent 2 + 2 anti-FAP,
anti-4-1BB lipocalin huIgG1 PGLALA antigen binding molecule

| SEQ ID | | |
|---|---|---|
| NO: | Description | Sequences |
| 67 | VH (FAP 4B9)- | EVQLLESGGGLVQPGGSLRLSCAASGFTFSSYAMSWVRQAPGKGLEWV |
| | Fc huIgG1-4- | SAIIGSGASTYYADSVKGRFTISRDNSKNTLYLQMNSLRAEDTAVYYC |
| | 1BB lipocalin | AKGWFGGFNYWGQGTLVTVSSASTKGPSVFPLAPSSKSTSGGTAALGC |
| | heavy chain | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALGAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGGGGGSGGGGSGGGGSQDSTSDLIPAPPLSKVPLQ |
| | | QNFQDNQFHGKWYVVGQAGNIRLREDKDPIKMMATIYELKEDKSYDVT |
| | | MVKFDDKKCMYDIWTFVPGSQPGEFTLGKIKSFPGHTSSLVRVVSTNY |
| | | NQHAMVFFKFVFQNREEFYITLYGRTKELTSELKENFIRFSKSLGLPE |
| | | NHIVFPVPIDQCIDG |
| 39 | VL (FAP 4B9)- | EIVLTQSPGTLSLSPGERATLSCRASQSVTSSYLAWYQQKPGQAPRLL |
| | Ckappa light | INVGSRRATGIPDRFSGSGSGTDFTLTISRLEPEDFAVYYCQQGIMLP |
| | Chain | PTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEKHKV |
| | | YACEVTHQGLSSPVTKSFNRGEC |

TABLE 2
Amino acid sequences of mature bispecific, monovalent 1 + 2 anti-FAP,
anti-4-1BB lipocalin huIgG1 PGLALA antigen binding molecule

| SEQ ID | | |
|---|---|---|
| NO: | Description | Sequences |
| 37 | Fc hole huIgG1- | DKTHTCPPCPAPEAAGGPSVFLFPPKPKDTLMISRTPEVTCVVVDVSH |
| | 4-1BB lipocalin | EDPEVKFNWYVDGVEVHNAKTKPREEQYNSTYRVVSVLTVLHQDWLNG |
| | heavy chain | KEYKCKVSNKALGAPIEKTISKAKGQPREPQVCTLPPSRDELTKNQVS |
| | | LSCAVKGFYPSDIAVEWESNGQPENNYKTTPPVLDSDGSFFLVSKLTV |
| | | DKSRWQQGNVFSCSVMHEALHNHYTQKSLSLSPGGGGGSGGGGSGGGG |
| | | SQDSTSDLIPAPPLSKVPLQQNFQDNQFHGKWYVVGQAGNIRLREDKD |
| | | PIKMMATIYELKEDKSYDVTMVKFDDKKCMYDIWTFVPGSQPGEFTLG |
| | | KIKSFPGHTSSLVRVVSTNYNQHAMVFFKFVFQNREEFYITLYGRTKE |
| | | LTSELKENFIRFSKSLGLPENHIVFPVPIDQCIDG |
| 38 | VH (FAP 4B9) Fc | EVQLLESGGGLVQPGGSLRLSCAASGFTFSSYAMSWVRQAPGKGLEWV |
| | knob huIgG1 4- | SAIIGSGASTYYADSVKGRFTISRDNSKNTLYLQMNSLRAEDTAVYYC |
| | 1BB lipocalin | AKGWFGGFNYWGQGTLVTVSSASTKGPSVFPLAPSSKSTSGGTAALGC |
| | heavy chain | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPEAAGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALGAPIEKTI |
| | | SKAKGQPREPQVYTLPPCRDELTKNQVSLWCLVKGFYPSDIAVEWESN |
| | | GQPENNYKTTPPVLDSDGSFELYSKLTVDKSRWQQGNVESCSVMHEAL |
| | | HNHYTQKSLSLSPGGGGGSGGGGSGGGGSQDSTSDLIPAPPLSKVPLQ |
| | | QNFQDNQFHGKWYVVGQAGNIRLREDKDPIKMMATIYELKEDKSYDVT |
| | | MVKFDDKKCMYDIWTFVPGSQPGEFTLGKIKSFPGHTSSLVRVVSTNY |
| | | NQHAMVFFKFVFQNREEFYITLYGRTKELTSELKENFIRFSKSLGLPE |
| | | NHIVFPVPIDQCIDG |
| 39 | VL (FAP 4B9) | See Table 1 |
| | Ckappa light | |
| | chain | |

The bispecific antibodies were generated by transient transfection of HEK293 EBNA cells. Cells were centrifuged and medium replaced by pre-warmed CD CHO medium. Expression vectors were mixed in CD CHO medium, PEI was added, the solution vortexed and incubated for 10 minutes at room temperature. Afterwards, cells were mixed with the DNA/PEI solution, transferred to shake flask and incubated for 3 hours at 37° C. in an incubator with a 5% CO₂ atmosphere. After the incubation, Excell medium with supplements was added. One day after transfection 12% Feed was added. Cell supernatants were harvested after 7 days and purified by standard methods. The cells were transfected with the corresponding expression vectors in a 1:1 or 1:1:1 ratio for respectively a) and b) constructs.

Proteins were purified from filtered cell culture supernatants referring to standard protocols. In brief, Fc containing proteins were purified from cell culture supernatants by affinity chromatography using Protein A. Elution was achieved at pH 3.0 followed by immediate neutralization of the sample. The protein was concentrated and aggregated protein was separated from monomeric protein by size exclusion chromatography in 20 mM histidine, 140 mM sodium chloride, pH 6.0.

The protein concentration of purified constructs was determined by measuring the optical density (OD) at 280 nm, using the molar extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII. Determination of the aggregate content was performed by HPLC chromatography using analytical size-exclusion column (TSKgel G3000 SW XL) equilibrated in a 25 mM K₂HPO₄, 125 mM NaCl, 200 mM L-Arginine Monohydrochloride, pH 6.7 running buffer at 25° C.

Table 3 summarizes the yield and final monomer content of the bispecific FAP (4B9) targeted 4-1BB binding antigen binding molecules.

TABLE 3
Biochemical analysis of bispecific 4-1BB binding
antigen binding molecules

| | Monomer | | |
|---|---|---|---|
| | [%] | Yield | CE-SDS |
| Molecule | (SEC) | [mg/l] | (non-red) |
| 2 + 2 FAP(4B9) x 4-1BB lipocalin | 99 | 250 | 88 |
| huIgG1 PGLALA | | | |
| 1 + 2 FAP(4B9) x 4-1BB lipocalin | 100 | 77 | 100 |
| huIgG1 PGLALA | | | |

For comparison, a 2+2 FAP(4B9)×4-1BB lipocalin huIgG4 SP molecule comprising the amino acid sequences of SEQ ID NO:69 and SEQ ID NO:70 and an untargeted 2+2 DP47×4-1BB lipocalin huIgG4 SP control molecule comprising the amino acid sequences of SEQ ID NO:71 and SEQ ID NO:72 were also produced.

1.2 Functional Characterization of Bispecific and Trispecific Antibodies with a Bivalent Binding to 4-1BB and Monovalent or Bivalent Binding to FAP by Surface Plasmon Resonance

The capacity of binding simultaneously human 4-1BB Fc(kih) and human FAP was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Biotinylated human 4-1BB Fc(kih) was directly coupled to a flow cell of a streptavidin (SA) sensor chip. Immobilization levels up to 500 resonance units (RU) were used.

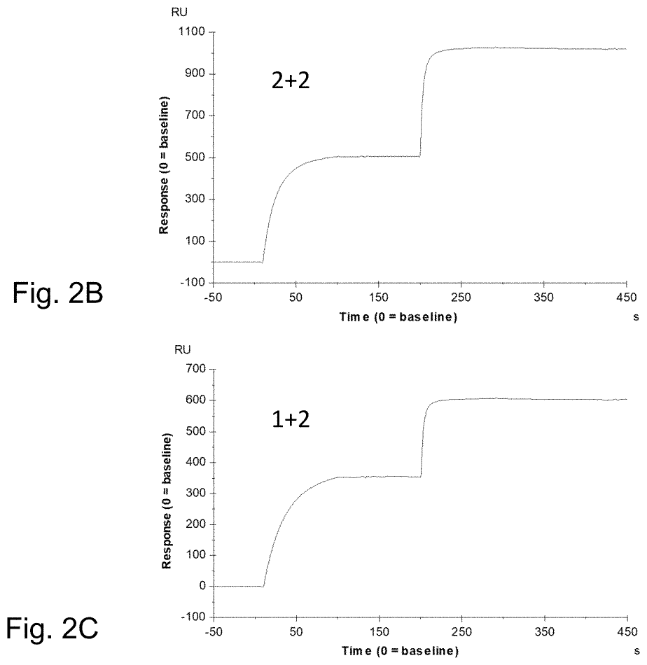

The bispecific FAP-targeted anti-4-1BB lipocalins were passed at a concentration range of 200 nM with a flow of 30 μL/minute through the flow cells over 90 seconds and dissociation was set to zero sec. Human FAP was injected as second analyte with a flow of 30 μL/minute through the flow cells over 90 seconds at a concentration of 500 nM (FIG. 2A). The dissociation was monitored for 120 sec. Bulk refractive index differences were corrected for by subtracting the response obtained in a reference flow cell, where no protein was immobilized.

As can be seen in the graphs of FIGS. 2B and 2C, both bispecific FAP targeted anti-4-1BB lipocalins could bind simultaneously human 4-1BB and human FAP.

### Example 2

### Preparation, Purification and Characterization of Bispecific Antibodies with a Bivalent Binding to 4-1BB and Monovalent/Bivalent Binding to HER2

2.1 Generation of Bispecific Antibodies with a Bivalent Binding to 4-1BB and Monovalent or Bivalent Binding to HER2

Bispecific agonistic 4-1BB antibodies with bivalent binding for 4-1BB and monovalent or bivalent binding to HER2, were prepared as described in FIGS. 1A and 1B. The HER2 binder (corresponding to trastuzumab) and the 4-1BB binder (lipocalin, generation and preparation as described in WO 2016/177802) were used to prepare the molecules described in FIGS. 1A and 1B, with TA1 being HER2. The Pro329Gly, Leu234Ala and Leu235Ala mutations were introduced in the Fc constant region of the heavy chains to abrogate binding to Fc gamma receptors according to the method described in International Patent Appl. Publ. No. WO2012/130831A1.

The variable region of heavy and light chain DNA sequences encoding the FAP(4B9) binder were subcloned in frame with either the constant heavy chain of the hole or the constant light chain of human IgG1.

The construct with bivalent binding to FAP was cloned as follows: two heavy chains comprising each VH (HER2)-Fc (hu IgG1)-(G4S)3 connector-4-1BB binding lipocalin and two light chains comprising VL(HER2)-Ckappa. The amino acid sequences for bispecific, bivalent 2+2 anti-HER2, anti-4-1BB huIgG1 PGLALA can be found in Table 4.

The construct with monovalent binding to FAP was cloned as follows: one heavy chain comprising VH (HER2)-Fc knob (hu IgG1)-(G4S)3 connector-4-1BB binding lipocalin, one heavy chain Fc hole (hu IgG1)-(G4S)3 connector-4-1BB binding lipocalin and one light chain comprising VL(HER2)-Ckappa. Combination of the Fc knob heavy chain containing the S354C/T366W mutations and the Fc hole heavy chain containing the Y349C/T366S/L368A/Y407V mutations and the anti-HER2 light chain allowed the generation of a heterodimer, which includes two 4-1BB binding lipocalins. The amino acid sequences for bispecific, monovalent 2+1 anti-HER2, anti-4-1BB huIgG1 PGLALA can be found in Table 5.

TABLE 4
Amino acid sequences of mature bispecific, bivalent 2 + 2 anti-HER2,
anti-4-1BB lipocalin huIgG1 PGLALA antigen binding molecule

| SEQ ID | | |
|---|---|---|
| NO: | Description | Sequences |
| 68 | VH (HER2)-Fc | EVQLVESGGGLVQPGGSLRLSCAASGFNIKDTYIHWVRQAPGKGLEWV |
| | huIgG1-4-1BB | ARIYPTNGYTRYADSVKGRFTISADTSKNTAYLQMNSLRAEDTAVYYC |
| | lipocalin | SRWGGDGFYAMDYWGQGTLVTVSSASTKGPSVFPLAPSSKSTSGGTAA |
| | heavy chain | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAGG |
| | | PSVFLEPPKPKDTLMISRTPEVTCVVVDVSHEDPEVKFNWYVDGVEVH |
| | | NAKTKPREEQYNSTYRVVSVLTVLHQDWLNGKEYKCKVSNKALGAPIE |
| | | KTISKAKGQPREPQVYTLPPSRDELTKNQVSLTCLVKGFYPSDIAVEW |
| | | ESNGQPENNYKTTPPVLDSDGSFELYSKLTVDKSRWQQGNVESCSVMH |
| | | EALHNHYTQKSLSLSPGGGGGSGGGGSGGGGSQDSTSDLIPAPPLSKV |
| | | PLQQNFQDNQFHGKWYVVGQAGNIRLREDKDPIKMMATIYELKEDKSY |
| | | DVTMVKFDDKKCMYDIWTFVPGSQPGEFTLGKIKSFPGHTSSLVRVVS |
| | | TNYNQHAMVFFKFVFQNREEFYITLYGRTKELTSELKENFIRFSKSLG |
| | | LPENHIVFPVPIDQCIDG |
| 66 | VL (HER2)- | DIQMTQSPSSLSASVGDRVTITCRASQDVNTAVAWYQQKPGKAPKLLI |
| | Ckappa light | YSASFLYSGVPSRFSGSRSGTDFTLTISSLQPEDFATYYCQQHYTTPP |
| | chain | TEGQGTKVEIKRTVAAPSVFIFPPSDEQLKSGTASVVCLLNNEYPREA |
| | | KVQWKVDNALQSGNSQESVTEQDSKDSTYSLSSTLTLSKADYEKHKVY |
| | | ACEVTHQGLSSPVTKSFNRGEC |

TABLE 5
Amino acid sequences of mature bispecific, monovalent 1 + 2 anti-HER2,
anti-4-1BB lipocalin huIgG1 PGLALA antigen binding molecule

| SEQ ID | | |
|---|---|---|
| NO: | Description | Sequences |
| 64 | Fc hole huIgG1- | DKTHTCPPCPAPEAAGGPSVFLFPPKPKDTLMISRTPEVTCVVVDVSH |
| | 4-1BB lipocalin | EDPEVKFNWYVDGVEVHNAKTKPREEQYNSTYRVVSVLTVLHQDWLNG |
| | heavy chain | KEYKCKVSNKALGAPIEKTISKAKGQPREPQVCTLPPSRDELTKNQVS |
| | | LSCAVKGFYPSDIAVEWESNGQPENNYKTTPPVLDSDGSFFLVSKLTV |
| | | DKSRWQQGNVFSCSVMHEALHNHYTQKSLSLSPGGGGGSGGGGSGGGG |
| | | SQDSTSDLIPAPPLSKVPLQQNFQDNQFHGKWYVVGQAGNIRLREDKD |
| | | PIKMMATIYELKEDKSYDVTMVKFDDKKCMYDIWTFVPGSQPGEFTLG |
| | | KIKSFPGHTSSLVRVVSTNYNQHAMVFFKFVFQNREEFYITLYGRTKE |
| | | LTSELKENFIRFSKSLGLPENHIVFPVPIDQCIDG |
| 65 | VH (HER2) Fc | EVQLVESGGGLVQPGGSLRLSCAASGFNIKDTYIHWVRQAPGKGLEWV |
| | knob huIgG1 4- | ARIYPTNGYTRYADSVKGRFTISADTSKNTAYLQMNSLRAEDTAVYYC |
| | 1BB lipocalin | SRWGGDGFYAMDYWGQGTLVTVSSASTKGPSVFPLAPSSKSTSGGTAA |
| | heavy chain | LGCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEAAGG |
| | | PSVFLEPPKPKDTLMISRTPEVTCVVVDVSHEDPEVKFNWYVDGVEVH |
| | | NAKTKPREEQYNSTYRVVSVLTVLHQDWLNGKEYKCKVSNKALGAPIE |
| | | KTISKAKGQPREPQVYTLPPCRDELTKNQVSLWCLVKGFYPSDIAVEW |
| | | ESNGQPENNYKTTPPVLDSDGSFELYSKLTVDKSRWQQGNVESCSVMH |
| | | EALHNHYTQKSLSLSPGGGGGSGGGGSGGGGSQDSTSDLIPAPPLSKV |
| | | PLQQNFQDNQFHGKWYVVGQAGNIRLREDKDPIKMMATIYELKEDKSY |
| | | DVTMVKFDDKKCMYDIWTFVPGSQPGEFTLGKIKSFPGHTSSLVRVVS |
| | | TNYNQHAMVFFKFVFQNREEFYITLYGRTKELTSELKENFIRFSKSLG |
| | | LPENHIVFPVPIDQCIDG |
| 66 | VL (HER2) | See Table 4 |
| | Ckappa light | |
| | chain | |

The bispecific antibodies were produced and purified as described in Example 1.

Table 6 summarizes the yield and final monomer content of the bispecific HER2-targeted 4-1BB binding antigen binding molecules.

TABLE 6
Biochemical analysis of bispecific 4-1BB binding antigen
binding molecules

| | Monomer | | |
|---|---|---|---|
| | [%] | Yield | CE-SDS |
| Molecule | (SEC) | [mg/l] | (non-red) |
| 2 + 2 HER2 x 4-1BB lipocalin hulgG1 | 100 | 19 | 100 |
| PGLALA | | | |
| 1 + 2 HER2 x 4-1BB lipocalin hulgG1 | 98 | 19 | 100 |
| PGLALA | | | |

For comparison, the previously described fusion polypeptide 2+2 HER2 (TRAS)-anticalin-4-1BB human IgG4 SP comprising the amino acid sequences of SEQ ID NO:73 and SEQ ID NO:74 was also made (WO2016/177802).

2.2 Functional Characterization of Bispecific and Trispecific Antibodies with a Bivalent Binding to 4-1BB and Monovalent or Bivalent Binding to HER2 by Surface Plasmon Resonance

The capacity of binding simultaneously human 4-1BB Fc(kih) and human HER2 was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Biotinylated human 4-1BB Fc(kih) was directly coupled to a flow cell of a streptavidin (SA) sensor chip. Immobilization levels up to 500 resonance units (RU) were used.

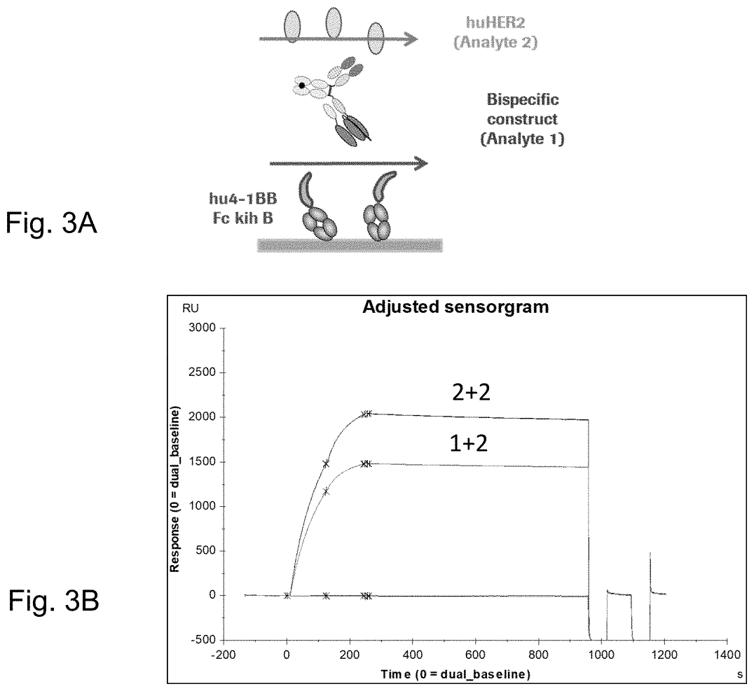

The bispecific HER2-targeted anti-4-1BB lipocalins were passed at a concentration range of 200 nM with a flow of 30 μL/minute through the flow cells over 90 seconds and dissociation was set to zero sec. Human FAP was injected as second analyte with a flow of 30 μL/minute through the flow cells over 90 seconds at a concentration of 500 nM (FIG. 3A). The dissociation was monitored for 120 sec. Bulk refractive index differences were corrected for by subtracting the response obtained in a reference flow cell, where no protein was immobilized.

As can be seen in the graphs of FIG. 3B, both bispecific HER2-targeted anti-4-1BB lipocalins could bind simultaneously human 4-1BB and human HER2.

### Example 3

### Functional Characterization of the FAP-Targeted 4-1BB Lipocalin Antigen Binding Molecules

### 3.1 Binding to Human FAP-Expressing Cell Lines

For binding to cell-surface-expressed human Fibroblast Activation Protein (FAP) NIH/3T3-huFAP clone 19 cells were used. NIH/3T3-huFAP clone 19 was generated by transfection of mouse embryonic fibroblast NIH/3T3 cells (ATCC CRL-1658) with the expression pETR4921 plasmid encoding human FAP under a CMV promoter. Cells were maintained in DMEM (GIBCO by life technologies, Cat.-No.: 42340-025) supplied with fetal bovine serum (FBS, GIBCO by Life Technologies, Cat.-No. 16000-044, Lot 941273, gamma irradiated mycoplasma free, heat inactivated), 2 mM L-alanyl-L-glutamine dipeptide (Glutqa-MAX-I, GIBCO by Life Technologies, Cat.-No. 35050-038) and 1.5 μg/mL puromycin (InvivoGen, Cat.-No.: ant-pr-5). For the binding assay, 2×10⁵ of NIH/3T3-huFAP clone 19 cells were added to each well of a round-bottom suspension cell 96-well plates (Greiner bio-one, cellstar, Cat.-No. 650185). Cells were washed once with 200 μL, DPBS and pellets were resuspended in 100 μL/well of 4° C. cold DPBS buffer containing 1:5000 diluted Fixable Viability Dye eFluor 450 (eBioscience, Cat. No. 65 0863 18). Plates were incubated for 30 minutes at 4° C. and washed once with 200 μL, 4° C. cold DPBS buffer. Afterwards cells were resuspended in 50 μL/well of 4° C. cold FACS buffer containing different titrated concentrations (starting concentration 300 nM, in 1:6 dilution in eight dilution steps) of bispecific, bivalent 2+2 anti-FAP, anti-4-1BB lipocalin huIgG1 PGLALA antigen binding molecule (termed 2+2) or bispecific, monovalent 1+2 anti-FAP, anti-4-1BB lipocalin huIgG1 PGLALA antigen binding molecule (termed 1+2) or control molecules followed by an incubation for 1 hour at 4° C. in the dark. After washing four times with with 200 μL DPBS/well, cells were stained with 50 μL/well of 4° C. cold FACS buffer containing 2.5 μg/mL PE-conjugated AffiniPure anti-human IgG Fc-fragment-specific goat F(ab′)2 fragment (Jackson ImmunoResearch, Cat.-No. 109-116-098) for 30 minutes at 4° C. Cells were washed twice with 200 μL 4° C. DPBS buffer and then resuspended in 50 μL/well DPBS containing 1% Formaldehyde for fixation. The same or the next day cells were resuspended in 100 μL FACS-buffer and acquired using MACSQuant Analyzer 10 (Miltenyi Biotec) or Canto II (BD). Data was analyzed using FlowJo 10.4.2 (FlowJo LLC), Microsoft Office Excel Professional 2010 (Microsoft Software Inc.) and GraphPad Prism (GraphPad Software Inc.).

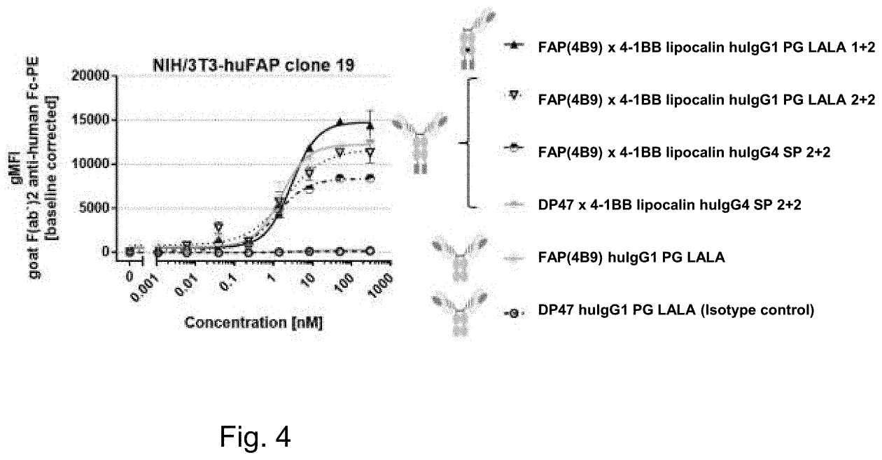

As shown in FIG. 4, the bispecific, bivalent 2+2 anti-FAP, anti-4-1BB huIgG1 PGLALA (termed FAP (4B9)×4-1BB lipocalin huIgG1 PG LALA 2+2) binds with similar affinity and as the FAP (4B9) huIgG1 PG LALA, as both molecules bind bivalent to FAP. Therefore C-terminal fusion of 4-1BB-binding lipocalins does not influence the binding to FAP. The bispecific, bivalent 2+2 anti-FAP, anti-4-1BB huIgG4 SP molecule (FAP (4B9)×4-1BB lipocalin huIgG4 SP 2+2) shows a lower gMFI than the other FAP-bivalent binding molecules. This can be explained by the different isotype of the Fc-fragment. As we are using a polyclonal anti-human Fc-fragment specific goat IgG F(ab′)2 fragment, epitopes in Fc-part may differ leading to less bound 2nd detection fragment and lower gMFI. The bispecific, monovalent 1+2 anti-FAP, anti-4-1BB huIgG1 PG LALA molecule (FAP (4B9)×4-1BB lipocalin huIgG1 PG LALA 1+2, filled black triangle and line) shows a higher gMFI than bispecific, bivalent 2+2 anti-FAP, anti-4-1BB huIgG1 PGLALA (termed FAP (4B9)×4-1BB lipocalin huIgG1 PG LALA 2+2). This can be explained by its monovalent binding to FAP, resulting in a higher occupancy on the cell surface as one molecule is only occupying one FAP-monomer instead of two. As FAP(4B9) displays a very high affinity, the loss of avidity (e.g. increase of EC₅₀ value) cannot be detected in this binding assay. EC₅₀ values and area under the curve (AUC) of the individual binding curves are listed in Table 7 and Table 8, respectively.

TABLE 7
EC₅₀ values of binding curves to FAP expressing cell line
NIH/3T3-huFAP clone 19 as shown in FIG. 4

| | FAP (4B9) x | FAP (4B9) x | FAP (4B9) | |
|---|---|---|---|---|
| | 4-1BB | 4-1BB | anti-4-1BB | |
| | lipocalin | lipocalin | lipocalin | FAP (4B9) |
| | huIgG1 PG | huIgG1 PG | huIgG4 SP | huIgG1 PG |
| EC₅₀ [nM] | LALA 1 + 2 | LALA 2 + 2 | 2 + 2 | LALA |
| NIH/3T3-huFAP | 2.88 | 2.01 | 1.19 | 1.59 |
| clone 19 | | | | |

TABLE 8
Area under the curve (AUC) values of binding curves to FAP expressing cell
line NIH/3T3-huFAP clone 19 as shown in FIG. 4

| | | | FAP (4B9) | | | |
|---|---|---|---|---|---|---|
| | FAP (4B9) | FAP (4B9) | x anti-4- | DP47 x | | |
| | x 4-1BB | x 4-1BB | 1BB | 4-1BB | | |
| | lipocalin | lipocalin | lipocalin | lipocalin | FAP (4B9) | DP47 |
| | huIgG1 PG | huIgG1 PG | huIgG4 SP | huIgG4 | huIgG1 | huIgG1 |
| AUC | LALA 1 + 2 | LALA 2 + 2 | 2 + 2 | SP 2 + 2 | PG LALA | PG LALA |
| NIH/3T3-huFAP | 32453 | 28791 | 21033 | 579 | 30424 | 362 |
| clone 19 | | | | | | |

### 3.2 Binding to Human 4-1BB Expressing Reporter Cell Line Jurkat-hu4-1BB-NFκB-luc2

For binding to cell-surface-expressed human 4-1BB (CD137) Jurkat-hu4-1BB-NFκB-luc2 reporter cell line (Promega, Germany) was used. Cells were maintained as suspension cells in RPMI 1640 medium (GIBCO by Life Technologies, Cat No 42401-042) supplied with 10% (v/v) fetal bovine serum (FBS, GIBCO by Life Technologies, Cat.-No. 16000-044, Lot 941273, gamma irradiated mycoplasma free, heat inactivated), 2 mM L-alanyl-L-glutamine dipeptide (Glutqa-MAX-I, GIBCO by Life Technologies, Cat.-No. 35050-038), 1 mM Sodium Pyruvate (SIGMA-Aldrich Cat.-No. S8636), 1% (v/v) MEM-Non essential Aminoacid Solution 100× (SIGMA-Aldrich, Cat.-No. M7145), 600 μg/ml G-418 (Roche, Cat.-No. 04727894001), 400 μg/ml Hygromycin B (Roche, Cat.-No.: 10843555001) and 25 mM HEPES (Sigma Life Science, Cat.-No.: H0887-100 mL). For the binding assay 2×10⁵ of Jurkat-hu4-1BB-NFkB-luc2 were added to each well of a round-bottom suspension cell 96-well plates (Greiner bio-one, cellstar, Cat.-No. 650185). Cells were washed once with 200 μL DPBS and pellets were resuspended in 100 μL/well of 4° C. cold DPBS buffer containing 1:5000 diluted Fixable Viability Dye eFluor 450 (eBioscience, Cat. No. 65 0863 18). Plates were incubated for 30 minutes at 4° C. and washed once with 200 μL 4° C. cold DPBS buffer. Afterwards cells were resuspended in 50 μL/well of 4° C. cold FACS buffer containing different titrated concentrations (starting concentration 300 nM, in 1:6 dilution in eight dilution steps) of bispecific, bivalent 2+2 anti-FAP, anti-4-1BB huIgG1 PGLALA (termed 2+2) or bispecific, monovalent 1+2 anti-FAP, anti-4-1BB huIgG1 PGLALA (termed 1+2) or control molecules followed by an incubation for 1 hour at 4° C. in the dark. After washing four times with with 200 μL DPBS/well, cells were stained with 50 μL/well of 4° C. cold FACS buffer containing 2.5 μg/mL PE-conjugated AffiniPure anti-human IgG Fc-fragment-specific goat F(ab′)2 fragment (Jackson ImmunoResearch, Cat.-No. 109-116-098) for 30 minutes at 4° C. Cells were washed twice with 200 μL 4° C. FACS buffer and then resuspended in 50 μL/well DPBS containing 1% Formaldehyde for fixation. The same or the next day cells were resuspended in 100 μL FACS-buffer and acquired using MACSQuant Analyzer 10 (Miltenyi Biotec) or Cantoll (BD). Data was analyzed using FlowJo 10.4.2 (FlowJo LLC), Microsoft Office Excel Professional 2010 (Microsoft Software Inc.) and GraphPad Prism (GraphPad Software Inc.).

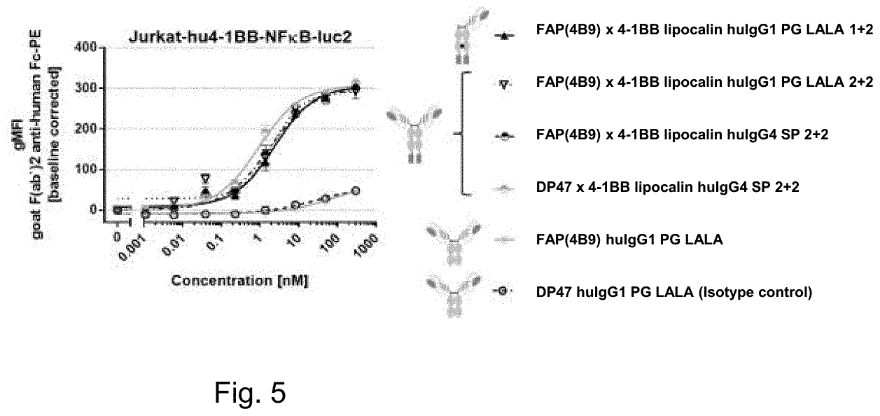

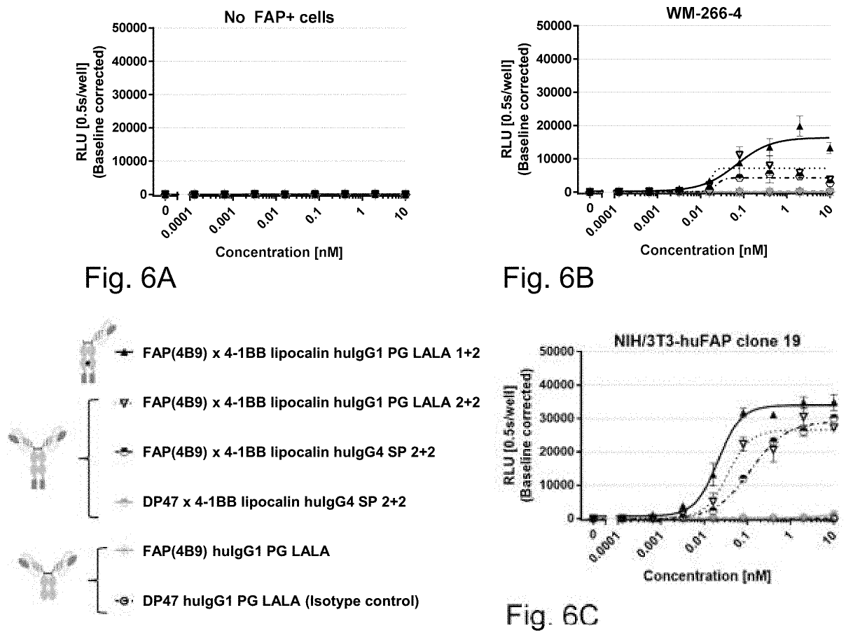

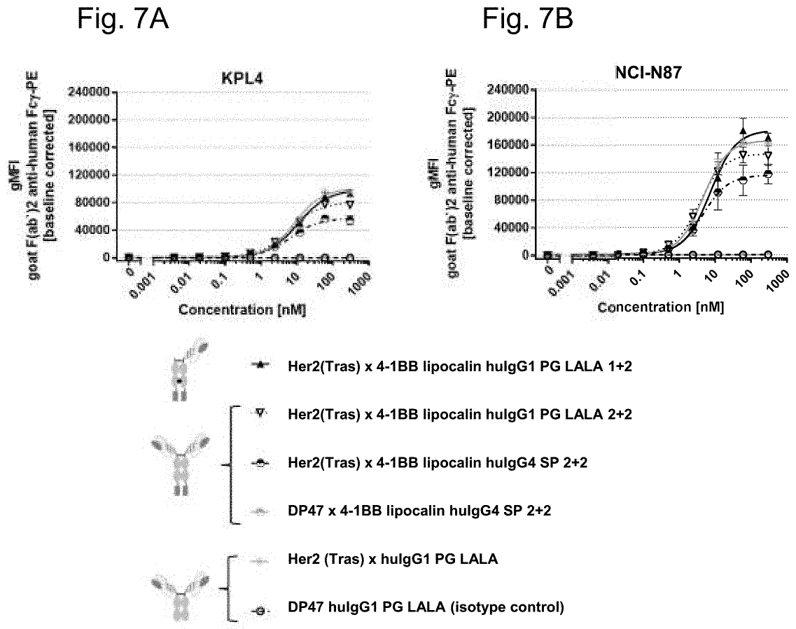

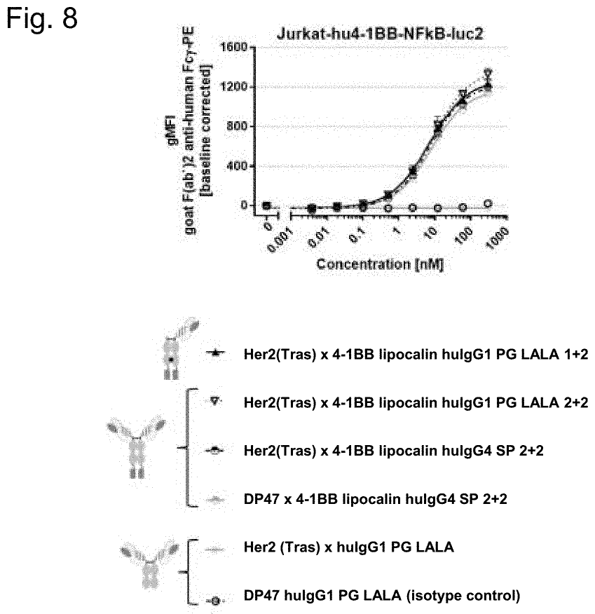

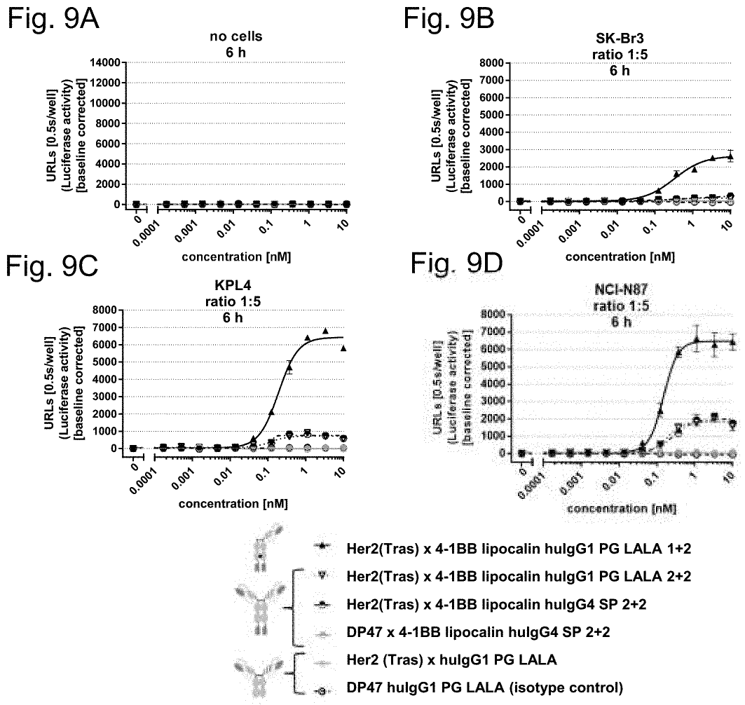

As shown in FIG. 5, all anti-4-1BB lipocalin bispecific molecules bind with a similar affinity to human 4-1BB expression transgenic human T cell lymphoma cell line Jurkat-hu4-1BB-NFkB-luc2. Different to binding to FAP expressing cells (FIG. 4) during binding to human 4-1BB we did not see a difference in binding (gMFI) between molecules containing an Fc-huIgG1 PG LALA or a Fc-huIgG4 SP. This can be related to lower expression level of 4-1BB compared to FAP and therefore much lower gMFI values, e.g. this assay is not sensitive enough to detect differences. EC₅₀ values and AUC of the binding curves are listed in Table 9 and Table 10, respectively.

TABLE 9
Summary of EC₅₀ values of binding curves to cell-expressed
human 4-1BB as shown in FIG. 5

| | FAP (4B9) x | FAP (4B9) x | FAP (4B9) | DP47 x |
|---|---|---|---|---|
| | 4-1BB | 4-1BB | anti-4-1BB | 4-1BB |
| | lipocalin | lipocalin | lipocalin | lipocalin |
| | huIgG1 PG | huIgG1 PG | huIgG4 SP | huIgG4 SP |
| EC₅₀ [nM] | LALA 1 + 2 | LALA 2 + 2 | 2 + 2 | 2 + 2 |
| Jurkat-hu4-1BB- | 2.39 | 2.09 | 1.72 | 0.92 |
| NFkB-luc2 | | | | |

TABLE 10
Summary of Area under the curve (AUC) values of binding curves to ell-
expressed human 4-1BB as shown in FIG. 5

| | | | FAP (4B9) | | | |
|---|---|---|---|---|---|---|
| | FAP (4B9) | FAP (4B9) | x anti-4- | DP47 x | | |
| | x 4-1BB | x 4-1BB | 1BB | 4-1BB | | |
| | lipocalin | lipocalin | lipocalin | lipocalin | FAP (4B9) | DP47 |
| | huIgG1 PG | huIgG1 PG | huIgG4 SP | huIgG4 | huIgG1 | huIgG1 |
| AUC | LALA 1 + 2 | LALA 2 + 2 | 2 + 2 | SP 2 + 2 | PG LALA | PG LALA |
| Jurkat-hu4- | 681 | 746 | 694 | 774 | 75 | 87 |
| 1BB-NFkB-luc2 | | | | | | |

### 3.3 NF-κB Activation in Human 4-1BB and NFκB-Luciferase Reporter Gene Expressing Reporter Cell Line Jurkat-hu4-1BB-NFκB-luc2

Agonistic binding of the 4-1BB (CD137) receptor to its ligand (4-1BBL) induces 4-1BB-downstream signaling via activation of nuclear factor kappa B (NFkB) and promotes survival and activity of CD8 T cells (Lee H W, Park S J, Choi B K, Kim H H, Nam K O, Kwon B S. 4-1BB promotes the survival of CD8 (+) T lymphocytes by increasing expression of Bch x(L) and Bfl-1. J Immunol 2002; 169:4882-4888). To monitor this NFκB-activation mediated by the bispecific, bivalent 2+2 anti-FAP, anti-4-1BB huIgG1 PGLALA molecule (termed 2+2) or the bispecific, monovalent 1+2 anti-FAP, anti-4-1BB huIgG1 PGLALA molecule (termed 1+2), Jurkat-hu4-1BB-NFκB-luc2 reporter cell line was purchased from Promega (Germany). The cells were cultured as described above (Binding to human 4-1BB expressing reporter cell line Jurkat-hu4-1BB-NFkB-luc2). For the assay cells were harvested and resuspended in assay medium RPMI 1640 medium supplied with 10% (v/v) FBS and 1% (v/v) GlutaMAX-I. 10 μl containing 2×10³ Jurkat-hu4-1BB-NFκB-luc2 reporter cells were transferred to each well of a sterile white 384-well flat bottom tissue culture plate with lid (Corning, Cat.-No.: 3826). 10 μL of assay medium containing titrated concentrations of bispecific, bivalent 2+2 anti-FAP, anti-4-1BB huIgG1 PGLALA (termed 2+2) or bispecific, monovalent 1+2 anti-FAP, anti-4-1BB huIgG1 PGLALA (termed 1+2) or control molecules were added. Finally 10 μL of assay medium alone or containing 1×10⁴ cells FAP-expressing cells, human melanoma cell line WM-266-4 (ATCC CRL-1676) or NIH/3T3-huFAP clone 19 (as described above) were supplied and plates were incubated for 6 hours at 37° C. and 5% CO₂ in a cell incubator. 6 μl freshly thawed One-Glo Luciferase assay detection solution (Promega, Cat.-No.: E6110) were added to each well and Luminescence light emission were measured immediately using Tecan microplate reader (500 ms integration time, no filter collecting all wavelength). Data was analyzed using Microsoft Office Excel Professional 2010 (Microsoft Software Inc.) and GraphPad Prism (GraphPad Software Inc.).

As shown in FIG. 6A, in the absence of FAP expressing cells, none of the molecules was able to induce strong human 4-1BB receptor activation in the Jurkat-hu4-1BB-NFκB-luc2 reporter cell line, leading to NFκB-activation and therefore Luciferase expression. In the presence of FAP-expressing cells like WM-266-4 (FIG. 6B, human melanoma cell line, intermediate FAP-expression) or NIH/3T3-huFAP clone 19 (FIG. 6C, human-FAP-transgenic mouse fibroblast cell line) crosslinking of the bispecific, bivalent 2+2 anti-FAP, anti-4-1BB huIgG1 PGLALA antigen binding molecule (termed FAP (4B9)×4-1BB lipocalin huIgG1 PG LALA 2+2, open, facing-down black triangle and dotted line) or the bispecific, monovalent 1+2 anti-FAP, anti-4-1BB huIgG1 PGLALA antigen binding molecule (termed FAP (4B9)×4-1BB lipocalin huIgG1 PG LALA 1+2, filled black triangle and line) or the bispecific control molecule bispecific, bivalent 2+2 anti-FAP, anti-4-1BB huIgG4 PGLALA antigen binding molecule (termed FAP (4B9)×4-1BB lipocalin huIgG4 SP 2+2, half-filled black hexamer and line-dotted line) let to a strong increase of NFκB-activated Luciferase activity in the Jurkat-hu4-1BB-NFκB-luc2 reporter cell line. The bispecific, monovalent 1+2 anti-FAP, anti-4-1BB huIgG1 PGLALA antigen binding molecule (termed FAP (4B9)×4-1BB lipocalin huIgG1 PG LALA 1+2, filled black triangle and line), performed the best with the highest area under the curve (AUC) of the activation curve. The lower ratio of 1:2 of tumor-target-binding side to effector-cell-target-binding, e.g. the 1:2 ratio of FAP-binding moiety to 4-1BB-binding moiety, seems to lead to a higher density of occupancy, therefore to a dense crosslinking of 4-1BB agonist on the effector cells and finally to a stronger 4-1BB receptor downstream signaling. EC₅₀ values and area under the curve (AUC) of activation curves are listed in Table 11 and Table 12, respectively.

TABLE 11
EC₅₀ values of activation curves shown in FIGS. 6B and 6C

| | FAP (4B9) x | FAP (4B9) x | FAP (4B9) |
|---|---|---|---|
| | 4-1BB | 4-1BB | anti-4-1BB |
| | lipocalin | lipocalin | lipocalin |
| | huIgG1 PG | huIgG1 PG | huIgG4 SP |
| EC₅₀ [nM] | LALA 1 + 2 | LALA 2 + 2 | 2 + 2 |
| WM-266-4 | 0.07 | 0.02 | 0.02 |
| NIH/3T3-huFAP | 0.02 | 0.04 | 0.12 |
| clone 19 | | | |

TABLE 12
Summary of Area under the curve (AUC) values of activation curves as shown
in FIGS. 6B and 6C

| | | | FAP (4B9) | | | |
|---|---|---|---|---|---|---|
| | FAP (4B9) | FAP (4B9) | x anti-4- | DP47 x | | |
| | x 4-1BB | x 4-1BB | 1BB | 4-1BB | | |
| | lipocalin | lipocalin | lipocalin | lipocalin | FAP (4B9) | DP47 |
| | huIgG1 PG | huIgG1 PG | huIgG4 SP | huIgG4 | huIgG1 | huIgG1 |
| AUC | LALA 1 + 2 | LALA 2 + 2 | 2 + 2 | SP 2 + 2 | PG LALA | PG LALA |
| WM-266-4 | 37405 | 20421 | 11663 | 355 | 384 | 191 |
| NIH/3T3-huFAP | 93198 | 65493 | 55206 | 1373 | 521 | 195 |
| clone 19 | | | | | | |

### Example 4

### Functional Characterization of the HER2-Targeted 4-1BB Lipocalin Antigen Binding Molecules

### 4.1 Binding to Human HER2-Expressing Cell Lines

For binding to cell-surface-expressed HER2 human gastric cancer line NCI-N87 (ATCC CRL-5822) and human breast adenocarcinoma cell lines KPL4 (Kawasaki Medical School) were used. NCI-N87 cells were cultured as adherent cells in RPMI 1640 medium (GIBCO by Life Technologies, Cat.-No. 42401-042) supplied with 10% (v/v) FBS (GIBCO by Life Technologies, Cat.-No. 16000-044, Lot 941273, gamma irradiated mycoplasma free, heat inactivated 35 min 56° C.) and 2 mM L-alanyl-L-glutamine (GlutaMAX-I, GIBCO, Invitrogen, Cat.-No. 35050-038). KPL4 cells were cultured as adherent cells in DMEM medium (GIBCO by life technologies, Cat.-No. 42430082) supplied with 10% (v/v) FBS and 2 mM L-alanyl-L-glutamine. For the binding assay 2×10⁵ of NCI-N87 and KPL4 were added to each well of a round-bottom suspension cell 96-well plates (Greiner bio-one, cellstar, Cat.-No. 650185). Cells were washed once with 200 μL DPBS and pellets were resuspended in 100 μL/well of 4° C. cold DPBS buffer containing 1:5000 diluted Fixable Viability Dye eFluor 450 (eBioscience, Cat. No. 65 0863 18). Plates were incubated for 30 minutes at 4° C. and washed once with 200 μL 4° C. cold DPBS buffer. Afterwards cells were resuspended in 50 μL/well of 4° C. cold FACS buffer containing different titrated concentrations (starting concentration 300 nM, in 1:6 dilution in eight dilution steps) of the bispecific, bivalent 2+2 anti-HER2, anti-4-1BB huIgG1 PGLALA antigen binding molecule (termed 2+2) or the bispecific, monovalent 1+2 anti-HER2, anti-4-1BB huIgG1 PGLALA antigen binding molecule (termed 1+2) or control molecules followed by an incubation for 1 hour at 4° C. in the dark. After washing four times with with 200 μL DPBS/well, cells were stained with 50 μL/well of 4° C. cold FACS buffer containing 2.5 μg/mL PE-conjugated AffiniPure anti-human IgG Fc-fragment-specific goat F(ab′)2 fragment (Jackson ImmunoResearch, Cat.-No. 109-116-098) for 30 minutes at 4° C. Cells were washed twice with 200 μL 4° C. DPBS buffer and then resuspended in 50 μL/well DPBS containing 1% Formaldehyde for fixation. The same or the next day cells were resuspended in 100 μL FACS-buffer and acquired using MACSQuant Analyzer 10 (Miltenyi Biotec) or Cantoll (BD). Data was analyzed using FlowJo 10.4.2 (FlowJo LLC), Microsoft Office Excel Professional 2010 (Microsoft Software Inc.) and GraphPad Prism (GraphPad Software Inc.).

As shown in FIGS. 7A and 7B, the bispecific, bivalent 2+2 anti-HER2, anti-4-1BB huIgG1 PGLALA antigen binding molecule (termed HER2 (TRAS)×4-1BB lipocalin huIgG1 PG LALA 2+2) binds with similar affinity and as the HER2 (TRAS) huIgG1 PG LALA, as both molecules bind bivalent to HER2. Therefore, C-terminal fusion of 4-1BB-binding lipocalins does not influence the binding to HER2. The bispecific, bivalent 2+2 anti-HER2, anti-4-1BB huIgG4 SP molecule (HER2 (TRAS)×4-1BB lipocalin huIgG4 SP 2+2) shows a lower MFI than the other HER2-bivalent binding molecules. This can be explained by the different Isotype of the Fc-fragment. As we are using a polyclonal anti-human Fc-fragment specific goat IgG F(ab′)2 fragment, epitopes in Fc-part may differ leading to less bound 2nd detection fragment and lower gMFI. The bispecific, monovalent 1+2 anti-HER2, anti-4-1BB huIgG1 PG LALA molecule (HER2 (TRAS)×4-1BB lipocalin huIgG1 PG LALA 1+2, filled black triangle and line) shows a higher gMFI than the bispecific, bivalent 2+2 anti-HER2, anti-4-1BB huIgG1 PGLALA antigen binding molecule (termed HER2 (TRAS)×4-1BB lipocalin huIgG1 PG LALA 2+2). This can be explained by its monovalent binding to HER2, resulting in a higher occupancy on the cell surface as one molecule is only occupying one HER2 instead of two. EC₅₀ values and area under the curve (AUC) of the individual binding curves are listed in Table 13 and Table 14, respectively.

TABLE 13
EC₅₀ values of binding curves to HER2 expressing cell lines
NCI-N87 and KPL4 as shown in FIGS. 7A and 7B

| | | HER2 | HER2 | |
|---|---|---|---|---|
| | HER2 | (TRAS) x 4- | (TRAS) anti- | |
| | (TRAS) x 4- | 1BB | 4-1BB | HER2 |
| | 1BB lipocalin | lipocalin | lipocalin | (TRAS) |
| | huIgG1 PG | huIgG1 PG | huIgG4 SP | huIgG1 PG |
| EC₅₀ [nM] | LALA 1 + 2 | LALA 2 + 2 | 2 + 2 | LALA |
| KPL4 | 10.77 | 6.19 | 6.47 | 8.69 |
| NCI-N87 | 7.40 | 3.55 | 4.54 | 4.65 |

TABLE 14
Area under the curve (AUC) values of binding curves to HER2 expressing
expressing cell lines NCI-N87 and KPL4 as shown in FIGS. 7A and 7B

| | HER2 | HER2 | HER2 | | | |
|---|---|---|---|---|---|---|
| | (TRAS) x | (TRAS) x | (TRAS) x | DP47 x | | |
| | 4-1BB | 4-1BB | 4-1BB | 4-1BB | HER2 | DP47 |
| | lipocalin | lipocalin | lipocalin | lipocalin | (TRAS) | huIgG1 |
| | huIgG1 PG | huIgG1 PG | huIgG4 SP | huIgG4 | huIgG1 | P329G |
| AUC | LALA 1 + 2 | LALA 2 + 2 | 2 + 2 | SP 2 + 2 | PG LALA | LALA |
| KPL4 | 147352 | 138815 | 98444 | 212 | 160172 | 177 |
| NCI-N87 | 300986 | 287738 | 212102 | 1426 | 306431 | 1134 |

### 4.2 Binding to Human 4-1BB Expressing Reporter Cell Line Jurkat-Hu4-1BB-NFκB-Luc2

For binding to cell-surface-expressed human 4-1BB (CD137) Jurkat-hu4-1BB-NFkB-luc2 reporter cell line (Promega, Germany) was used. Cells were maintained as suspension cells in RPMI 1640 medium (GIBCO by Life Technologies, Cat No 42401-042) supplied with 10% (v/v) fetal bovine serum (FBS, GIBCO by Life Technologies, Cat.-No. 16000-044, Lot 941273, gamma irradiated mycoplasma free, heat inactivated), 2 mM L-alanyl-L-glutamine dipeptide (GlutaMAX-I, GIBCO by Life Technologies, Cat.-No. 35050-038), 1 mM Sodium Pyruvate (SIGMA-Aldrich Cat.-No. S8636), 1% (v/v) MEM-Non essential Aminoacid Solution 100× (SIGMA-Aldrich, Cat.-No. M7145), 600 μg/ml G-418 (Roche, Cat.-No. 04727894001), 400 μg/ml Hygromycin B (Roche, Cat.-No.: 10843555001) and 25 mM HEPES (Sigma Life Science, Cat.-No.: H0887-100 mL). For the binding assay 2×10⁵ of Jurkat-hu4-1BB-NFkB-luc2 were added to each well of a round-bottom suspension cell 96-well plates (Greiner bio-one, cellstar, Cat.-No. 650185). Cells were washed once with 200 μL DPBS and pellets were resuspended in 100 μL/well of 4° C. cold DPBS buffer containing 1:5000 diluted Fixable Viability Dye eFluor 450 (eBioscience, Cat. No. 65 0863 18). Plates were incubated for 30 minutes at 4° C. and washed once with 200 μL 4° C. cold DPBS buffer. Afterwards cells were resuspended in 50 μL/well of 4° C. cold FACS buffer containing different titrated concentrations (starting concentration 300 nM, in 1:6 dilution in eight dilution steps) of the bispecific, bivalent 2+2 anti-HER2, anti-4-1BB huIgG1 PGLALA antigen binding molecule (termed 2+2) or the bispecific, monovalent 1+2 anti-HER2, anti-4-1BB huIgG1 PGLALA antigen binding molecule (termed 1+2) or control molecules followed by an incubation for 1 hour at 4° C. in the dark. After washing four times with with 200 μL DPBS/well, cells were stained with 50 μL/well of 4° C. cold FACS buffer containing 2.5 μg/mL PE-conjugated AffiniPure anti-human IgG Fc-fragment-specific goat F(ab′)2 fragment (Jackson ImmunoResearch, Cat.-No. 109-116-098) for 30 minutes at 4° C. Cells were washed twice with 200 μL 4° C. FACS buffer and then resuspended in 50 DPBS containing 1% Formaldehyde for fixation. The same or the next day cells were resuspended in 1004 FACS-buffer and acquired using MACSQuant Analyzer 10 (Miltenyi Biotec) or Cantoll (BD). Data was analyzed using FlowJo 10.4.2 (FlowJo LLC), Microsoft Office Excel Professional 2010 (Microsoft Software Inc.) and GraphPad Prism (GraphPad Software Inc.).

As shown in FIG. 8, all anti-4-1BB lipocalin bispecific molecules bind with a similar affinity to human 4-1BB expression transgenic human T cell lymphoma cell line Jurkat-hu4-1BB-NFkB-luc2. Different to binding to HER2 expressing cells (FIGS. 7A and 7B) during binding to human 4-1BB we did not see a difference in binding (gMFI) between molecules containing a Fc-huIgG1 PG LALA or a Fc-huIgG4 SP. This may be related to a lower expression level of 4-1BB compared to HER2 and therefore much lower gMFI values, e.g. this assay is not sensitive enough to detect differences. EC₅₀ values and AUC of the binding curves are listed in Table 15 and Table 16, respectively.

TABLE 15
Summary of EC₅₀ values of binding curves to cell-expressed
human 4-1BB as shown in FIG. 8

| | HER2 | HER2 | HER2 | |
|---|---|---|---|---|
| | (TRAS) x 4- | (TRAS) x 4- | (TRAS) anti- | |
| | 1BB | 1BB | 4-1BB | HER2 |
| | lipocalin | lipocalin | lipocalin | (TRAS) |
| | huIgG1 PG | huIgG1 PG | huIgG4 SP | huIgG1 PG |
| EC₅₀ [nM] | LALA 1 + 2 | LALA 2 + 2 | 2 + 2 | LALA |
| Jurkat-hu4-1BB- | 6.95 | 8.23 | 7.38 | 8.00 |
| NFkB-luc2 | | | | |

TABLE 16
Area under the curve (AUC) values of binding curves to HER2 expressing
expressing cell lines NCI-N87 and KPL4 as shown in FIG. 8

| | HER2 | HER2 | HER2 | | | |
|---|---|---|---|---|---|---|
| | (TRAS) x | (TRAS) x | (TRAS) x | DP47 x | | |
| | 4-1BB | 4-1BB | 4-1BB | 4-1BB | HER2 | DP47 |
| | lipocalin | lipocalin | lipocalin | lipocalin | (TRAS) | huIgG1 |
| | huIgG1 PG | huIgG1 PG | huIgG4 SP | huIgG4 | huIgG1 | P329G |
| AUC | LALA 1 + 2 | LALA 2 + 2 | 2 + 2 | SP 2 + 2 | PG LALA | LALA |
| Jurkat-hu4- | 2096 | 2182 | 2005 | 1876 | 143 | 101 |
| 1BB-NFkB-luc2 | | | | | | |

### 4.3 NF-κB Activation in Human 4-1BB and NFκB-Luciferase Reporter Gene Expressing Reporter Cell Line Jurkat-hu4-1BB-NFκB-luc2

Agonistic binding of the 4-1BB (CD137) receptor to its ligand (4-1BBL) induces 4-1BB-downstream signaling via activation of nuclear factor kappa B (NFkB) and promotes survival and activity of CD8 T cells (Lee H W, Park S J, Choi B K, Kim H H, Nam K O, Kwon B S. 4-1BB promotes the survival of CD8 (+) T lymphocytes by increasing expression of Bch x(L) and Bfl-1. J Immunol 2002; 169:4882-4888). To monitor this NFκB-activation mediated by the bispecific, bivalent 2+2 anti-HER2, anti-4-1BB huIgG1 PGLALA antigen binding molecule (termed 2+2) or the bispecific, monovalent 1+2 anti-HER2, anti-4-1BB huIgG1 PGLALA antigen binding molecule (termed 1+2), Jurkat-hu4-1BB-NFκB-luc2 reporter cell line was purchased from Promega (Germany). The cells were cultured as described above (Binding to human 4-1BB expressing reporter cell line Jurkat-hu4-1BB-NFkB-luc2). For the assay, cells were harvested and resuspended in assay medium RPMI 1640 medium supplied with 10% (v/v) FBS and 1% (v/v) GlutaMAX-I. 10 μl containing 2×10³ Jurkat-hu4-1BB-NFκB-luc2 reporter cells were transferred to each well of a sterile white 384-well flat bottom tissue culture plate with lid (Corning, Cat.-No.: 3826). 10 μL of assay medium containing titrated concentrations of the bispecific, bivalent 2+2 anti-HER2, anti-4-1BB huIgG1 PGLALA antigen binding molecule (termed 2+2) or the bispecific, monovalent 1+2 anti-HER2, anti-4-1BB huIgG1 PGLALA antigen binding molecule (termed 1+2) or control molecules were added. Finally, 10 μL of assay medium alone or containing 1×10⁴ cells HER2-expressing cells KPL4, NCI-N87 (as described above) or SK-Br3 (Human breast adenocarcinoma, ATCC HTB-30) were supplied and plates were incubated for 6 hours at 37° C. and 5% CO₂ in a cell incubator. 6 μl freshly thawed One-Glo Luciferase assay detection solution (Promega, Cat.-No.: E6110) were added to each well and Luminescence light emission were measured immediately using Tecan microplate reader (500 ms integration time, no filter collecting all wavelength).

As shown in the FIGS. 9A to 9D, in the absence of HER2 expressing cells (FIG. 9A), none of the molecules was able to induce strong human 4-1BB receptor activation in the Jurkat-hu4-1BB-NFkB-luc2 reporter cell line, leading to NFkB-activation and therefore Luciferase expression. In the presence of HER2-expressing cells like SK-Br3 (FIG. 9B), KPL4 (FIG. 9C) and NCI-N87 (FIG. 9D) crosslinking of the bispecific, monovalent 2+1 anti-HER2, anti-4-1BB huIgG1 PGLALA antigen binding molecule (termed HER2 (TRAS)×4-1BB lipocalin huIgG1 PG LALA 2+1, filled black triangle and line) shows good activation curves correlating in their height and/or EC₅₀ values with the strength of HER2 expression of crosslinking cells. The bispecific, bivalent 2+2 anti-HER2, anti-4-1BB huIgG1 PGLALA antigen binding molecule (termed HER2 (TRAS)×4-1BB lipocalin huIgG1 PG LALA 2+2, filled black triangle and line) and its control molecule HER2 (TRAS)×4-1BB lipocalin huIgG4 SP (half-filled black hexamer and dotted line) bind both bivalent to HER2 and induce similar activation curves, whereby the activation of both molecules are far below the activation curves of HER2 (TRAS)×4-1BB lipocalin huIgG1 PG LALA 2+1 (black filled triangle and line). The bispecific, monovalent 1+2 anti-HER2, anti-4-1BB huIgG1 PGLALA (termed HER2 (TRAS)×4-1BB lipocalin huIgG1 PG LALA 1+2, filled black triangle and line), performed the best with the highest area under the curve (AUC) of the activation curve. We believe, that the lower ratio of 1:2 of tumor-target-binding side to effector-cell-target-binding, e.g. the 1:2 ratio of HER2-binding moiety to 4-1BB-binding moiety, leads to a higher density of occupancy on the tumor cells, therefore a dense crosslinking of 4-1BB agonist on the effector cells and finally to a stronger 4-1BB receptor downstream signaling. EC₅₀ values and area under the curve (AUC) of activation curves are listed in Table 17 and Table 18, respectively.

TABLE 17
Summary of EC₅₀ values of activation curves as
shown in FIGS. 9B, 9C and 9D

| | | HER2 | HER2 |
|---|---|---|---|
| | HER2 (TRAS) | (TRAS) x 4- | (TRAS) |
| | x 4-1BB | 1BB | anti-4-1BB |
| | lipocalin | lipocalin | lipocalin |
| | huIgG1 PG | huIgG1 PG | huIgG4 SP |
| EC₅₀ [nM] | LALA 1 + 2 | LALA 2 + 2 | 2 + 2 |
| SK-Br3 | 0.33 | 0.19 | 1.55 |
| KPL4 | 0.19 | 0.12 | 0.13 |
| NCI-N87 | 0.15 | 0.19 | 0.27 |

TABLE 18
Area under the curve (AUC) values of activation curves as shown in FIGS.
9B, 9C and 9D

| | HER2 | HER2 | HER2 | | | |
|---|---|---|---|---|---|---|
| | (TRAS) x | (TRAS) x | (TRAS) x | DP47 x | | |
| | 4-1BB | 4-1BB | 4-1BB | 4-1BB | | HER2 |
| | lipocalin | lipocalin | lipocalin | lipocalin | DP47 | (TRAS) |
| | huIgG1 PG | huIgG1 PG | huIgG4 SP | huIgG4 | huIgG1 | huIgG1 |
| AUC | LALA 1 + 2 | LALA 2 + 2 | 2 + 2 | SP 2 + 2 | PG LALA | PG LALA |
| SK-Br3 | 3963 | 384 | 464 | 101 | 129 | 289 |
| KPL4 | 11364 | 1721 | 1554 | 78 | 253 | 95 |
| NCI-N87 | 12121 | 3456 | 3311 | 394 | 127 | 62 |

1. A bispecific antigen binding molecule capable of bivalent binding to 4-1BB and monovalent binding to a target cell antigen, comprising
(a) an antigen binding domain capable of specific binding to a target cell antigen,
(b) an Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

2. The bispecific antigen binding molecule of claim 1, wherein each of the lipocalin muteins capable of specific binding to 4-1BB is derived from mature human neutrophil gelatinase-associated lipocalin (huNGAL) of SEQ ID NO:1.

3. The bispecific antigen binding molecule of claim 1, wherein each of the lipocalin muteins capable of specific binding to 4-1BB comprise the amino acid sequence of SEQ ID NO:2 or an amino acid sequence of SEQ ID NO:2, wherein one or more of the following amino acids are mutated as follows:
(a) Q at position 20 is replaced by R,
(b) N at position 25 is replaced by Y or D,
(c) H at position 28 is replaced by Q,
(d) Q at position 36 is replaced by M,
(e) I at position 40 is replaced by N,
(f) R at position 41 is replaced by L or K, or
(g) E at position 44 is replaced by V or D,
(h) K at position 46 is replaced by S and the amino acids at positions 47 to 49 are deleted,
(i) I at position 49 is replaced by H, N, V or S,
(j) M at position 52 is replaced by S or G,
(k) K at position 59 is replaced by N,
(l) D at position 65 is replaced by N,
(m) M at position 68 is replaced by D, G or A,
(n) K at position 70 is replaced by M, T, A or S,
(o) F at position 71 is replaced by L,
(p) D at position 72 is replaced by L,
(q) M at position 77 is replaced by Q, H, T, R or N,
(s) D at position 79 is replaced by I or A,
(t) I at position 80 is replaced by N,
(u) W at position 81 is replaced by Q, S or M,
(v) T at position 82 is replaced by P,
(w) F at position 83 is replaced by L,
(y) F at position 92 is replaced by L or S,
(z) L at position 94 is replaced by F,
(za) K at position 96 is replaced by F,
(zb) F at position 100 is replaced by D,
(zc) P at position 101 is replaced by L,
(zd) H at position 103 is replaced by P,
(ze) S at position 106 is replaced by Y,
(zf) F at position 122 is replaced by Y,
(zg) F at position 125 is replaced by S,
(zh) F at position 127 it replaced by I,
(zi) E at position 132 is replaced by W, or
(zj) Y at position 134 is replaced by G.

4. The bispecific antigen binding molecule of claim 1, wherein each of the lipocalin muteins capable of specific binding to 4-1BB comprise an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 and SEQ ID NO:20.

5. The bispecific antigen binding molecule of claim 1, wherein each of the lipocalin muteins capable of specific binding to 4-1BB comprise the amino acid sequence of SEQ ID NO:2.

6. The bispecific antigen binding molecule of claim 1, wherein the Fc domain comprises knob-into-hole modifications promoting association of the first and the second subunit of the Fc domain.

7. The bispecific antigen binding molecule of claim 1, wherein the Fc domain comprises one or more amino acid substitution that reduces binding to an Fc receptor, in particular towards Fcγ receptor.

8. The bispecific antigen binding molecule of claim 1, wherein the Fc domain is an IgG1 Fc domain comprising the amino acid substitutions the amino acid substitutions L234A, L235A and P329G (EU numbering according to Kabat).

9. The bispecific antigen binding molecule of claim 1, wherein the antigen binding domain capable of specific binding to a target cell antigen is a Fab fragment capable of specific binding to a target cell antigen.

10. The bispecific antigen binding molecule of claim 9, wherein the Fab fragment capable of specific binding to a target cell antigen is a Fab fragment capable of specific binding to Fibroblast Activation Protein (FAP).

11. The bispecific antigen binding molecule of claim 10, wherein the Fab fragment capable of specific binding to Fibroblast Activation Protein (FAP) comprises
(a) a heavy chain variable region (V_{H}FAP) comprising (i) CDR-H1 comprising the amino acid sequence of SEQ ID NO:21, (ii) CDR-H2 comprising the amino acid sequence of SEQ ID NO:22, and (iii) CDR-H3 comprising the amino acid sequence of SEQ ID NO:23, and a light chain variable region (V_{L}FAP) comprising (iv) CDR-L1 comprising the amino acid sequence of SEQ ID NO:24, (v) CDR-L2 comprising the amino acid sequence of SEQ ID NO:25, and (vi) CDR-L3 comprising the amino acid sequence of SEQ ID NO:26, or
(b) a heavy chain variable region (V_{H}FAP) comprising (i) CDR-H1 comprising the amino acid sequence of SEQ ID NO:29, (ii) CDR-H2 comprising the amino acid sequence of SEQ ID NO:30, and (iii) CDR-H3 comprising the amino acid sequence of SEQ ID NO:31, and a light chain variable region (V_{L}FAP) comprising (iv) CDR-L1 comprising the amino acid sequence of SEQ ID NO:32, (v) CDR-L2 comprising the amino acid sequence of SEQ ID NO:33, and (vi) CDR-L3 comprising the amino acid sequence of SEQ ID NO:34.

12. The bispecific antigen binding molecule of claim 10, wherein the Fab fragment capable of specific binding to Fibroblast Activation Protein (FAP) comprises
(a) a heavy chain variable region (V_{H}FAP) comprising an amino acid sequence that is at least about 95%, 96%, 97%, 98%, 99% or 100% identical to the amino acid sequence of SEQ ID NO:27, and a light chain variable region (V_{L}FAP) comprising an amino acid sequence that is at least about 95%, 96%, 97%, 98%, 99% or 100% identical to the amino acid sequence of SEQ ID NO:28, or
(b) a heavy chain variable region (V_{H}FAP) comprising an amino acid sequence that is at least about 95%, 96%, 97%, 98%, 99% or 100% identical to the amino acid sequence of SEQ ID NO:35, and a light chain variable region (V_{L}FAP) comprising an amino acid sequence that is at least about 95%, 96%, 97%, 98%, 99% or 100% identical to the amino acid sequence of SEQ ID NO:36.

13. The bispecific antigen binding molecule of claim 1, comprising a first heavy chain of SEQ ID NO:37, a second heavy chain of SEQ ID NO:38 and a light chain of SEQ ID NO:39.

14. The bispecific antigen binding molecule of claim 9, wherein the Fab fragment capable of specific binding to a target cell antigen is a Fab fragment capable of specific binding to HER2.

15. The bispecific antigen binding molecule of any one of claim 14, wherein the Fab fragment capable of specific binding to HER2 comprises
(a) a VH domain comprising (i) CDR-H1 comprising the amino acid sequence of SEQ ID NO:40, (ii) CDR-H2 comprising the amino acid sequence of SEQ ID NO:41, and (iii) CDR-H3 comprising the amino acid sequence of SEQ ID NO:42, and a VL domain comprising (iv) CDR-L1 comprising the amino acid sequence of SEQ ID NO:43, (v) CDR-L2 comprising the amino acid sequence of SEQ ID NO:44, and (vi) CDR-L3 comprising the amino acid sequence of SEQ ID NO:45, or
(b) a VH domain comprising (i) CDR-H1 comprising the amino acid sequence of SEQ ID NO:48, (ii) CDR-H2 comprising the amino acid sequence of SEQ ID NO:49, and (iii) CDR-H3 comprising the amino acid sequence of SEQ ID NO:50, and a VL domain comprising (iv) CDR-L1 comprising the amino acid sequence of SEQ ID NO:51, (v) CDR-L2 comprising the amino acid sequence of SEQ ID NO:52, and (vi) CDR-L3 comprising the amino acid sequence of SEQ ID NO:53, or
(c) a VH domain comprising (i) CDR-H1 comprising the amino acid sequence of SEQ ID NO:56, (ii) CDR-H2 comprising the amino acid sequence of SEQ ID NO:57, and (iii) CDR-H3 comprising the amino acid sequence of SEQ ID NO:58, and a VL domain comprising (iv) CDR-L1 comprising the amino acid sequence of SEQ ID NO:59, (v) CDR-L2 comprising the amino acid sequence of SEQ ID NO:60, and (vi) CDR-L3 comprising the amino acid sequence of SEQ ID NO:61.

16. The bispecific antigen binding molecule of claim 14, wherein the Fab fragment capable of specific binding to HER2 comprises
(a) a heavy chain variable region (V_{H}HER2) comprising an amino acid sequence that is at least about 95%, 96%, 97%, 98%, 99% or 100% identical to the amino acid sequence of SEQ ID NO:46, and a light chain variable region (V_{L}HER2) comprising an amino acid sequence that is at least about 95%, 96%, 97%, 98%, 99% or 100% identical to the amino acid sequence of SEQ ID NO:47, or
(b) a heavy chain variable region (V_{H}HER2) comprising an amino acid sequence that is at least about 95%, 96%, 97%, 98%, 99% or 100% identical to the amino acid sequence of SEQ ID NO:54, and a light chain variable region (V_{L}HER2) comprising an amino acid sequence that is at least about 95%, 96%, 97%, 98%, 99% or 100% identical to the amino acid sequence of SEQ ID NO:55, or
(c) a heavy chain variable region (V_{H}HER2) comprising an amino acid sequence that is at least about 95%, 96%, 97%, 98%, 99% or 100% identical to the amino acid sequence of SEQ ID NO:62, and a light chain variable region (V_{L}HER2) comprising an amino acid sequence that is at least about 95%, 96%, 97%, 98%, 99% or 100% identical to the amino acid sequence of SEQ ID NO:63.

17. The bispecific antigen binding molecule of claim 14, comprising a first heavy chain of SEQ ID NO:64, a second heavy chain of SEQ ID NO:65 and a light chain of SEQ ID NO:66.

18. Isolated nucleic acid encoding the bispecific antigen binding molecule claim 1.

19. A vector comprising the isolated nucleic acid of claim 18.

20. A host cell comprising the nucleic acid of claim 18.

21. A method of producing the bispecific antigen binding molecule of claim 1, comprising culturing the host cell of claim 20 under conditions suitable for expression of the bispecific antigen binding molecule.

22. The method of claim 21, further comprising recovering the bispecific antigen binding molecule from the host cell.

23. The vector of claim 19, wherein the vector comprises an expression vector.

24. A host cell comprising the expression vector of claim 23.

25. A method of producing the bispecific antigen binding molecule of claim 1, comprising culturing the host cell of claim 24 under conditions suitable for expression of the bispecific antigen binding molecule.

26. The method of claim 25, further comprising recovering the bispecific antigen binding molecule from the host cell.

27. A pharmaceutical composition comprising the bispecific antigen binding molecule of claim 1 and at least one pharmaceutically acceptable excipient.

28. The pharmaceutical composition of claim 27, further comprising an additional therapeutic agent.

29. A method of treating an individual having cancer or an infectious disease, comprising administering to the individual an effective amount of the bispecific antigen binding molecule of claim 1, or the pharmaceutical composition comprising the bispecific antigen binding molecule.

30. A method of up-regulating or prolonging cytotoxic T cell activity in an individual having cancer, comprising administering to the individual an effective amount of the bispecific antigen binding molecule of claim 1, or the pharmaceutical composition comprising the bispecific antigen binding molecule.

31. A bispecific antigen binding molecule capable of bivalent binding to 4-1BB and monovalent binding to a target cell antigen, comprising
(a) a Fab fragment comprising an antigen binding domain capable of specific binding Fibroblast Activation Protein (FAP),
(b) an Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.

32. A bispecific antigen binding molecule capable of bivalent binding to 4-1BB and monovalent binding to a target cell antigen, comprising
(a) a Fab fragment comprising an antigen binding domain capable of specific binding to HER2,
(b) an Fc domain composed of a first and a second subunit capable of stable association, and
(c) two lipocalin muteins capable of specific binding to 4-1BB, wherein one of the lipocalin muteins is fused to the C-terminus of the first subunit of the Fc domain and the other is fused to the C-terminus of the second subunit of the Fc domain.
